SEQUENCE LISTING (26 sequences)

SEQ ID NO: 1 - protein, 200 aa, Homo sapiens
MSMLFYTLITAFLIGIQAEPHSESNVPAGHTIPQVHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSY

SEQ ID NO: 2 - DNA, 726 bp, Homo sapiens
atgtccatgttgttctacactctgatcacagcttttctgatcggcatacaggcggaaccacactcagagagcaatgtccctgcaggacacaccatcccccaagtccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 3 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 4 - DNA, 636 bp, Homo sapiens
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 5 - DNA, 615 bp, Artificial Sequence
  CAG Promoter
ataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactg

SEQ ID NO: 6 - protein, 735 aa, Dependoparvovirus adeno-associated dependoparvovirus A [sequence omitted]

SEQ ID NO: 7 - DNA, 2208 bp, Dependoparvovirus adeno-associated dependoparvovirus A
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - protein, 533 aa, Dependoparvovirus adeno-associated dependoparvovirus A
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 1602 bp, Dependoparvovirus adeno-associated dependoparvovirus A
atggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 10 - protein, 724 aa, Dependoparvovirus adeno-associated dependoparvovirus A
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - DNA, 2175 bp, Dependoparvovirus adeno-associated dependoparvovirus A
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 12 - protein, 738 aa, Dependoparvovirus adeno-associated dependoparvovirus A
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 13 - DNA, 2214 bp, Dependoparvovirus adeno-associated dependoparvovirus A
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 14 - protein, 736 aa, Dependoparvovirus adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 2209 bp, Dependoparvovirus adeno-associated dependoparvovirus A
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgt

SEQ ID NO: 16 - DNA, 613 bp, Artificial Sequence
  CMV Promoter
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaat

SEQ ID NO: 17 - DNA, 726 bp, Artificial Sequence
  Codon optimized NGF
atgagcatgctgttttacaccctgatcaccgccttcctgatcggcatccaggccgagccccacagcgagagcaacgtgcccgctggacacacaatccctcaggcccactggacaaaactgcagcatagcctggatacagccctgagaagggccagaagcgcccctgccgccgccatcgccgctagagtggccggccagacaagaaatatcaccgtggacccccggctgttcaagaaaagaagactgcggtctcctagagtgctcttttcaacacagcctcctcgggaagccgctgatacccaggacctggacttcgaggtgggaggcgccgctcctttcaatagaacccacagatccaagagaagctctagccaccctatcttccaccggggcgagttcagcgtgtgcgacagcgtttctgtgtgggtgggagataagaccaccgcaaccgatatcaagggcaaggaagtgatggtgctgggcgaagtgaacatcaacaacagcgtctttaagcagtacttcttcgagacaaagtgccgggacccaaaccccgtggacagcggctgcagaggcattgactccaagcactggaactcctactgcaccacaacacacaccttcgtgaaggccctgaccatggacggcaaacaagctgcctggcggttcatcagaatcgacaccgcctgtgtctgtgtgctgagcagaaaggccgtgcgccgggcctag

SEQ ID NO: 18 - DNA, 635 bp, Artificial Sequence
  Codon Optimized GDNF
atgaactgtgggacgttgtggccgtgtgcctggtcctcctgcacaccgcctccgcctttccactgcctgctggaaagcggcctcctgaggcccctgctgaagatagaagcctgggcagaagaagagcccccttcgccctgagctctgatagcaacatgcctgaggactaccccgaccagttcgacgacgtgatggactttatccaggccaccatcaagagactgaaacggagccctgacaagcagatggccgtgctgcctagacgggaaagaaatagacaggccgcagctgccaaccccgagaacagcagaggcaaaggccggagaggccagaggggaaaaaacagaggatgtgtgctgaccgccatccatctgaacgtgacagatctgggcctgggctatgagaccaaggaagagctgatcttcagatactgcagcggcagctgcgacgccgccgagacaacctacgacaagatcctgaagaacctgtcccggaatcggcggctggtgtctgacaaggtgggccaagcttgctgcagaccaattgccttcgacgatgatctgtctttcctggatgacaacctggtgtaccacatcctgagaaagcacagcgccaagcgctgcggctgtatctag

SEQ ID NO: 19 - DNA, 145 bp, Artificial Sequence
  5′ ITR Sequence
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaac

SEQ ID NO: 20 - DNA, 145 bp, Artificial Sequence
  3′ ITR Sequence
gttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 21 - DNA, 228 bp, Artificial Sequence
  bGHpolyA Sequence
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 22 - DNA, 411 bp, Artificial Sequence
  WPRE Sequence
tcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtc

SEQ ID NO: 23 - DNA, 2967 bp, Artificial Sequence
  Expression cassette comprising sequence encoding GDNF
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacggatctttacaaattcaagccaggtgatttcaacaaattttgctgacgatttaggcgcactatcccctaaactacaaattagaaaatagcgttccttgacactagtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaataagctttctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccgccatgaagctgtgggacgttgtggccgtgtgcctggtcctcctgcacaccgcctccgcctttccactgcctgctggaaagcggcctcctgaggcccctgctgaagatagaagcctgggcagaagaagagcccccttcgccctgagctctgatagcaacatgcctgaggactaccccgaccagttcgacgacgtgatggactttatccaggccaccatcaagagactgaaacggagccctgacaagcagatggccgtgctgcctagacgggaaagaaatagacaggccgcagctgccaaccccgagaacagcagaggcaaaggccggagaggccagaggggaaaaaacagaggatgtgtgctgaccgccatccatctgaacgtgacagatctgggcctgggctatgagaccaaggaagagctgatcttcagatactgcagcggcagctgcgacgccgccgagacaacctacgacaagatcctgaagaacctgtcccggaatcggcggctggtgtctgacaaggtgggccaagcttgctgcagaccaattgccttcgacgatgatctgtctttcctggatgacaacctggtgtaccacatcctgagaaagcacagcgccaagcgctgcggctgtatctagtcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagcgctggctagaattacctaccggcctccaccataccttcgatattcgcgcccactctcccattaatccgcacaagtggatgtgatgcgattgcccgctaagatagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 24 - DNA, 3054 bp, Artificial Sequence
  Expression cassette comprising sequence encoding NGF
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacggatctttacaaattcaagccaggtgatttcaacaaattttgctgacgatttaggcgcactatcccctaaactacaaattagaaaatagcgttccttgacactagtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaataagctttctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccgccatgagcatgctgttttacaccctgatcaccgccttcctgatcggcatccaggccgagccccacagcgagagcaacgtgcccgctggacacacaatccctcaggcccactggacaaaactgcagcatagcctggatacagccctgagaagggccagaagcgcccctgccgccgccatcgccgctagagtggccggccagacaagaaatatcaccgtggacccccggctgttcaagaaaagaagactgcggtctcctagagtgctcttttcaacacagcctcctcgggaagccgctgatacccaggacctggacttcgaggtgggaggcgccgctcctttcaatagaacccacagatccaagagaagctctagccaccctatcttccaccggggcgagttcagcgtgtgcgacagcgtttctgtgtgggtgggagataagaccaccgcaaccgatatcaagggcaaggaagtgatggtgctgggcgaagtgaacatcaacaacagcgtctttaagcagtacttcttcgagacaaagtgccgggacccaaaccccgtggacagcggctgcagaggcattgactccaagcactggaactcctactgcaccacaacacacaccttcgtgaaggccctgaccatggacggcaaacaagctgcctggcggttcatcagaatcgacaccgcctgtgtctgtgtgctgagcagaaaggccgtgcgccgggcctagtcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagcgctggctagaattacctaccggcctccaccataccttcgatattcgcgcccactctcccattaatccgcacaagtggatgtgatgcgattgcccgctaagatagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcg

SEQ ID NO: 25 - DNA, 5895 bp, Artificial Sequence
  pAAV-CMVE-NGF
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggattgacctaacgaaaagtgaacttcataatacgtgctgtcccacgcacatggtagatttggacaaaattgaatggagtctgatcaaccttcacaccgatctagatatcgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacggatctttacaaattcaagccaggtgatttcaacaaattttgctgacgatttaggcgcactatcccctaaactacaaattagaaaatagcgttccttgacactagtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaataagctttctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccgccatgagcatgctgttttacaccctgatcaccgccttcctgatcggcatccaggccgagccccacagcgagagcaacgtgcccgctggacacacaatccctcaggcccactggacaaaactgcagcatagcctggatacagccctgagaagggccagaagcgcccctgccgccgccatcgccgctagagtggccggccagacaagaaatatcaccgtggacccccggctgttcaagaaaagaagactgcggtctcctagagtgctcttttcaacacagcctcctcgggaagccgctgatacccaggacctggacttcgaggtgggaggcgccgctcctttcaatagaacccacagatccaagagaagctctagccaccctatcttccaccggggcgagttcagcgtgtgcgacagcgtttctgtgtgggtgggagataagaccaccgcaaccgatatcaagggcaaggaagtgatggtgctgggcgaagtgaacatcaacaacagcgtctttaagcagtacttcttcgagacaaagtgccgggacccaaaccccgtggacagcggctgcagaggcattgactccaagcactggaactcctactgcaccacaacacacaccttcgtgaaggccctgaccatggacggcaaacaagctgcctggcggttcatcagaatcgacaccgcctgtgtctgtgtgctgagcagaaaggccgtgcgccgggcctagtcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagcgctggctagaattacctaccggcctccaccataccttcgatattcgcgcccactctcccattaatccgcacaagtggatgtgatgcgattgcccgctaagatagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcgatatcgaatcgaatgcagtgatctcccaggtgcgaatcaaaaatactaggtaactagaggatttgcgacgttctaagcgttggtccatgtgaatcgccatccaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 26 - protein, 43 aa, Homo sapiens
SDKPDMAEIEKFDKSKLKKTETQEKNPLPSKETIEQEKQAGES

(19) United States
(12) Patent Application Publication
(10) Pub. No.: US 2024/0226109 A1
(43) Pub. Date: Jul. 11, 2024

(54) CO-THERAPY WITH VECTOR AND NICOTINIC AGONIST

(71) Applicant: OYSTER POINT PHARMA, INC., Princeton, NJ (US)

(72) Inventors: Jeffrey Alan NAU, Princeton, NJ (US); Eric C. CARLSON, Princeton, NJ (US)

(21) Appl. No.: 18/559,518

(22) PCT Filed: May 4, 2022
(86) PCT No.: PCT/US2022/027653

§ 371 Date: Nov. 7, 2023

Related U.S. Application Data
(60) Provisional application No. 63/185,897, filed on May 7, 2021.

(51) Int. Cl. A61K 31/55; A61K 9/00; A61K 31/506; A61K 38/18; A61K 48/00; A61P 27/02

(52) U.S. Cl. CPC A61K 31/55; A61K 9/0043; A61K 9/0048; A61K 31/506; A61K 38/185; A61K 48/0033; A61P 27/02

(57) ABSTRACT

Methods that increase bioavailability and/or efficacy of a therapeutic agent for treatment of an ocular surface disorder, cornea disorder, or anterior chamber disorder are provided. In particular, tear production is increased by administration of an electrical stimulus, ultrasound stimulus, and/or a drug such as an nAChRα7 agonist (e.g., varenicline, (R)-5-((E)-2-pyrrolidin-3-ylvinyl)pyrimidine, 5-{(E)-2-[(3R)-pyrrolidin-3-yl]vinyl}pyrimidine, or (R,E)-5-(2-pyrrolidine-3-yl)vinyl)pyrimidine). The primary therapeutic agents provided include recombinant adeno-associated virus (AAV) vectors expressing a therapeutic gene product (e.g., a biologic drug). Related compositions methods of use are also provided.

### CROSS-RELATED APPLICATIONS

This application claims the benefit of U.S. Provisional Patent Application Ser. No. 63/185,897 filed May 7, 2021. The entire contents of which is incorporate herein by this reference.

### REFERENCE TO SEQUENCE LISTING

The instant application contains a Sequence Listing which has been submitted electronically in ASCII format via EFS-Web, and is hereby incorporated by reference in its entirety. Said ASCII copy, created on Apr. 29, 2022, is named OYST_016_02 WO_SeqList_ST25 and is 72,423 bytes in size.

### TECHNICAL FIELD

The invention relates generally to medical treatment of ocular surface and cornea disorders, in particular to improving treatment by co-administration of an agent that increases tear production.

### BACKGROUND

Medical treatment of dry eye disease; bacterial, fungal, and viral keratitis; neurotrophic keratopathy, blepharitis, Sjogren's syndrome; allergic, vernal, viral, and bacterial conjunctivitis; glaucoma; corneal neovascularization; pterygium; corneal dystrophies (keratoconus, Fuch's dystrophy, lattice dystrophy and map-dot-fingerprint dystrophy) and other ocular surface and cornea disorders are known. In addition, medical treatment of glaucoma, presbyopia, myopia, and other anterior chamber disorders are known. These medical treatments include topical eye drops, small molecule drugs, biologic drugs, and others.

Therapeutic agents used to treat eye disorders include cyclosporine (marketed as RESTASIS, ZYCLORIN, or CYCLOKAT); lifitegrast (XIIDRA); immunomodulators (voclosporin, tacrolimus, pimecrolimus); Janus kinase (JAK) 3 inhibitors (tasocitinib); secretagogues or mucoprotective agents (diquafosol, rebamipide, ecabet); disaccharide protectants (trehalose); nonsteroidal anti-inflammatory drugs (bromfenac); steroids (dexamethasone, EGP-437, corticosteroids, prednisolone, methylprednisolone, fluorometholone, and loteprednol containing agents); steroid-sparing drugs (mycophenolate, azathioprine, cyclophosphamide); fatty acids (omega-3 fatty acids, resolvin E1); broad-spectrum antimicrobial (fluoroquinolones), anti-viral or anti-fungal agents; antibiotics (tetracycline and its derivatives (doxycycline), macrolide antibiotic (azithromycin); autologous serum; synthetic lubricants; topical vitamin A; mast cell stabilizers (e.g. nedocromil, lodoxamide), antihistamines (e.g. emedastine, loratidine, chlorphenamine), or combined mast cell stabilizers/antihistamine (e.g. olopatadine); lubricants (hyaluronate, carmellose, hypromellose, polyvinyl alcohol, paraffin, or lubricants with lipids or osmoprotectants (e.g. glycerine and L-carnitine), N-acetylcysteine); ointments containing emollients (petrolatum, mineral oil, lanolin, castor oil). In addition, anterior chamber disorders such as glaucoma are treated with prostaglandin analogs, beta blockers, alpha agonists, carbonic anhydrase inhibitors, and rho kinase inhibitors.

In principle, it is possible to delivery therapeutic agents to the eye using vectors such as viral vectors. Vaccinia, adenovirus, and adeno-associated virus (AAV) vectors have been shown to transduce primary cultures of lacrimal gland cells and/or lacrimal gland tissues, e.g, Rocha et al. IOVS 52:9567-72 (2011); Nominato et al. IOVS 59:6036-44 (2018).

Improvement to medical treatment of ocular surface disorders and anterior chamber disorders would be desirable.

### SUMMARY

The disclosure provides combination therapies and methods for improving medical treatment of ocular surface and cornea disorders by increasing tear production in the subject. Bioavailability and/or efficacy of one or more primary therapeutic agents may be improved when tear production is increased by administration of an electrical stimulus, ultrasound stimulus, and/or a drug. For example, the amount, distribution, and/or rate of delivery to the ocular surface of a primary therapeutic agent may be improved. Increased tear production is particularly effective in improving delivery to the eye of gene product(s), such as protein(s), antibodies or fragment antibodies (e.g. Fabs), or nucleic acids, recombinantly expressed in tissues adjacent to the eye.

Accordingly, the disclosure provides methods of increasing bioavailability of a primary therapeutic agent by administering a treatment that increases tear production. The treatment that increases tear production may be, without limitation, an electrical stimulus, ultrasound stimulus, a small-molecule drug, or a biologic drug. In particular embodiments, the small-molecule drug is a nicotinic acetylcholine receptor (nAChR) agonist.

Various primary therapeutic agents, and viral or non-viral vectors comprising a polynucleotide encoding a primary therapeutic agent, may be used in the methods of the present disclosure. The primary therapeutic agent may be, without limitation, a small molecule drug or biologic drug (e.g. a protein or nucleic acid).

The disclosure further provides methods of delivering a gene product to the tear film of a subject's eye by administering a viral vector (e.g., vaccinia, adenovirus, or adeno-associated virus) and nicotinic acetylcholine receptor (nAChR) agonist. The viral vector is provided in a composition suitable for injection into the subject or topical administration to the subject near the eye (e.g, the lacrimal gland). This composition of the viral vector may be directly injected into the lacrimal gland. The nicotinic acetylcholine receptor (nAChR) agonist is administered via local nasal administration in an effective amount. The viral vector includes a polynucleotide encoding a gene product, or functional variant thereof. The nAChR agonist may be provided as its pharmaceutically acceptable salt.

In various embodiments, the gene product is a therapeutic protein, antibody or antibody fragment (e.g., Fab), or a receptor decoy (e.g., soluble receptor, or “trap” molecules). In some embodiments, the therapeutic protein is nerve growth factor (NGF), glial-cell derived neurotrophic factor (GDNF), an RNase (e.g., ranpiranase), vascular endothelial growth factor (VEGF), sialic acid-binding Ig-like lectin 8 (SIGLEC-8), tumor necrosis factor (TNF-α), epidermal growth factor (EGF), interleukin-1 (IL-1), or interleukin-2 (IL-2), and interleukin-6 (IL-6). In some embodiments, the therapeutic protein is an antibody or antibody fragment. In some embodiments, the therapeutic protein is receptor decoy for IgE,

In embodiments that employ an adeno-associated virus (AAV) as the viral vector, the AAV may be of serotype 5 (AAV5), serotype 2 (AAV2), serotype 8 (AAV8), serotype 9 (AAV9). Various other serotypes or hybrids thereof may be used, such as a serotype that effectively transduces the target tissue (e.g., the lacrimal gland).

The viral vector may be provided at various concentrations, such as about 1×10⁷ genome copies per milliliter (GC/mL), about 5×10⁷ GC/mL, about 1×10⁸ GC/mL, about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, or about 6.2×10¹² GC/mL of the viral vector (e.g., AAV vector). About 100 μL of the composition may be administered, or other volumes.

In some embodiments, the nAChR agonist is varenicline, or pharmaceutically acceptable salts thereof (e.g., tartrate, salt). In some embodiments, the nAChR agonist is (R)-5-((E)-2-pyrrolidin-3-ylvinyl)pyrimidine, also known as simpanicline, 5-{(E)-2-[(3R)-pyrrolidin-3-yl]vinyl}pyrimidine, or (R,E)-5-((2-pyrrolidine-3-yl)vinyl)pyrimidine, or pharmaceutically acceptable salts thereof (e.g., hemi-galactarate or mono-citrate salts). Local nasal administration of the nAChR agonist may be via an intranasal spray or other suitable route.

Advantageously, the method results in expression of the gene product in the tear film; expression of gene product on or in the cornea of an eye of the subject; and/or an increased amount of the gene product delivered to the tear film in a predetermined time after administration of the treatment compared to administration of the viral vector alone. The predetermined time may be about 5 minutes, about 1 hour, about 1 day, about 1 week, or any time-frame suitable for detecting an increased amount or rate of delivery of the gene product to the tear film compared to a control (e.g., viral vector alone).

The disclosure further provides method of delivering a gene product to the ocular surface of a subject's eye. Variations and embodiments of this method include those described above for delivering a gene product to the tear film. Advantageously, the method results in expression of the gene product on the ocular surface; and/or an increased amount of the gene product delivered to the ocular surface in a predetermined time (e.g., about 5 minutes, about 1 hour, about 1 day, or about 1 week) compared to administration of the viral vector alone.

Further provided are viral vectors, pharmaceutical formulations, kits, and compositions for use in any of the methods disclosed herein.

Additional embodiments, features, and advantages of the disclosure will be apparent from the following detailed description and through practice of the disclosure.

In some embodiments, the disclosure provides a method of treating an ocular surface disorder, cornea disorder, or anterior chamber disorder in a subject, comprising:
a. administering an effective amount of a primary therapeutic agent to the eye or a tissue in fluidic communication with the eye; and
b. administering a treatment that increases tear production.

In some embodiments described herein, the treatment that increases tear production comprises an effective amount of a nicotinic acetylcholine receptor (nAChR) agonist, or a pharmaceutically acceptable salt thereof. In some embodiments, the nAChR agonist is a full agonist of nAChR subtypes alpha4beta2, alpha3beta4, alpha3alpha5beta4, and/or alpha4alpha6beta2. In some embodiments, the nAChR agonist is varenicline, or a pharmaceutically acceptable salt thereof. In some embodiments, the nAChR agonist is (R)-5-((E)-2-pyrrolidin-3-ylvinyl)pyrimidine, or a pharmaceutically acceptable salt thereof.

In some embodiments described herein, the treatment that increases tear production is administered via local nasal administration. In some embodiments, the local nasal administration is via an intranasal spray.

In some embodiments described herein, the primary therapeutic agent is administered via intralacrimal injection into one or both lacrimal glands or topical administration.

In some embodiments described herein, the treatment that increases tear production is administered subsequent to administration of the primary therapeutic agent. In some embodiments, the treatment that increases tear production is administered beginning 7 days subsequent to administration of the primary therapeutic agent.

In some embodiments described herein, the treatment that increases tear production is an effective electrical stimulation of the anterior ethmoid nerve.

In some embodiments described herein, the tissue in fluidic communication with the eye is one or any combination of the lacrimal gland, the lacrimal duct, the cornea, cornea epithelium, limbal stem cells, conjunctiva, lacrimal puncta, and lacrimal canaliculi. In some embodiments, the tissue in fluidic communication with the eye is the cornea. In some embodiments, tissue in fluidic communication with the eye is the lacrimal gland.

In some embodiments described herein, the method results an increased amount of the primary therapeutic agent delivered to the tear film of an eye of the subject in a predetermined time compared to administration of the primary therapeutic agent alone. In some embodiments, the method results in an increased amount of the primary therapeutic agent delivered to the cornea of an eye of the subject in a predetermined time compared to administration of the primary therapeutic agent alone.

In some embodiments, the predetermined time is about 5 minutes. In some embodiments, the predetermined time is about 1 hour.

In some embodiments described herein, administering an effective amount of the primary therapeutic agent comprises administering an effective amount of a composition comprising a viral vector comprising a polynucleotide encoding a gene product, or functional variant thereof, to at least one lacrimal gland of the subject, wherein the primary therapeutic agent comprises the gene product, or functional variant thereof. In some embodiments, the gene product is a therapeutic protein. In some embodiments, wherein the therapeutic protein is Nerve Growth Factor (NGF). In some embodiments, the therapeutic protein is Glial Derived Neurotrophic Factor (GDNF).

In some embodiments described herein, the viral vector is an adeno-associated virus (AAV) vector. In some embodiments, the viral vector is a vaccinia virus vector. In some embodiments, the viral vector is an adenovirus vector. In some embodiments, the viral vector is a lentiviral vector. In some embodiments, the AAV vector is an AAV of serotype 5 (AAV5), serotype 2 (AAV2), serotype 8 (AAV8), or serotype 9 (AAV9).

In some embodiments described herein, the composition comprises about 1×10¹² genome copies per milliliter (GC/mL) of the AAV vector.

In some embodiments described herein, the composition comprises about 6.2×10¹² genome copies per milliliter (GC/mL) of the AAV vector.

In some embodiments described herein, about 50 μL to about 100 μL of the composition is administered.

In some embodiments described herein, the method results in expression of the gene product in the tear film of an eye of the subject. In some embodiments, the method results in expression of the gene product in the cornea of an eye of the subject.

In some embodiments described herein, the method results an increased amount of the gene product delivered to the tear film in a predetermined time compared to administration of the viral vector alone. In some embodiments, the predetermined time is about 5 minutes. In some embodiments, the predetermined time is about 1 hour.

In some embodiments described herein, the ocular surface disorder, cornea disorder, or anterior chamber disorder in the subject is neurotrophic keratitis, chemical burn of the ocular surface, corneal wound, corneal ulcer, persistent epithelial defect, dry eye disease, herpes simplex viral infection of the trigeminal nerve and/or the eye, varicella zoster virus infection of the trigeminal nerve and/or the eye, or diabetic complications of the corneal nerves.

In some embodiments, the disclosure provides a method of delivering a gene product to the ocular surface at least one eye of a subject in need thereof, comprising:
a. administering an effective amount of a composition comprising a viral vector comprising a polynucleotide encoding a gene product, or functional variant thereof, to at least one lacrimal gland of the subject; and
b. administering via local nasal administration an effective amount of a nicotinic acetylcholine receptor (nAChR) agonist, or a pharmaceutically acceptable salt thereof.

In some embodiments described herein, the gene product is a therapeutic protein. In some embodiments, the therapeutic protein is Nerve Growth Factor (NGF). In some embodiments, the therapeutic protein is Glial Derived Neurotrophic Factor (GDNF)

In some embodiments described herein, the viral vector is an adeno-associated virus (AAV) vector. In some embodiments, the viral vector is a vaccinia virus vector. In some embodiments, the viral vector is an adenovirus vector. In some embodiments, the viral vector is a lentiviral vector. In some embodiments, the AAV vector is an AAV of serotype 5 (AAV5), serotype 2 (AAV2), serotype 8 (AAV8), or serotype 9 (AAV9).

In some embodiments described herein, the composition comprises about 1×10¹² genome copies per milliliter (GC/mL) of the AAV vector.

In some embodiments described herein, the composition comprises about 6.2×10¹² genome copies per milliliter (GC/mL) of the AAV vector.

In some embodiments described herein, about 50 μL to about 100 μL of the composition comprising a viral vector is administered.

In some embodiments described herein, the treatment that increases tear production is administered subsequent to administration of the primary therapeutic agent. In some embodiments, the treatment that increases tear production is administered beginning 7 days subsequent to administration of the primary therapeutic agent.

In some embodiments described herein, the method results in expression of the gene product in the tear film of an eye of the subject.

In some embodiments described herein, the method results in expression of the gene product in the cornea of an eye of the subject.

In some embodiments described herein, the method results an increased amount of the gene product delivered to the tear film in a predetermined time compared to administration of the viral vector alone. In some embodiments, the predetermined time is about 5 minutes. In some embodiments, the predetermined time is about 1 hour. In some embodiments, the method results an increased amount of the gene product delivered to the ocular surface in a predetermined time compared to administration of the viral vector alone. In some embodiments, the predetermined time is about 5 minutes. In some embodiments, the predetermined time is about 1 hour.

In some embodiments described herein, the nAChR agonist is a full agonist of nAChR subtypes alpha4beta2, alpha3beta4, alpha3alpha5beta4, and/or alpha4alpha6beta2. In some embodiments, the nAChR agonist is varenicline, or a pharmaceutically acceptable salt thereof. In some embodiments, the nAChR agonist is (R)-5-((E)-2-pyrrolidin-3-ylvinyl)pyrimidine, or a pharmaceutically acceptable salt thereof. In some embodiments, the nAChR agonist is administered via local nasal administration.

In some embodiments described herein, the composition comprising a viral vector is administered via intralacrimal injection.

In some embodiments described herein, the ocular surface disorder, cornea disorder, or anterior chamber disorder in the subject is neurotrophic keratitis, chemical burn of the ocular surface, corneal wound, corneal ulcer, persistent epithelial defect, dry eye disease, herpes simplex viral infection of the trigeminal nerve and/or the eye, varicella zoster virus infection of the trigeminal nerve and/or the eye, or diabetic complications of the corneal nerves.

In some embodiments, the disclosure provides a primary therapeutic agent and a treatment for increasing tear production for use in a method of treating an ocular surface disorder, cornea disorder, or anterior chamber disorder in a subject, the method comprising:
a. administering an effective amount of the primary therapeutic agent to the eye or a tissue in fluidic communication with the eye; and
b. administering the treatment that increases tear production.

In some embodiments, the disclosure provides a primary therapeutic agent and treatment for increasing tear production for use, the treatment that increases tear production comprises an effective amount of a nicotinic acetylcholine receptor (nAChR) agonist, or a pharmaceutically acceptable salt thereof. In some embodiments, the nAChR agonist is varenicline, or a pharmaceutically acceptable salt thereof. In some embodiments, the treatment that increases tear production is administered via local nasal administration.

In some embodiments described herein, administering an effective amount of the primary therapeutic agent comprises administering an effective amount of a composition comprising a viral vector comprising a polynucleotide encoding a gene product, or functional variant thereof, to at least one lacrimal gland of the subject, the primary therapeutic agent comprises the gene product, or functional variant thereof.

In some embodiments, the disclosure provides primary therapeutic agent and treatment for increasing tear production for use in the manufacture of a medicament for treatment of an ocular disease, disorder, or condition.

In some embodiments, the disclosure provides a kit comprising:
(a) a first composition comprising a viral vector comprising a polynucleotide encoding a gene product, or functional variant thereof; and
(b) a second composition comprising a nicotinic acetylcholine receptor (nAChR) agonist,
   and instructions for treating or delaying progression of a disease, disorder, or condition described herein in a subject in need thereof.

### BRIEF DESCRIPTION OF THE DRAWINGS

FIG. 1A shows an example of delivery of a viral vector to the lacrimal gland of a human subject.

FIG. 1B shows an example of delivery of a viral vector to the lacrimal gland of a human subject.

FIG. 2 shows a depiction of lacrimal gland physiology with respect to lacrimal acinar cells' secretion of expressed protein into tear film and tear induction via stimulation of the parasympathetic nerve pathway by nasal administration of a nerve stimulant.

FIG. 3 shows an example of the visual analog scale used in the Eye Dryness test for subjects to record the severity of their symptoms.

FIG. 4 shows a grading diagram of the division of the corneal surface based on the NEI/Industry Workshop scale.

FIG. 5A shows an illustrative vector encoding a recombinant AAV (rAAV) expression cassettes that includes regulatory elements and a human nerve growth factor (hNGF) transgene.

FIG. 5B shows an illustrative vector encoding a recombinant AAV expression cassettes that includes regulatory elements and a human glial-derived neurotrophic factor (hGDNF) transgene.

FIGS. 6A-6K are images of lacrimal tissue stained with anti-eGFP antibody. Lacrimal glands were dosed with rAAV virions containing an expression cassettes with eGFP transgene by intralacrimal injection. Lacrimal tissue was stained with anti-eGFP antibody to assess Egfp expression. Black arrows indicate staining showing eGFP expression.

FIGS. 7A-7E are images of lacrimal tissue stained with anti-hNGF antibody. Lacrimal glands were dosed with rAAV virions (AAV9 serotype) engineered to deliver an expression cassettes with hNGF transgene by intralacrimal injection. Lacrimal tissue was stained with anti-hNGF antibody to assess hNGF expression. Black arrows indicate staining showing hNGF expression.

FIG. 8 is a plot showing the concentration of hNGF in tear film in animals treated with AAV.hNGF and varenicline. Protein concentration was measured using Schirmer tear strips to collect tear film from Dutch-belted rabbits at the indicated number of days following intralacrimal administration of AAV2.hNGF, AAV5.hNGF, or AAV9.hNGF. At days 21, 28, and 41, tear film was collected following the intranasal administration of the tear inducing agent, varenicline. Control animals had no injection of AAV.

FIG. 9 shows an illustrative plasmid containing an rAAV expression cassette of SEQ ID NO: 25 with inverted terminal repeats (ITRs), a CAG promoter, and nerve growth factor (NGF) transgene polynucleotide elements.

FIGS. 10A-10C are plots showing concentration (pg/mL) of hNFGβ in tear film of pigs. Tears were collected from the left eye of pigs (n=4) on Day 7, 14, 21, 28, and 35 after transduction with an adeno-associated viral vector (AAV) encoding human nerve growth factor (AAV-hNGFβ). AAV2-hNGFβ (FIG. 10A) and AAV5-hNGFβ (FIG. 10B), had a detectable levels of hNGFβ within the standard curve range of the MSD assay 7 days after transduction. AAV9-hNGFβ (FIG. 10C) had tear levels of hNGFβ above the level of quantitation for the upper limit of the MSD assay 7 days after transduction.

FIG. 11 shows a schematic diagram depicting the elements between the ITRs of an AAV plasmid. The plasmid encodes EGFP linked to a secretion signal at its N-terminus (“secEGFP”) under control of a CMV promoter. The woodchuck hepatitis virus post-translational regulatory element (WPRE) serves to increase transgene expression and is proximal to the bovine growth hormone polyadenylation (pA) signal.

FIG. 12 provides images of porcine lacrimal gland that received an injection of AAV-secEGFP (AAV2 or AAV9 serotype) and was harvested on Day 103 and fixed in paraffin. IHC of 5 μM sections was performed using anti-GFP antibody and DAPI (nuclear) counterstain. Images were captured using a confocal microscope at 100× magnification. Negative control animals received no injection.

FIG. 13 provides an image of porcine lacrimal gland that received an injection of AAV9-secEGFP and was harvested on Day 103 and fixed in paraffin. IHC of 5 μM sections was performed using anti-GFP antibody and DAPI (nuclear) counterstain. In addition to lacrimal gland acinar cells, ductile epithelial cells appear to be transduced by AAV9 (white arrows).

FIG. 14 provides an image of porcine lacrimal gland that received an injection of AAV was harvested at Day 103 and fixed in paraffin. H&E staining of 5 μM paraffin sections at 100× reveal no inflammatory infiltrate, macro, or micro abnormalities.

### DETAILED DESCRIPTION

The inventors have recognized that increasing tear production in a subject improves medical treatment with various agents used in treatment of ocular surface disorders or anterior chamber disorders, such as without limitation, dry eye disease; bacterial, fungal, and viral keratitis; neurotrophic keratitis, blepharitis, Sjogren's syndrome; allergic, viral, and bacterial conjunctivitis; glaucoma; corneal neovascularization; pterygium; corneal dystrophies (keratoconus, Fuch's dystrophy, lattice dystrophy and map-dot-fingerprint dystrophy) and other ocular surface and cornea disorders. Further, the inventors have recognized that it is possible to recombinantly express gene product(s) such as protein(s) and/or nucleic acid(s) in the tissues surrounding the eye and that delivery of gene product(s) to the eye from surrounding tissues can be improved by stimulating tear production.

Medical treatment of the eye is being continuously improved by development of new therapeutic agents, including small-molecule and biologic drugs (termed herein, “therapeutic agent” or “primary therapeutic agent” to distinguish them from agents that stimulate tear production). Whereas previously, disorders such as dry eye and keratitis could only be treated with artificial tears (topical eye drops) used to moisten the eye, various medical treatments for ocular surface disorders or anterior chamber disorders have now been developed.

The primary therapeutic agents described herein, or others, are used in embodiments of this disclosure, e.g., to treat dry eye disease; bacterial, fungal, and viral keratitis; neurotrophic keratitis, blepharitis, Sjogren's syndrome; allergic, viral, and bacterial conjunctivitis; glaucoma; corneal neovascularization; pterygium; corneal dystrophies (keratoconus, Fuch's dystrophy, lattice dystrophy, corneal erosion syndrome, and map-dot-fingerprint dystrophy) and other ocular surface and cornea disorders.

The disclosure provides combination therapies and methods for improving medical treatment of ocular surface disorders or anterior chamber disorders by increasing tear production in the subject. As used herein, “subject” means any mammal, including mice, rabbits, non-human primates (NHP) and humans. In some embodiment, the subject is a human or NHP. Moreover, “individual” or “patient” may be used interchangeably with “subject.” Bioavailability and/or efficacy of one or more primary therapeutic agents is improved when tear production is increased by administration of an electrical stimulus, ultrasound stimulus, and/or a drug. The term “bioavailability” as used herein refers to the amount, distribution, and/or rate of delivery (e.g., in tear film) to the ocular surface—that is, to the pharmacokinetic properties of the agent independent of its effect on a disease state of the subject. For example, the amount, distribution, and/or rate of delivery of a primary therapeutic agent may be improved. The term “amount” as used herein refers to an absolute amount (e.g., an absolute amount of protein or rAAV particles) or concentration (e.g. a concentration of protein in a solution), whether the amount referred to in a given instance refers to an absolute amount, concentration, or both, will be clear to the skilled artisan based on the context provided herein. The term “efficacy” as used herein refers to an observed effect on a disease state in a subject. For example, efficacy in treatment of a dry eye disorder refers to a decrease in one or more symptoms of dry eye. Increased tear production may be particularly effective in improving delivery to the eye of gene product(s), such as protein(s) or nucleic acids, recombinantly expressed in tissues adjacent to the eye.

In particular embodiments, the disclosure provides methods of increasing the bioavailability and/or efficacy of a primary therapeutic agent by administering a treatment that increases tear production. In some embodiments, the treatment that increase tear production comprises administering an effective amount of a nicotinic acetylcholine receptor (nAChR) agonist, or a pharmaceutically acceptable salt thereof. In some embodiments, the treatment that increase tear production is electrical stimulation.

As used herein, “administer,” “administering,” “administration” and the like refers to providing a substance described herein to a subject in a manner that is pharmacologically useful (e.g., to treat a disease, disorder, or condition in the subject).

In particular embodiments, the disclosure provides methods of delivering a gene product to the tear film of a subject's eye by administering a viral vector (e.g., vaccinia, adenovirus, or adeno-associated virus) for expression of the gene product and a treatment that increases tear production and thereby increase the bioavailability and/or efficacy of the gene product.

Treatments that Increase Tear Production

### Electrical Stimulation

One treatment that increases tear production is electrical stimulation. For example, electrical stimulation of the anterior ethmoid nerve can increase aqueous tear volume, reduce tear osmolarity, add lipid, and increase the concentration of normal tear proteins. The anterior ethmoidal nerve can be stimulated using a neurostimulator implanted beneath the nasal mucosa or by an intranasal stimulator such as the TrueTear® Intranasal Tear Neurostimulator used according to the Professional Information Guide available at https://media.allergan.com. The nasal stimulator may be contacted to the inside of the nose at a depth of up to about 28 mm from the nasal columella, and then triggered to provide electrical stimulation. Illustrative stimulation methods are provided in Brinton et al. Investigative Ophthalmology & Visual Science (hereinafter IOVS) 58(4):2341-38.

### Vibratory Stimulation

In some embodiments, a treatment that increases tear production is oscillatory energy. In some embodiments, a treatment that increases tear production is oscillatory energy activated using the iTEAR®100. In some embodiments, a treatment that increases tear production is a vibratory stimulus. In some embodiments, the oscillatory energy or vibratory stimulus activates the external nasal nerve to increase tear production.

### Ultrasound Stimulation

Another treatment that increases tear production is ultrasound stimulation. For example, sound waves can be safely be applied to the eyelid of an eye of a patient or through the eyelid to other structures in the eye or to or through structures in the facial region to stimulate tear production, as described, e.g., in US 2018/0104514. In some embodiments, ultrasound is used to stimulate tear production by a lacrimal gland in the eye, e.g, after administration of a viral or non-viral vector to the lacrimal gland. For example, when exposed to direct skin vibration at approximately 100 Hz-300 Hz vibration produces tear formation bilaterally when just a single side is stimulated.

In some embodiments, ultrasound stimulation is administered with a neuromodulation device, like the iTear system (Olympic Ophthalmics) developed to treat dry eye disease.

### Tear-Stimulating Drugs

Another treatment that increases tear production is a tear-stimulating drug or a pharmaceutically acceptable salt thereof. As used herein, the term “treatment that increases tear production” excludes artificial tears (also known as topical eye drops) and refers only to treatments that increase tear production by the lacrimal gland of the subject.

Tear-stimulating drugs include cholinergics (pilocarpine, cevimeline), as well as nicotinic acetylcholine receptor (nAChR) agonists.

Nicotinic Acetylcholine Receptor (nAChR) Agonists

Further illustrative tear-stimulating drugs suitable for use in methods of the disclosure include a nicotinic acetylcholine receptor (nAChR) agonist or a pharmaceutically acceptable salt thereof. nAChRs are a class of pentameric ligand-gated ion channels that have high affinity and selectivity for both nicotine and acetylcholine (which resembles nicotine in its protonated form) and comprise combinations of alpha and beta subunits. Examples of nAChR subtypes include, but are not limited to, alpha3beta4, alpha4beta2, alpha3alpha5beta4, and alpha4alpha6beta2. An important nAChR receptor subtype involved in instigating the nasolacrimal reflex, for example, is the alpha4beta2 subtype located on the trigeminal nerve endings in the nasal mucosa.

Administration of a nAChR agonist may desensitize the receptor. Receptor desensitization results in reduced response to an agonist even at higher agonist concentrations, which further results in diminished efficacy of the treatment. For instance, short term desensitization of the nAChR receptor to an agonist may occur over a 24-hour period after administration of the agonist. The potential for receptor desensitization may potentially limit the dosing frequency over a period of time in order to preserve an effective response to the agonist.

A nAChR agonist may be characterized as a full or partial agonist as determined by its ability to activate a given receptor to produce a response as compared to the response at that receptor for acetylcholine (ACh). In general, a nAChR agonist is a full agonist if it evokes a response upon binding to a given receptor that is equal or greater to that of ACh. A nAChR agonist is a partial agonist if it evokes a lower response upon binding to the receptor as compared to the response generated from ACh.

nAChR agonist response, from which receptor activation can be determined can, for example, be generated using an appropriate cell-based assay. Cells designed to express a particular nAChR receptor subtype and generate an electrical current response when bound to and activated by a nAChR agonist can be used to characterize the agonist profile of a compound and the amount of receptor activation thus determined. An example of a generic protocol is described below.

Cells that express a particular human nAChR subtype are first exposed to ACh. ACh binds and activates the receptor, thereby evoking a current. The concentration of the ACh is chosen to elicit the maximum response of the receptor (e.g., 1280 micromolar ACh). This current is recorded as the ACh response and serves as the 100% nAChR agonist response and to which responses to other nAChR agonists are compared. After washing, the cells are exposed to a nAChR agonist at various concentrations (e.g., 0.1, 0.3, 1, 3, 10, 30, 100, and 300 micromolar). The current evoked by exposure to the nAChR agonist is measured and recorded for each nAChR concentration. This nAChR agonist response data is then normalized to unity versus the maximal ACh evoked current and plotted as a function of the logarithm of the nAChR agonist concentration. The nAChR agonist response is then calculated as a percentage of the ACh response.

In some cases, the method to determine the relative agonist activity of nAChR agonist comprises conditions wherein the ACh response is evoked from a 1 or more millimolar ACh solution.

A nAChR agonist evoking a response equal to or greater than the maximum ACh response determined at the same receptor type is a full agonist. In some cases, a nAChR agonist evoking a response of less than 100% of the ACh response may still be characterized a full agonist, taking into account experimental variability. For example, variability between tests or measurement methods, and statistical error, may account for differences in the response results. In some cases, a nAChR agonist evoking 80% to 120% of the ACh response is considered a full agonist. In some cases, a nAChR agonist evoking 99% of the ACh response or greater is considered a full agonist. In some cases, a nAChR agonist evoking 95% of the ACh response or greater is considered a full agonist. In some cases, a nAChR agonist evoking 90% of the ACh response or greater is considered a full agonist. In some cases, a nAChR agonist evoking 85% of the ACh response or greater is considered a full agonist. In some cases, a nAChR agonist evoking 80% of the ACh response or greater is considered a full agonist.

Taking into account experimental variability, if the nAChR agonist evokes less than 100% of the ACh response, then generally the agonist is considered a partial agonist. In some cases, a nAChR agonist evoking less than 95% of the ACh response is considered a partial agonist. In some cases, a nAChR agonist evoking less than 90% of the ACh response is considered a partial agonist. In some cases, a nAChR agonist evoking less than 85% of the ACh response is considered a partial agonist. In some cases, a nAChR agonist evoking less than 80% of the ACh response is considered a partial agonist.

In some cases, a nAChR agonist evoking 5% to 95% of the ACh response is considered a partial agonist. In some cases, a nAChR agonist evoking 5% to 90% of the ACh response is considered a partial agonist. In some cases, a nAChR agonist evoking 5% to 85% of the ACh response is considered a partial agonist. In some cases, a nAChR agonist evoking 5% to 80% of the ACh response is considered a partial agonist.

In some cases, a nAChR agonist evoking 10% to 95% of the ACh response is considered a partial agonist. In some cases, a nAChR agonist evoking 10% to 90% of the ACh response is considered a partial agonist. In some cases, a nAChR agonist evoking 10% to 85% of the ACh response is considered a partial agonist. In some cases, a nAChR agonist evoking 10% to 80% of the ACh response is considered a partial agonist.

nAChR agonists that generate a low level of electrical activity at relatively high concentrations of agonist may be described as a weak partial agonist. In some cases, a nAChR agonist evoking 30% or less of the ACh response is considered a weak partial agonist. In some cases, a nAChR agonist evoking 25% or less of the ACh response is considered a weak partial agonist. In some cases, a nAChR agonist evoking 20% or less of the ACh response is considered a weak partial agonist. In some cases, the relatively high concentration of nAChR agonist is at least 100 micromolar. In some cases, the relatively high concentration of nAChR agonist is at least 200 micromolar. In some cases, the relatively high concentration of nAChR agonist is at least 300 micromolar or greater. For instance, a 300 micromolar concentration of nAChR agonist that evokes 25% of the maximal Ach-evoked current is considered a weak partial agonist.

In some embodiments, the nAChR agonist is a full agonist. In some embodiments, the nAChR agonist is a partial agonist. In some embodiments, the nAChR agonist is a weak partial agonist.

In some embodiments, the nAChR agonist, or a pharmaceutically acceptable salt thereof, is an agonist of at least one of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of at least two of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of at least three of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of nAChR subtype alpha3beta4. In some embodiments, the nAChR agonist, or a pharmaceutically acceptable salt thereof, is an agonist of nAChR subtype alpha3alpha5beta4. In some embodiments, the nAChR agonist, or a pharmaceutically acceptable salt thereof, is an agonist of nAChR subtype alpha4beta2. In some embodiments, the nAChR agonist, or a pharmaceutically acceptable salt thereof, is an agonist of nAChR subtype alpha4alpha6beta2. In some embodiments, the nAChR agonist, or a pharmaceutically acceptable salt thereof, is an agonist of nAChR subtype alpha7. In some embodiments, the nAChR agonist, or a pharmaceutically acceptable salt thereof, is not an agonist of nAChR subtype alpha7. In some embodiments, the nAChR agonist, or a pharmaceutically acceptable salt thereof, is not a full agonist of nAChR subtype alpha7. In some embodiments, the nAChR agonist is a full agonist of the aforementioned subtypes. In some embodiments, the nAChR agonist is a partial agonist of the aforementioned subtypes. In some embodiments, the nAChR agonist is a weak partial agonist of the aforementioned subtypes.

The terms “alpha7” or “α7” nAChR refer to the homomeric alpha7 subtype, wherein the pentameric subunits of nAChR are composed entirely of alpha7 subunits. Thus, a nAChR agonist that binds and activates nAChR alpha7 is an agonist that binds and activates nAChR homomeric alpha7 receptor. In some embodiments described herein, the nAChR agonist is not an alpha7 receptor agonist.

In some embodiments, the nAChR agonist, or a pharmaceutically acceptable salt thereof, selectively binds to at least one of the nAChR subtypes selected from alpha3beta4, alpha3alpha5beta4, alpha4beta2, and alpha4alpha6beta2. As used herein, “selectively binds” or “is selective for” means that a compound has a higher affinity for the nAChR subtype and/or a lower half-maximal effective concentration (EC50) for that nAChr subtype for at least one reference nAChR subtype. Selectivity may be associated with at least a 5-fold affinity difference in EC50 value, at least a 10-fold affinity difference in EC50 value, at least a 20-fold affinity difference in EC50 value, or at least a 50-fold affinity difference in EC50 value. In some embodiments, the nAChR agonist, or a pharmaceutically acceptable salt thereof, selectively binds to nAChR subtype alpha3beta4. In some embodiments, the nAChR agonist, or a pharmaceutically acceptable salt thereof, selectively binds to nAChR subtype alpha3alpha5beta4. In some embodiments, the nAChR agonist, or a pharmaceutically acceptable salt thereof, selectively binds to nAChR subtype alpha4beta2. In some embodiments, the nAChR agonist, or a pharmaceutically acceptable salt thereof, selectively binds to nAChR subtype alpha4alpha6beta2. In some embodiments, the nAChR agonist, or a pharmaceutically acceptable salt thereof, selectively binds to nAChR subtype alpha7. In some embodiments, the nAChR agonist, or a pharmaceutically acceptable salt thereof, does not selectively bind to nAChR subtype alpha7.

The nAChR agonists contemplated in this disclosure include varenicline, a pharmaceutically acceptable salt thereof, and compound 1, or a pharmaceutically acceptable salt thereof. In some embodiments the nAChR agonist is not varenicline.

Varenicline is characterized as a full agonist of the nAChR subtype alpha7 and a partial agonist of subtypes alpha3beta4, alpha4beta2, alpha6beta2, alpha3alpha5beta4, and alpha4alpha6beta2. In some of the embodiments described herein, the nAChR agonist is varenicline, or a pharmaceutically acceptable salt thereof. Pharmaceutically acceptable salts of varenicline include varenicline tartrate. Additional related information for varenicline may be found in, for example, U.S. Pat. Nos. 9,504,644, 9,504,645, 9,532,944, 9,597,284, and 10,456,386.

Compound 1, as recited herein, refers to the structure:

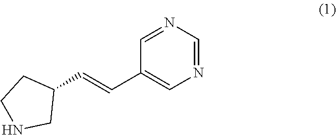 [SMILES: C(=C\[C@H]1CCNC1)/c1cncnc1]

An alternative structural representation of compound 1 is shown here:

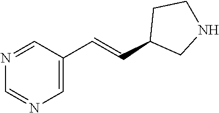 [SMILES: C(=C\[C@H]1CCNC1)/c1cncnc1]

Compound 1 may be also referred to by its chemical name. For instance, compound 1 is also referred to as (R)-5-((E)-2-pyrrolidin-3-ylvinyl)pyrimidine, or variations thereof including simpanicline, 5-{(E)-2-[(3R)-pyrrolidin-3-yl]vinyl}pyrimidine and (R,E)-5-((2-pyrrolidine-3-yl)vinyl)pyrimidine.

Compound 1 is a full agonist of nAChR subtypes alpha4beta2, alpha3beta4, alpha3alpha5beta4, and alpha4alpha6beta2. Compound 1 is a full agonist of nAChR subtypes alpha4beta2, and alpha3beta4.

Compound 1 is a partial agonist of subtype alpha3beta2.

Compound 1 is a weak partial agonist of subtype alpha7. In one example, a 300 micromolar concentration of compound 1 citrate evoked only 25% of the maximal ACh-evoked current.

In some of the embodiments described herein, the nAChR agonist may be compound 1, or a pharmaceutically acceptable salt thereof. Pharmaceutically acceptable salts of compound 1 include galactarate (e.g., hemi-galactarate dihydrate) and citrate (e.g., mono-citrate). Patent related information for compound 1 may be found in U.S. Pat. Nos. 7,098,331, 7,714,001, 8,063,068, 8,067,443, 8,604,191, 9,145,396, 9,981,949, 8,633,222, and PCT publication WO 2017/177024.

In some of the embodiments described herein, the nAChR agonist is (R)-5-((E)-2-pyrrolidin-3-ylvinyl)pyrimidine, or a pharmaceutically acceptable salt thereof. In some of the embodiments described herein, the nAChR agonist is (R)-5-((E)-2-pyrrolidin-3-ylvinyl)pyrimidine hemigalactarate dihydrate. In some of the embodiments described herein, the nAChR agonist is (R)-5-((E)-2-pyrrolidin-3-ylvinyl)pyrimidine mono-citrate.

### Positive Allosteric Modulator

In some embodiments, the nAChR agonist is administered in conjunction with a positive allosteric modulator (PAM), or a pharmaceutically acceptable salt thereof. PAMs may bind to an allosteric site on the nAChR and positively allosterically modulate the receptor's activity in the presence of the physiological ligand, acetylcholine (ACh), or another nAChR agonist described herein, or and/or act alone as agonists or antagonists of the receptor through the allosteric binding site. In some embodiments, the PAMs do not act alone as agonists and/or antagonists of nAChR. The methods described herein, provide that the efficacy of nAChR agonists can be surprisingly improved by combining a nAChR agonist with a nAChR PAM. Such combinations are highly efficient for improving the efficacy of a nAChR agonist for treatment of dry eye disease, ocular discomfort, or increasing tear production, when compared to administration of a nAChR agonist alone.

In some of the embodiments described herein, the PAM is selected from the group consisting of 17-beta-Estradiol, Br-PBTC, ivermectin, galantamine, genistein, 5-hydroxyindole, 4BP-TQS, A-86774, CCMI, levamisole, morantel, LY-2087101, mecamylamine, menthol, NS206, NS1738, NS9283, PNU-120596, RO5126946, TBS-345, dFBR, and HEPES, or a pharmaceutically acceptable salt of any of the foregoing. The chemical name represented by each of the code name is recited below, and may be used interchangeably herein.

| | |
|---|---|
| Br-PBTC | (R)-7-bromo-N-(piperidin-3-yl)benzo[b]thiophene-2- |
| | carboxamide |
| NS9283 | 3-[3-(3-Pyridinyl)-1,2,4-oxadiazol-5-yl]benzonitrile |
| 4BP-TQS | 4-(4-Bromophenyl)-3a,4,5,9b-tetrahydro-3H- |
| | cyclopenta[c]quinoline-8-sulfonamide |
| A-86774 | 4-[5-(4-Chlorophenyl)-2-methyl-3-(1-oxopropyl)-1H- |
| | pyrrol-1-yl]benzenesulfonamide |
| CCMI | [N-(4-Chlorophenyl)]-α-[(4-chlorophenyl)- |
| | aminomethylene]-3-methyl-5-isoxazoleacetamide |
| LY-2087101 | [2-[(4-Fluorophenyl)amino]-4-methyl-5- |
| | thiazolyl]-3-thienylmethanone |
| NS206 | 3-N-Benzyloxy-3-hydroxyimino-2-oxo-6,7,8,9- |
| | tetrahydro-1H-benzo[g]indole-5-sulfonamide |
| NS1738 | N-(5-Chloro-2-hydroxyphenyl)-N′-[2-chloro-5- |
| | (trifluoromethyl)phenyl]urea |
| PNU-120596 | N-(5-Chloro-2,4-dimethoxyphenyl)-N′-(5-methyl-3- |
| | isoxazolyl)-urea |
| RO5126946 | (5-chloro-N-[(1S,3R)-2,2-dimethyl-3-(4-sulfamoyl- |
| | phenyl)-cyclopropyl]-2-methoxy-benzamide) |
| TBS-345 | TBS-345, 4-(3-(4-bromophenyl)-5-phenyl-1H-1,2,4- |
| | triazol-1-yl)benzenesulfonamide |
| dFBR | 6-Bromo-2-(1,1-dimethyl-2-propeny1)-N-1H-indole-3- |
| | ethanamine hydrochloride |
| HEPES | 4-(2-Hydroxyethyl)piperazine-1-ethanesulfonic acid |

In some of the embodiments described herein, the PAM is (R)-7-bromo-N-(piperidin-3-yl)benzo[b]thiophene-2-carboxamide (Br-PBTC) or 3-[3-(3-Pyridinyl)-1,2,4-oxadiazol-5-yl]benzonitrile (NS9283), or a pharmaceutically acceptable salt of either of the foregoing.

In some of the embodiments described herein, the PAM is (R)-7-bromo-N-(piperidin-3-yl)benzo[b]thiophene-2-carboxamide (Br-PBTC), or a pharmaceutically acceptable salt thereof. In some of the embodiments described herein, the PAM is 3-[3-(3-Pyridinyl)-1,2,4-oxadiazol-5-yl]benzonitrile (NS9283), or a pharmaceutically acceptable salt thereof.

### Primary Therapeutic Agents

In various embodiments, the methods of the disclosure comprise administering a primary therapeutic agent. The methods of the disclosure are well suited for use with primary therapeutic agents whose distribution in the eye is limited by rate of diffusion or other biophysical parameters. Such primary therapeutic agents may include large molecules (proteins and the like) whose diffusion may be limited by steric effects. However, bioavailability and/or efficacy of small molecule drugs may be improved, particularly when binding to particular tissues and/or molecules in the eye retards distribution of the small molecule drug. In other cases, the methods of the disclosure increase discharge of a primary therapeutic agent delivered to, implanted in, or expressed in (such as with a vector) a particular gland (e.g., the lacrimal gland). Thus, delivery of the primary therapeutic agent to the eye and its distribution over the ocular surface may both be increased. In some embodiments, the primary therapeutic agent is a biologic drug. In some embodiments, the primary therapeutic agent is a small-molecule drug. In some embodiments, the primary therapeutic agent is a nucleic acid (e.g., DNA or RNA). RNAs include ribozymes, interfering RNAs, small-hairpin RNAs, agomirs, and antagomirs. DNAs include plasmid DNA and other forms of DNA. The nucleic acid may be delivered as a naked polynucleotide or encapsulated in a non-viral vector. In some embodiments, the primary therapeutic agent is a nucleic-acid vector, such as a non-viral vector (liposome, nanoparticle, or the like) or a viral vector.

In some embodiments, the primary therapeutic agent used in methods of the disclosure is cyclosporine (marketed as RESTASIS, ZYCLORIN, or CYCLOKAT); lifitegrast (XIIDRA); immunomodulators (voclosporin, tacrolimus, pimecrolimus); Janus kinase (JAK)3 inhibitors (tasocitinib); secretagogues or mucoprotective agents (diquafosol, rebamipide, ecabet); disaccharide protectants (trehalose); nonsteroidal anti-inflammatory drugs (bromfenac); steroids (dexamethasone, EGP-437, corticosteroids, prednisolone, methylprednisolone, fluorometholone, lotemax and loteprednol containing agents); steroid-sparing drugs (mycophenolate, azathioprine, cyclophosphamide); fatty acids (omega-3 fatty acids, resolvin E1); broad-spectrum antimicrobial (fluoroquinolones), anti-viral or anti-fungal agents; antibiotics (tetracycline and its derivatives (doxycycline), macrolide antibiotic (azithromycin); autologous serum; topical vitamin A; and mast cell stabilizers (e.g., nedocromil, lodoxamide), antihistamines (e.g., emedastine, loratidine, chlorphenamine), or combined mast cell stabilizers/antihistamine (e.g., olopatadine). In some embodiments, the primary therapeutic agent used in the methods of the disclosure is a prostaglandin analog, beta blocker, alpha agonist, carbonic anhydrase inhibitor, or rho kinase inhibitor.

Other illustrative therapeutic agents used in methods of the disclosure include: topical albumin; nerve growth factor; insulin or insulin-like growth factor 1 (IGF-1); growth factors (hepatocyte growth factor, epidermal growth factor (EGF), transforming growth factor (TGF)-beta); fibronectin; inhibitors of pro-inflammatory cytokine pathways, including but not limited to an Interleukin-1 (IL-1) pathway inhibitor, e.g., anakinra or EBI-005; IL-6 pathway inhibitors, such as IL-6R antagonist; Tumor necrosis factor-α (TNF-α) inhibitors, such as etanercept (Enbrel), infliximab (Remicade), adalimumab (Humira), certolizumab pegol (Cimzia), golimumab (Simponi) and ESBA105; inhibitors of other pro-inflammatory cytokine pathways including IL-12 or IL-23, IFN-γ and IL-17 and their receptors; anti-inflammatory cytokines, such as IL-4 and IL-13; inhibitors of matrix metalloproteinases, such as tissue inhibitors of metalloproteinases (TIMPs); inhibitors of proinflammatory chemokine pathways, such as antagonist to the chemokine receptor 2 (CCR2); immune globulins and antibodies against anti-citrullinated protein autoantibodies (ACPA); silk protein and fibroin derivatives (e.g., SDP-4 by SilkTech Biopharmaceuticals); FK506 binding proteins (FK506BP) or their derivatives (e.g., PEP-1-FK506BP); biologics targeting T-cells, such as anti-CD4 monoclonal antibody (Arthritis Rheum. 2004 September; 50(9):2903-10); biologics targeting B-cells, such as rituximab, belimumab and epratuzumab; mucin family proteins, such as proteoglycan 4 (prg4) or a lubricating fragment thereof; hyaluronan synthase (HA synthase); lacritin and its derivatives; antagonists of proteins lymphocyte function-associated antigen-1 (LFA-1) and intercellular adhesion molecule-1 (ICAM-1); antiangiogenic agents, such as antagonists of vascular endothelial growth factors (VEGFs) and corresponding VEGF receptors (VEGFRs) (e.g., aflibercept, ranibizumab, bevacizumab and MP0112); elastin-like peptide; SLURP1 polypeptide or its derivatives; galectins and their modulators.

In some embodiments, the primary therapeutic agent is a recombinant human Nerve Growth Factor (rhNGF). In some embodiments, the disclosure provides methods of expressing rhNGF in the lacrimal gland using a vector (e.g., an AAV vector) and administering an agent that increase tear production (e.g., electrical stimulation; or an nAChR agonist such as varenicline, (R)-5-((E)-2-pyrrolidin-3-ylvinyl)pyrimidine, 5-{(E)-2-[(3R)-pyrrolidin-3-yl] vinyl}pyrimidine, or (R,E)-5-((2-pyrrolidine-3-yl)vinyl)pyrimidine, or pharmaceutically acceptable salts thereof).

In some embodiments, the primary therapeutic agent is Thymosin beta 4 (Tβ4). Tβ4 is a 43-amino acid peptide (having sequence SDKPDMAEIEKFDKSKLKKTETQEKNPLPSK ETIEQEKQAGES; SEQ ID NO: 26) that is a major constituent protein of platelets, macrophages, and polymorphonuclear cells, where it acts as a G-actin binding molecule and regulator of actin polymerization. In a Phase II clinical trial, a 28-day course of 0.1% Tβ4 ophthalmic formulation elicited significant positive effects on ocular discomfort and on corneal staining in subjects with dry eye. Sosne G, Ousler G W (2015) Clin Ophthalmol. 20:877-84.

Other illustrative primary therapeutic agents used in methods of the disclosure include: modulators of Integrin α4β1 (VLA-4).

The primary therapeutic agent may be administered to the eye by any suitable method, including but not limited to: topical administration in an aqueous solution, gel, or suspension, by injection into periocular tissue, or by injection into a gland (e.g., lacrimal gland).

The present disclosure provides methods of administering a viral or non-viral vector as the primary therapeutic agent to deliver a polynucleotide encoding a gene product (e.g., protein or nucleic acid). As used herein, the term “primary therapeutic agent” includes a viral or non-viral vector that delivers a polynucleotide encoding a gene product (e.g., protein) capable of having therapeutic effect on the eye. Similarly stated, the primary therapeutic agent may exert its effect indirectly by, e.g., causing the expression from the tissues of the subject of a gene product that has a desired biological effect when expressed and distributed to and across the ocular surface in an effective amount. An “effective amount,” as used herein, refers to an amount or dose of an rAAV, treatment, or composition described herein that is sufficient to reduce the symptoms and or signs of an ocular condition described herein.

Protein- and nucleic acid-based primary therapeutic agents known in the art can be converted to genetic vectors and thereby expressed in situ for delivery to the ocular surface and/or the cornea. Thus, for example and without limitation, the viral or non-viral vectors of the disclosure may comprise a polynucleotide encoding one or more of albumin; nerve growth factor; insulin or insulin-like growth factor 1 (IGF-1); growth factors (hepatocyte growth factor, epidermal growth factor (EGF), transforming growth factor (TGF)-beta); fibronectin; inhibitors of pro-inflammatory cytokine pathways, including but not limited to an Interleukin-1 (IL-1); pathway inhibitor, e.g., anakinra or EBI-005; IL-6 pathway inhibitors, such as IL-6R antagonist; Tumor necrosis factor-α (TNF-α) inhibitors, such as etanercept (Enbrel), infliximab, adalimumab, certolizumab, golimumab and ESBA105; inhibitors of other pro-inflammatory cytokine pathways including IL-12 or IL-23, IFN-γ and IL-17 and their receptors; anti-inflammatory cytokines, such as IL-4 and IL-13; inhibitors of matrix metalloproteinases, such as tissue inhibitors of metalloproteinases (TIMPs); inhibitors of proinflammatory chemokine pathways, such as antagonist to the chemokine receptor 2 (CCR2); immune globulins and antibodies against anti-citrullinated protein autoantibodies (ACPA); silk protein and fibroin derivatives (e.g., SDP-4 by SilkTech Biopharmaceuticals); FK506 binding proteins (FK506BP) or their derivatives (e.g., PEP-1-FK506BP); biologics targeting T-cells, such as anti-CD4 monoclonal antibody (Arthritis Rheum. 2004 September; 50(9):2903-10); biologics targeting B-cells, such as rituximab, belimumab and epratuzumab; mucin family proteins, such as proteoglycan 4 (prg4) or a lubricating fragment thereof; hyaluronan synthase (HA synthase); lacritin and its derivatives; antagonists of proteins lymphocyte function-associated antigen-1 (LFA-1) and intercellular adhesion molecule-1 (ICAM-1); antiangiogenic agents, such as antagonists of vascular endothelial growth factors (VEGFs) and corresponding VEGF receptors (VEGFRs) (e.g., aflibercept, ranibizumab, bevacizumab and MP0112); elastin-like peptide; SLURP1 polypeptide or its derivatives; galectins and their modulators.

In some embodiments, the viral or non-viral vector comprises a polynucleotide encoding a recombinant human Nerve Growth Factor (rhNGF). In various embodiments, the viral or non-viral vector comprises a polynucleotide encoding a therapeutic protein, antibody or antibody fragment (e.g., Fab), or a receptor decoy (e.g., soluble receptor, or “trap” molecules). In some embodiments, the viral or non-viral vector comprises a polynucleotide encoding nerve growth factor (NGF), glial-cell derived neurotrophic factor (GDNF), an RNase (e.g. ranpiranase), vascular endothelial growth factor (VEGF), sialic acid-binding Ig-like lectin 8 (SIGLEC-8), tumor necrosis factor (TNF-α), epidermal growth factor (EGF), interleukin-1 (IL-1), or interleukin-2 (IL-2), and interleukin-6 (IL-6). In some embodiments, the therapeutic protein is an enzyme. In some embodiments, the therapeutic antibody is a variable new antigen receptor (VNAR) antibody. In some embodiments, the therapeutic antibody is an immunoglobulin new antigen receptor (IgNAR) antibody. In some embodiments, the viral or non-viral vector comprises a polynucleotide encoding an antibody or antibody fragment. In some embodiments, the viral or non-viral vector comprises a polynucleotide encoding etanercept, infliximab, adalimumab, certolizumab, golimumab or ESBA105. In some embodiments, the viral or non-viral vector comprises a polynucleotide encoding a receptor decoy for IgE. In some embodiments, the viral or non-viral vector comprises a polynucleotide encoding thymosin beta 4 (Tβ4).

### Viral Vectors

Therapeutic use of polynucleotide vectors may be improved by the methods of the disclosure. The protein drugs (biologic), including growth factors and monoclonal antibodies, described herein may optionally be delivered to the eye in a polynucleotide vector (e.g., non-viral or viral vector). In some embodiments, a viral vector is delivered to the lacrimal gland. Vaccinia, adenovirus, and adeno-associated virus (AAV) vectors transduce primary cultures of lacrimal gland cells and/or lacrimal gland tissues. These and other viral vectors may be engineered for expressing in tissue adjacent to or in fluidic connection to the eye by selection of suitable promoter or enhance elements, selection of serotypes that transduce a selected tissue, or pseudotyping of the virus.

Viral vector-based delivery of transgenes encoding a protein product by injection into the lacrimal gland (i.e., intralacrimal delivery or intralacrimal administration) can be advantageous for treating ocular diseases and symptoms. Proteins produced in the lacrimal gland and secreted in the lacrimal fluid of a subject may be improved due to post-translational modification (PTM) (covalent and generally enzymatic modification of proteins following protein biosynthesis) whereby the proteins are synthesized by ribosomes translating mRNA into polypeptide chains, which may then undergo PTM to form the mature protein product. PTMs may confer important properties in cell signaling that would otherwise be absent with topical delivery of recombinant human proteins that are often made in bacteria such as Escherichia coli. In addition, when secreted with natural tear film components that may contain co-factors, chaperones, enzymes or other proteins, administration via the lacrimal fluid may confer biologic activity that could not be achieved with only administration of topically administered protein alone.

In some embodiments, the methods of the disclosure comprising administering a viral vector. The term “viral vector” as used herein refers to the viral particle having a polynucleotide encapsulated either in a viral capsid or a viral envelop and having one or more viral proteins sufficient to transduce target cells. The term viral vector includes replicating and non-replicating vectors and integrating and non-integrating vectors. Any viral vector capable of transducing cells of a tissue surrounding the eye or can be used. The viral vector may transduce cells of the subject locally or systemically provided that transduction leads to expression of a gene product onto the ocular surface. Delivery to the ocular surface may include direct secretion from cells onto the ocular surface, transcytosis of the gene product to the ocular surface, or localization to the ocular surface via the circulatory system. In particular embodiments, the method of the disclosure comprising administering the viral vector to the lacrimal gland. The lacrimal gland is in fluidic connection to the eye in that its physiological function is production of tears that are secreted onto the eye.

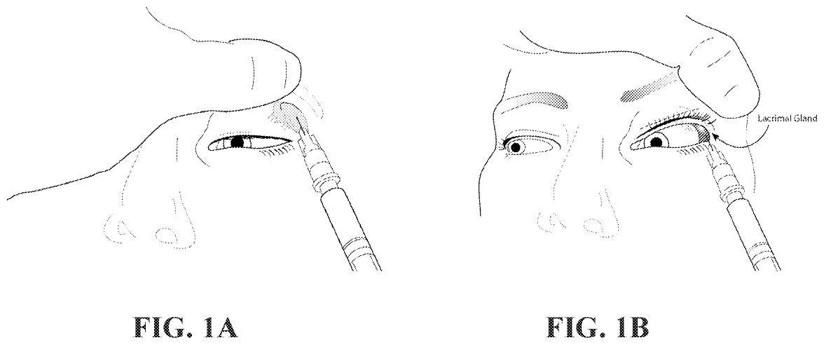

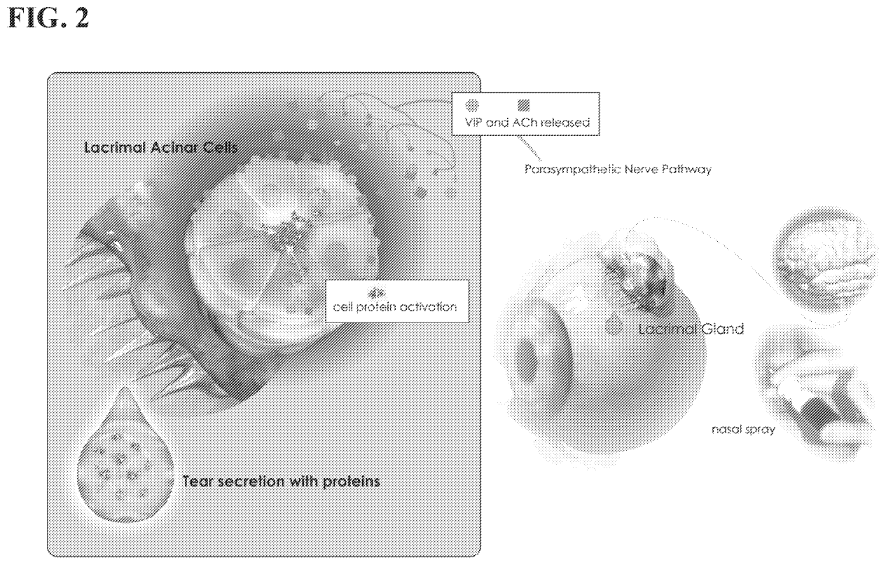

The “lacrimal gland” refers to other or both of the paired, almond-shaped exocrine glands (one for each eye) that secrete the aqueous layer of the tear film. The lacrimal glands are situated in the upper lateral region of each orbit, in the lacrimal fossa of the orbit formed by the frontal bone. Administration of a viral vector to the lacrimal gland may be accomplished by topical administration to the ocular surface, direct injection into the lacrimal gland, and/or topical administration to the lacrimal gland. The lacrimal gland may be accessed surgically or by manipulation of the eyelid. Manipulation of the eyelid provides access to the tissue for administration topically (e.g., by lavage of the tissue with a pharmaceutical composition comprising the viral vector). Direct injection into the lacrimal gland may be done by penetrating the skin over the lacrimal gland (FIG. 1A) or by manipulating the eyelid to access the lacrimal gland (FIG. 1B). The lacrimal gland is made up partially by acinar cells that express and secrete proteins into tear film (FIG. 2). In some embodiments, the rAAV is administered to the lacrimal gland by direct injection as depicted in FIG. 1A. In some embodiments, the rAAV is administered to the lacrimal gland by manipulating the eyelid as depicted in FIG. 1B.

In various embodiments, the viral vector is retrovirus (e.g., lentivirus), a vaccinia virus, a poxvirus, an adenovirus, or an adeno-associated virus (AAV). Standard molecular biology techniques exist for generating recombinant forms of each of these viruses that are suitable for use as vectors. Viral vectors include pseudotyped vectors (e.g., lentiviral vectors having Env proteins from VSV G or other viruses). In some embodiments, the viral vector is an AAV vector. AAV vectors include self-complementary and non-self-complementary vectors. One or more AAV vectors can be administered concurrently or sequentially. In some cases, a split vector system is used to deliver a large gene to a cell. For example, due to the ˜4.5 kb packaging limit of conventional AAV vectors, genes of about 4 kb up to about 10 kb may be split into two AAV vectors configured such that recombination within transduced cells produced a polynucleotide containing the full gene. Three or more AAV vectors may be used for larger genes. The term “gene” reference herein to a DNA polynucleotide encoding a gene product either directly (where the gene product is an RNA) or through a messenger RNA intermediate (where the gene product is a protein). The term “gene product” as used herein refers to the desired product expressed from the gene when the vector is transduced into the cell. In various embodiments, the gene product is a therapeutic protein, antibody or antibody fragment, or a receptor decoy. The term “therapeutic” as used herein denotes a gene product capable of producing a therapeutic effect on the eye when present on the ocular surface in an effective concentration for a sufficient period of time.

Gene products useful in the methods of the present disclosure are secreted by transduced cells to permit them to be delivery to the ocular surface and/or the tear film of the eye. In some embodiments, the gene product is an RNA. In some embodiments, the gene product is a therapeutic protein. In some embodiments, the therapeutic protein is nerve growth factor (NGF), glial-cell derived neurotrophic factor (GDNF), an RNase (e.g., ranpiranase), or sialic acid-binding Ig-like lectin 8 (SIGLEC-8). In some embodiments, the gene product comprises an antibody or an antigen-binding fragment thereof, either as the full-length protein gene product or as a portion of the protein gee product. In some embodiments, the antibody is a monoclonal antibody or a multispecific antibody (e.g., bispecific antibody). In some embodiments, the antibody is a single-domain antibody (sdAb) (i.e., nanobody).

Thus, for example and without limitation, the viral vectors of the disclosure may comprise a polynucleotide encoding one or more of albumin; nerve growth factor; insulin or insulin-like growth factor 1 (IGF-1); growth factors (hepatocyte growth factor, epidermal growth factor (EGF), transforming growth factor (TGF)-beta); fibronectin; inhibitors of pro-inflammatory cytokine pathways, including but not limited to an Interleukin-1 (IL-1); pathway inhibitor, e.g., anakinra or EBI-005; IL-6 pathway inhibitors, such as IL-6R antagonist; Tumor necrosis factor-α (TNF-α) inhibitors, such as etanercept, infliximab, adalimumab, certolizumab, golimumab and ESBA105; inhibitors of other pro-inflammatory cytokine pathways including IL-12 or IL-23, IFN-γ and IL-17 and their receptors; anti-inflammatory cytokines, such as IL-4 and IL-13; inhibitors of matrix metalloproteinases, such as tissue inhibitors of metalloproteinases (TIMPs); inhibitors of proinflammatory chemokine pathways, such as antagonist to the chemokine receptor 2 (CCR2); immune globulins and antibodies against anti-citrullinated protein autoantibodies (ACPA); silk protein and fibroin derivatives (e.g., SDP-4 by SilkTech Biopharmaceuticals); FK506 binding proteins (FK506BP) or their derivatives (e.g., PEP-1-FK506BP); biologics targeting T-cells, such as anti-CD4 monoclonal antibody (Arthritis Rheum. 2004 September; 50(9):2903-10); biologics targeting B-cells, such as rituximab, belimumab and epratuzumab; mucin family proteins, such as proteoglycan 4 (prg4) or a lubricating fragment thereof; hyaluronan synthase (HA synthase); lacritin and its derivatives; antagonists of proteins lymphocyte function-associated antigen-1 (LFA-1) and intercellular adhesion molecule-1 (ICAM-1); antiangiogenic agents, such as antagonists of vascular endothelial growth factors (VEGFs) and corresponding VEGF receptors (VEGFRs) (e.g., aflibercept, ranibizumab, bevacizumab and MP0112); elastin-like peptide; SLURP1 polypeptide or its derivatives; galectins and their modulators.

In some embodiments, the viral vector comprises a polynucleotide encoding a recombinant human Nerve Growth Factor (rhNGF). In various embodiments, the viral vector comprises a polynucleotide encoding a therapeutic protein, antibody or antibody fragment (e.g., Fab), or a receptor decoy (e.g., soluble receptor, or “trap” molecules). In some embodiments, the viral vector comprises a polynucleotide encoding nerve growth factor (NGF), glial-cell derived neurotrophic factor (GDNF), an RNase (e.g., ranpiranase), vascular endothelial growth factor (VEGF), sialic acid-binding Ig-like lectin 8 (SIGLEC-8), tumor necrosis factor (TNF-α), epidermal growth factor (EGF), interleukin-1 (IL-1), or interleukin-2 (IL-2), and interleukin-6 (IL-6). In some embodiments, the viral vector comprises a polynucleotide encoding an antibody or antibody fragment. In some embodiments, the viral vector comprises a polynucleotide encoding etanercept, infliximab, adalimumab, certolizumab, or golimumab. In some embodiments, the viral vector comprises a polynucleotide encoding a receptor decoy for IgE. In some embodiments, the viral vector comprises a polynucleotide encoding thymosin beta 4 (Tβ4).

### Expression Cassette

In some embodiments, the AAV viral vector is a recombinant AAV viral vector (rAAV). In some embodiments, the rAAV comprises an expression cassette and a capsid protein.

The rAAV virions of the disclosure may comprise an expression cassette. As used herein, the term “expression cassette” refers to a polynucleotide comprising at least one polynucleotide sequence encoding a protein of interest or transgene, e.g., a neurotrophic factor, flanked by inverted terminal repeats. The expression cassette may further comprise other polynucleotide sequences, e.g., promoters, regulatory elements (e.g., one or more promoters or enhancers), translation initiation sequences, coding sequences, and termination sequences. Regulatory elements may be operably linked to the transgene and promote expression.

In some embodiments, the expression cassette of the present disclosure comprises a polynucleotide sequence encoding a transgene product. In some embodiments, the transgene product is a protein that can treat an eye disease or disorder. For example, the transgene may be a protein responsible for maintenance or homeostasis of the eye, e.g., a neurotrophic factor. In some embodiments, the expression cassette provides increased expression of the transgene in the at least one eye and/or lacrimal glands. In some embodiments, expression of the transgene may be increased 5%, 10%, 15%, 20%, or 25% compared to expression of a natively expressed gene in an untreated subject, or in the contralateral eye of a treated subject. In some embodiments, expression of the transgene may be increased 1-fold, 2-fold, 3-fold, 4-fold, or 5-fold compared to expression of a natively expressed gene in an untreated subject, or in the contralateral eye of a treated subject. In some embodiments, expression of the neurotrophic factor is increased 5%, 10%, 15%, 20%, 25%, 30%, 35%, 40%, 45%, 50%, 55%, 60%, 65%, 70%, 75%, 80%, 85%, 90%, 95%, or 99% compared to expression of the neurotrophic factor in an untreated subject, or in the contralateral eye of a treated subject. In some embodiments, expression of the neurotrophic factor may be increased 1-fold, 2-fold, 3-fold, 4-fold, or 5-fold, 6-fold, 7-fold, 8-fold, or 9-fold compared to expression of the neurotrophic factor in an untreated subject, or in the contralateral eye of a treated subject. In some embodiments, the transgene may be expression at any detectable level in the treated eye, whereas the transgene may not be expressed, or expressed at undetectable levels, in an untreated subject, or in the contralateral eye of a treated subject. Put another way, the eye or lacrimal gland to which the rAAV virion is administered may express a transgene in higher abundance than in an eye or lacrimal gland that has only endogenous (i.e., native) expression of the transgene or in eyes that have a lower or impaired secretion of endogenous (i.e., native) expression of the transgene.

In some embodiments, the transgene is a neurotrophic factor. In some embodiments, the neurotrophic factor is nerve growth factor (NGF) protein or a functional variant thereof. As used herein, “NGF”, “NGFβ”, or “Nerve Growth Factor Beta” refer to Nerve Growth Factor which is a gene that encodes a protein which homodimerizes and performs nerve growth stimulating activity. NGF is also involved in the regulation and differentiation of neurons. As used herein, the term “NGF protein” refers to an NGF protein from any species. The term “functional variant” refers to variants having sequence substitutions, insertions, deletions, and/or N- or C-terminal truncations, where the functional variant retains one or more functions of the reference protein, e.g., a native NGF protein. NGF is a 26-kDa polypeptide involved primarily in the growth, maintenance, proliferation, and survival of nerve cells. In the cornea, NGF binds its receptor trkA^{NGFR} and p75^{NTR}, eliciting signal transduction pathways associated with healing of both cornea and conjunctiva (Micera et al., Exp Eye Res. 83:747-57 (2006)). In some embodiments, the NGF protein comprises SEQ ID NO. 1. In some embodiments, the NGF protein comprises a sequence at least 80%, 85%, 90%, 95%, 96%, 97%, 98%, 99%, or 100% identical to SEQ ID NO: 1. In some embodiments, the NGF protein shares a sequence with at least 80%, at least 85%, at least 90%, at least 95%, at least 96%, at least 97%, at least 98%, at least 99%, or 100% identity to SEQ ID NO: 1. In some embodiments, the polynucleotide encoding the NGF protein comprises SEQ ID NO: 2. In some embodiments, the polynucleotide encoding the NGF protein comprises a sequence at least 80%, 85%, 90%, 95%, 96%, 97%, 98%, 99%, or 100% identical to SEQ ID NO: 2. In some embodiments, the polynucleotide encoding the NGF protein is encoded by a sequence that shares at least 80%, at least 85%, at least 90%, at least 95%, at least 96%, at least 97%, at least 98%, at least 99%, or 100% identity to SEQ ID NO: 2. In some embodiments, the polynucleotide encoding the NGF protein is codon optimized. In some embodiments, the polynucleotide encoding the NGF protein comprises SEQ ID NO: 17. In some embodiments, the polynucleotide encoding the NGF protein comprises a sequence at least 80%, 85%, 90%, 95%, 96%, 97%, 98%, 99%, or 100% identical to SEQ ID NO: 17. In some embodiments, the polynucleotide encoding the NGF protein is encoded by a sequence that shares at least 80%, at least 85%, at least 90%, at least 95%, at least 96%, at least 97%, at least 98%, at least 99%, or 100% identity to SEQ ID NO: 17.

### Codon Optimized NGF

(SEQ ID NO: 17)
ATGAGCATGCTGTTTTACACCCTGATCACCGCCTTCCTGATCGGC
ATCCAGGCCGAGCCCCACAGCGAGAGCAACGTGCCCGCTGGACAC
ACAATCCCTCAGGCCCACTGGACAAAACTGCAGCATAGCCTGGAT
ACAGCCCTGAGAAGGGCCAGAAGCGCCCCTGCCGCCGCCATCGCC
GCTAGAGTGGCCGGCCAGACAAGAAATATCACCGTGGACCCCCGG
CTGTTCAAGAAAAGAAGACTGCGGTCTCCTAGAGTGCTCTTTTCA
ACACAGCCTCCTCGGGAAGCCGCTGATACCCAGGACCTGGACTTC
GAGGTGGGAGGCGCCGCTCCTTTCAATAGAACCCACAGATCCAAG
AGAAGCTCTAGCCACCCTATCTTCCACCGGGGCGAGTTCAGCGTG
TGCGACAGCGTTTCTGTGTGGGTGGGAGATAAGACCACCGCAACC
GATATCAAGGGCAAGGAAGTGATGGTGCTGGGCGAAGTGAACATC
AACAACAGCGTCTTTAAGCAGTACTTCTTCGAGACAAAGTGCCGG
GACCCAAACCCCGTGGACAGCGGCTGCAGAGGCATTGACTCCAAG
CACTGGAACTCCTACTGCACCACAACACACACCTTCGTGAAGGCC
CTGACCATGGACGGCAAACAAGCTGCCTGGCGGTTCATCAGAATC
GACACCGCCTGTGTCTGTGTGCTGAGCAGAAAGGCCGTGCGCCGG
GCCTAG

In some embodiments, the NGF protein is a human NGF protein.

In some embodiments, the neurotrophic factor is glial derived neurotrophic factor (GDNF) protein. As used herein, the term “GDNF protein” refers to a GDNF protein from any species. The term, “functional variant” refers to variants having sequence substitutions, insertions, deletions, and/or N- or C-terminal truncations, where the functional variant retains one or more functions of the reference protein, e.g., a native GDNF protein. GDNF is a 21 kDa protein that supports the growth, maintenance, and differentiation of a wide variety of neuronal systems by GDNF acting on its receptor GFRα-1 (Qi et al., Br J Ophthalmol. 92:1269-74 (2008)). In some embodiments, the GDNF protein comprises SEQ ID NO: 3. In some embodiments, the GDNF protein comprises a sequence at least 80%, 85%, 90%, 95%, 96%, 97%, 98%, 99%, or 100% identical to SEQ ID NO: 3. In some embodiments, the GDNF protein shares a sequence with at least 80%, at least 85%, at least 90%, at least 95%, at least 96%, at least 97%, at least 98%, at least 99%, or 100% identity to SEQ ID NO: 3. In some embodiments, the polynucleotide encoding the GDNF protein comprises SEQ ID NO: 4. In some embodiments, the polynucleotide encoding the GDNF protein comprises a sequence at least 80%, 85%, 90%, 95%, 96%, 97%, 98%, 99%, or 100% identical to SEQ ID NO: 4. In some embodiments, the GDNF protein is encoded by a sequence that shares a sequence at least 80%, at least 85%, at least 90%, at least 95%, at least 96%, at least 97%, at least 98%, at least 99%, or 100% identity to SEQ ID NO: 4. In some embodiments, the polynucleotide encoding the GDNF protein comprises SEQ ID NO: 18. In some embodiments, the polynucleotide encoding the GDNF protein comprises a sequence at least 80%, 85%, 90%, 95%, 96%, 97%, 98%, 99%, or 100% identical to SEQ ID NO: 18. In some embodiments, the polynucleotide encoding the GDNF protein is codon optimized. In some embodiments, the polynucleotide encoding the GDNF protein is encoded by a sequence that shares at least 80%, at least 85%, at least 90%, at least 95%, at least 96%, at least 97%, at least 98%, at least 99%, or 100% identity to SEQ ID NO: 18.

### Codon Optimized GDNF

(SEQ ID NO: 18)
ATGAACTGTGGGACGTTGTGGCCGTGTGCCTGGTCCTCCTGCACA
CCGCCTCCGCCTTTCCACTGCCTGCTGGAAAGCGGCCTCCTGAGG
CCCCTGCTGAAGATAGAAGCCTGGGCAGAAGAAGAGCCCCCTTCG
CCCTGAGCTCTGATAGCAACATGCCTGAGGACTACCCCGACCAGT
TCGACGACGTGATGGACTTTATCCAGGCCACCATCAAGAGACTGA
AACGGAGCCCTGACAAGCAGATGGCCGTGCTGCCTAGACGGGAAA
GAAATAGACAGGCCGCAGCTGCCAACCCCGAGAACAGCAGAGGCA
AAGGCCGGAGAGGCCAGAGGGGAAAAAACAGAGGATGTGTGCTGA
CCGCCATCCATCTGAACGTGACAGATCTGGGCCTGGGCTATGAGA
CCAAGGAAGAGCTGATCTTCAGATACTGCAGCGGCAGCTGCGACG
CCGCCGAGACAACCTACGACAAGATCCTGAAGAACCTGTCCCGGA
ATCGGCGGCTGGTGTCTGACAAGGTGGGCCAAGCTTGCTGCAGAC
CAATTGCCTTCGACGATGATCTGTCTTTCCTGGATGACAACCTGG
TGTACCACATCCTGAGAAAGCACAGCGCCAAGCGCTGCGGCTGTA
TCTAG

In some embodiments, the GDNF is human GDNF.

TABLE 1
Sequences of Neurotrophic Factors

| | Protein | Nucleotide (Open Reading Frame) |
|---|---|---|
| NGF | MSMLFYTLITAFLIGIQAEPHS | ATGTCCATGTTGTTCTACACTCTGATCA |
| | ESNVPAGHTIPQVHWTKLQHS | CAGCTTTTCTGATCGGCATACAGGCGG |
| | LDTALRRARSAPAAAIAARVA | AACCACACTCAGAGAGCAATGTCCCTG |
| | GQTRNITVDPRLFKKRRLRSPR | CAGGACACACCATCCCCCAAGTCCACT |
| | VLFSTQPPREAADTQDLDFEV | GGACTAAACTTCAGCATTCCCTTGACA |
| | GGAAPFNRTHRSKRSSSHPIFH | CTGCCCTTCGCAGAGCCCGCAGCGCCC |
| | RGEFSVCDSVSVWVGDKTTA | CGGCAGCGGCGATAGCTGCACGCGTGG |
| | TDIKGKEVMVLGEVNINNSVF | CGGGGCAGACCCGCAACATTACTGTGG |
| | KQYFFETKCRDPNPVDSGCRG | ACCCCAGGCTGTTTAAAAAGCGGCGAC |
| | IDSKHWNSY (SEQ ID NO: 1) | TCCGTTCACCCCGTGTGCTGTTTAGCAC |
| | | CCAGCCTCCCCGTGAAGCTGCAGACAC |
| | | TCAGGATCTGGACTTCGAGGTCGGTGG |
| | | TGCTGCCCCCTTCAACAGGACTCACAG |
| | | GAGCAAGCGGTCATCATCCCATCCCAT |
| | | CTTCCACAGGGGCGAATTCTCGGTGTG |
| | | TGACAGTGTCAGCGTGTGGGTTGGGGA |
| | | TAAGACCACCGCCACAGACATCAAGGG |
| | | CAAGGAGGTGATGGTGTTGGGAGAGGT |
| | | GAACATTAACAACAGTGTATTCAAACA |
| | | GTACTTTTTTGAGACCAAGTGCCGGGA |
| | | CCCAAATCCCGTTGACAGCGGGTGCCG |
| | | GGGCATTGACTCAAAGCACTGGAACTC |
| | | ATATTGTACCACGACTCACACCTTTGTC |
| | | AAGGCGCTGACCATGGATGGCAAGCA |
| | | GGCTGCCTGGCGGTTTATCCGGATAGA |
| | | TACGGCCTGTGTGTGTGTGCTCAGCAG |
| | | GAAGGCTGTGAGAAGAGCCTGA (SEQ |
| | | ID NO: 2) |
| GDNF | MKLWDVVAVCLVLLHTASAF | ATGAAGTTATGGGATGTCGTGGCTGTC |
| | PLPAGKRPPEAPAEDRSLGRR | TGCCTGGTGCTGCTCCACACCGCGTCC |
| | RAPFALSSDSNMPEDYPDQFD | GCCTTCCCGCTGCCCGCCGGTAAGAGG |
| | DVMDFIQATIKRLKRSPDKQM | CCTCCCGAGGCGCCCGCCGAAGACCGC |
| | AVLPRRERNRQAAAANPENSR | TCCCTCGGCCGCCGCCGCGCGCCCTTC |
| | GKGRRGQRGKNRGCVLTAIH | GCGCTGAGCAGTGACTCAAATATGCCA |
| | LNVTDLGLGYETKEELIFRYCS | GAGGATTATCCTGATCAGTTCGATGAT |
| | GSCDAAETTYDKILKNLSRNR | GTCATGGATTTTATTCAAGCCACCATTA |
| | RLVSDKVGQACCRPIAFDDDL | AAAGACTGAAAAGGTCACCAGATAAA |
| | SFLDDNL VYHILRKHSAKRCG | CAAATGGCAGTGCTTCCTAGAAGAGAG |
| | CI (SEQ ID NO: 3) | CGGAATCGGCAGGCTGCAGCTGCCAAC |
| | | CCAGAGAATTCCAGAGGAAAAGGTCG |
| | | GAGAGGCCAGAGGGGCAAAAACCGGG |
| | | GTTGTGTCTTAACTGCAATACATTTAAA |
| | | TGTCACTGACTTGGGTCTGGGCTATGA |
| | | AACCAAGGAGGAACTGATTTTTAGGTA |
| | | CTGCAGCGGCTCTTGCGATGCAGCTGA |
| | | GACAACGTACGACAAAATATTGAAAAA |
| | | CTTATCCAGAAATAGAAGGCTGGTGAG |
| | | TGACAAAGTAGGGCAGGCATGTTGCAG |
| | | ACCCATCGCCTTTGATGATGACCTGTC |
| | | GTTTTTAGATGATAACCTGGTTTACCAT |
| | | ATTCTAAGAAAGCATTCCGCTAAAAGG |
| | | TGTGGATGTATCTGA (SEQ ID NO: 4) |

In some embodiments, the expression cassette of the present disclosure comprises a promoter. The term “promoter” as used herein refers to a DNA sequence that directs the binding of RNA polymerase and thereby promotes RNA synthesis, i.e., a minimal sequence sufficient to direct transcription. A promoter can be operably linked to a transgene, e.g., a neurotrophic factor. Transcription of a transgene can be initiated and regulated by the promoter to which it is operably linked. For example, an expression cassette comprising a promoter operably linked to a transgene will express the transgene if the RNA synthesis initiated at the promoter. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

Any suitable promoter region or promoter sequence therein can be used in the subject polynucleotide cassettes, so long as the promoter region promotes expression of a polynucleotide sequence encoding an NGF or a GNDF protein in the at least one eye and/or lacrimal glands. In some embodiments, the promoter promotes expression of the gene in mammalian eye and/or lacrimal glands. In some embodiments, the expression cassette comprises a cell-type specific promoter. The promoter may specifically promote transcription in the cells of the eye and/or the cells of the lacrimal gland.

In some embodiments, the promoter is a CAG promoter. In some embodiments, the promoter comprises SEQ ID NO: 5. In some embodiments, the promoter comprises a sequence at least 95%, 96%, 97%, 98%, 99%, or 100% identical to SEQ ID NO: 5. In some embodiments, the promoter shares at least 90%, 95%, 96%, 97%, 98%, 99%, or 100% identity to a CAG promoter sequence (SEQ ID NO: 5).

### CAG Promoter

(SEQ ID NO: 5)
ataacttacggtaaatggcccgcctggctgaccgcccaacgaccc
ccgcccattgacgtcaataatgacgtatgttcccatagtaacgcc
aatagggactttccattgacgtcaatgggtggagtatttacggta
aactgcccacttggcagtacatcaagtgtatcatatgccaagtac
gccccctattgacgtcaatgacggtaaatggcccgcctggcatta
tgcccagtacatgaccttatgggactttcctacttggcagtacat
ctactcgaggccacgttctgcttcactctccccatctcccccccc
tccccacccccaattttgtatttatttattttttaattattttgt
gcagcgatgggggcggggggggggggggggcgcgcgccaggcggg
gggggggggcgaggggggggcggggcgaggcggagaggtgcggcg
gcagccaatcagagcggcgcgctccgaaagtttccttttatggcg
aggcggcggcggcggcggccctataaaaagcgaagcgcgcggggg
gggagcgggatcagccaccgcggtggcggcctagagtcgacgagg
aactgaaaaaccagaaagttaactg

In some embodiments, the promoter is a CMV promoter. In some embodiments, the promoter comprises SEQ ID NO: 16. In some embodiments, the promoter comprises a sequence at least 95%, 96%, 97%, 98%, 99%, or 100% identical to SEQ ID NO: 16. In some embodiments, the promoter shares at least 90%, 95%, 96%, 97%, 98%, 99%, or 100% identity to a CMV promoter sequence (SEQ ID NO: 16).

### CMV Promoter

(SEQ ID NO: 16)
ctagttattaatagtaatcaattacggggtcattagttcatagcc
catatatggagttccgcgttacataacttacggtaaatggcccgc
ctggctgaccgcccaacgacccccgcccattgacgtcaataatga
cgtatgttcccatagtaacgccaatagggactttccattgacgtc
aatgggtggactatttacggtaaactgcccacttggcagtacatc
aagtgtatcatatgccaagtacgccccctattgacgtcaatgacg
gtaaatggcccgcctggcattatgcccagtacatgaccttatggg
actttcctacttggcagtacatctacgtattagtcatcgctatta
ccatggtgatgcggttttggcagtacatcaatgggcgtggatagc
ggtttgactcacggggatttccaagtctccaccccattgacgtca
atgggagtttgttttggcaccaaaatcaacgggactttccaaaat
gtcgtaacaactccgccccattgacgcaaatgggcggtaggcgtg
tacggtgggaggtctatataagcagagctctctggctaactagag
aacccactgcttactggcttatcgaaat

In some embodiments, the expression cassette comprises a nucleotide sequence operably linked to a polyadenylation sequence. Suitable polyadenylation sequences include bovine growth hormone polyA signal (bGHpolyA) and short poly A signal. In some embodiments, the polyadenylation sequence comprises SEQ ID NO: 21. Optionally the rAAV vectors of the disclosure comprise the Woodchuck Post-transcriptional Regulatory Element (WPRE). In some embodiments, the rAAV vector comprises a WPRE comprising SEQ ID NO: 22.

>bGHpolyA Sequence
(SEQ ID NO: 21)
cgactgtgccttctagttgccagccatctgttgtttgcccctccc
ccgtgccttccttgaccctggaaggtgccactcccactgtccttt
cctaataaaatgaggaaattgcatcgcattgtctgagtaggtgtc
attctattctggggggtggggggggcaggacagcaagggggagga
ttgggaagacaatagcaggcatgctggggatgcggtgggctctat
gg

### WPRE Sequence

(SEQ ID NO: 22)
tcctgttaatcaacctctggattacaaaatttgtgaaagattgac
tgatattcttaactatgttgctccttttacgctgtgtggatatgc
tgctttaatgcctctgtatcatgctattgcttcccgtacggcttt
cgttttctcctccttgtataaatcctggttgctgtctctttatga
ggagttgtggcccgttgtccgtcaacgtggcgtggtgtgctctgt
gtttgctgacgcaacccccactggctggggcattgccaccacctg
tcaactcctttctgggactttcgctttccccctccctatcgccac
ggcagaactcatcgccgcctgccttgcccgctgctggacaggggc
taggttgctgggcactgataattccgtggtgttgtcggggaagct
gacgtc

In some embodiments, the expression cassette comprises SEQ ID NO: 23. In some embodiments, the expression cassette comprises a sequence at least 90%, 95%, 96%, 97%, 98%, 99%, or 100% identical to SEQ ID NO: 23. In some embodiments, the expression cassette comprises a sequence sharing at least 90%, 95%, 96%, 97%, 98%, 99%, or 100% identity to SEQ ID NO: 23.

### Expression Cassette Comprising Sequence Encoding GDNF

(SEQ ID NO: 23)
gcgcgctcgctcgctcactgaggccgcccgggcaaagcccgggcg
tcgggcgacctttggtcgcccggcctcagtgagcgagcgagcgcg
cagagagggagtggccaactccatcactaggggttccttgtagtt
aatgattaacggatctttacaaattcaagccaggtgatttcaaca
aattttgctgacgatttaggcgcactatcccctaaactacaaatt
agaaaatagcgttccttgacactagtctagttattaatagtaatc
aattacggggtcattagttcatagcccatatatggagttccgcgt
tacataacttacggtaaatggcccgcctggctgaccgcccaacga
cccccgcccattgacgtcaataatgacgtatgttcccatagtaac
gccaatagggactttccattgacgtcaatgggtggactatttacg
gtaaactgcccacttggcagtacatcaagtgtatcatatgccaag
tacgccccctattgacgtcaatgacggtaaatggcccgcctggca
ttatgcccagtacatgaccttatgggactttcctacttggcagta
catctacgtattagtcatcgctattaccatggtgatgcggttttg
gcagtacatcaatgggcgtggatagcggtttgactcacggggatt
tccaagtctccaccccattgacgtcaatgggagtttgttttggca
ccaaaatcaacgggactttccaaaatgtcgtaacaactccgcccc
attgacgcaaatgggcggtaggcgtgtacggtgggaggtctatat
aagcagagctctctggctaactagagaacccactgcttactggct
tatcgaaataagctttctcaggggagatctcgtttagtgaaccgt
cagatcctcactctcttccgcatcgctgtctgcgagggccagctg
ttgggctcgcggttgaggacaaactcttcgcggtctttccagtac
tcttggatcggaaacccgtcggcctccgaacggtactccgccacc
gagggacctgagcgagtccgcatcgaccggatcggaaaacctctc
gagaaaggcgtctaaccagtcacagtcgcaaggtaggctgagcac
cgtggcgggcggcagcgggtggcggtcggggttgtttctggcgga
ggtgctgctgatgatgtaattaaagtaggcggtcttgagacggcg
gatggtcgaggtgaggtgtggcaggcttgagatccagctgttggg
gtgagtactccctctcaaaagcgggcattacttctgcgctaagat
tgtcagtttccaaaaacgaggaggatttgatattcacctggcccg
atctggccatacacttgagtgacaatgacatccactttgcctttc
tctccacaggtgtccactcccaggtccaagtttaaacgccgccgc
catgaagctgtgggacgttgtggccgtgtgcctggtcctcctgca
caccgcctccgcctttccactgcctgctggaaagcggcctcctga
ggcccctgctgaagatagaagcctgggcagaagaagagccccctt
cgccctgagctctgatagcaacatgcctgaggactaccccgacca
gttcgacgacgtgatggactttatccaggccaccatcaagagact
gaaacggagccctgacaagcagatggccgtgctgcctagacggga
aagaaatagacaggccgcagctgccaaccccgagaacagcagagg
caaaggccggagaggccagaggggaaaaaacagaggatgtgtgct
gaccgccatccatctgaacgtgacagatctgggcctgggctatga
gaccaaggaagagctgatcttcagatactgcagcggcagctgcga
cgccgccgagacaacctacgacaagatcctgaagaacctgtcccg
gaatcggcggctggtgtctgacaaggtgggccaagcttgctgcag
accaattgccttcgacgatgatctgtctttcctggatgacaacct
ggtgtaccacatcctgagaaagcacagcgccaagcgctgcggctg
tatctagtcctgttaatcaacctctggattacaaaatttgtgaaa
gattgactgatattcttaactatgttgctccttttacgctgtgtg
gatatgctgctttaatgcctctgtatcatgctattgcttcccgta
cggctttcgttttctcctccttgtataaatcctggttgctgtctc
tttatgaggagttgtggcccgttgtccgtcaacgtggcgtggtgt
gctctgtgtttgctgacgcaacccccactggctggggcattgcca
ccacctgtcaactcctttctgggactttcgctttccccctcccta
tcgccacggcagaactcatcgccgcctgccttgcccgctgctgga
caggggctaggttgctgggcactgataattccgtggtgttgtcgg
ggaagctgacgtccgactgtgccttctagttgccagccatctgtt
gtttgcccctcccccgtgccttccttgaccctggaaggtgccact
cccactgtcctttcctaataaaatgaggaaattgcatcgcattgt
ctgagtaggtgtcattctattctggggggggggtggggcaggaca
gcaagggggaggattgggaagacaatagcaggcatgctggggatg
cggtgggctctatggagcgctggctagaattacctaccggcctcc
accataccttcgatattcgcgcccactctcccattaatccgcaca
agtggatgtgatgcgattgcccgctaagatagttaatcattaact
acaaggaacccctagtgatggagttggccactccctctctgcgcg
ctcgctcgctcactgaggccgggcgaccaaaggtcgcccgacgcc
cgggctttgcccgggcggcctcagtgagcgagcgagcgcgc

In some embodiments, the expression cassette comprises SEQ ID NO: 24. In some embodiments, the expression cassette comprises a sequence at least 90%, 95%, 96%, 97%, 98%, 99%, or 100% identical to SEQ ID NO: 24. In some embodiments, the expression cassette comprises a sequence shares at least 90%, 95%, 96%, 97%, 98%, 99%, or 100% identity to SEQ ID NO: 24.

### Expression Cassette Comprising Sequence Encoding NGF

(SEQ ID NO: 24)
gcgcgctcgctcgctcactgaggccgcccgggcaaagcccgggcg
tcgggcgacctttggtcgcccggcctcagtgagcgagcgagcgcg
cagagagggagtggccaactccatcactaggggttccttgtagtt
aatgattaacggatctttacaaattcaagccaggtgatttcaaca
aattttgctgacgatttaggcgcactatcccctaaactacaaatt
agaaaatagcgttccttgacactagtctagttattaatagtaatc
aattacggggtcattagttcatagcccatatatggagttccgcgt
tacataacttacggtaaatggcccgcctggctgaccgcccaacga
cccccgcccattgacgtcaataatgacgtatgttcccatagtaac
gccaatagggactttccattgacgtcaatgggtggactatttacg
gtaaactgcccacttggcagtacatcaagtgtatcatatgccaag
tacgccccctattgacgtcaatgacggtaaatggcccgcctggca
ttatgcccagtacatgaccttatgggactttcctacttggcagta
catctacgtattagtcatcgctattaccatggtgatgcggttttg
gcagtacatcaatgggcgtggatagcggtttgactcacggggatt
tccaagtctccaccccattgacgtcaatgggagtttgttttggca
ccaaaatcaacgggactttccaaaatgtcgtaacaactccgcccc
attgacgcaaatgggcggtaggcgtgtacggtgggaggtctatat
aagcagagctctctggctaactagagaacccactgcttactggct
tatcgaaataagctttctcaggggagatctcgtttagtgaaccgt
cagatcctcactctcttccgcatcgctgtctgcgagggccagctg
ttgggctcgcggttgaggacaaactcttcgcggtctttccagtac
tcttggatcggaaacccgtcggcctccgaacggtactccgccacc
gagggacctgagcgagtccgcatcgaccggatcggaaaacctctc
gagaaaggcgtctaaccagtcacagtcgcaaggtaggctgagcac
cgtggcgggcggcagcgggggcggtcggggttgtttctggcggag
gtgctgctgatgatgtaattaaagtaggcggtcttgagacggcgg
atggtcgaggtgaggtgtggcaggcttgagatccagctgttgggg
tgagtactccctctcaaaagcgggcattacttctgcgctaagatt
gtcagtttccaaaaacgaggaggatttgatattcacctggcccga
tctggccatacacttgagtgacaatgacatccactttgcctttct
ctccacaggtgtccactcccaggtccaagtttaaacgccgccgcc
atgagcatgctgttttacaccctgatcaccgccttcctgatcggc
atccaggccgagccccacagcgagagcaacgtgcccgctggacac
acaatccctcaggcccactggacaaaactgcagcatagcctggat
acagccctgagaagggccagaagcgcccctgccgccgccatcgcc
gctagagtggccggccagacaagaaatatcaccgtggacccccgg
ctgttcaagaaaagaagactgcggtctcctagagtgctcttttca
acacagcctcctcgggaagccgctgatacccaggacctggacttc
gaggtgggaggcgccgctcctttcaatagaacccacagatccaag
agaagctctagccaccctatcttccaccggggcgagttcagcgtg
tgcgacagcgtttctgtgtggggggagataagaccaccgcaaccg
atatcaagggcaaggaagtgatggtgctgggcgaagtgaacatca
acaacagcgtctttaagcagtacttcttcgagacaaagtgccggg
acccaaaccccgtggacagcggctgcagaggcattgactccaagc
actggaactcctactgcaccacaacacacaccttcgtgaaggccc
tgaccatggacggcaaacaagctgcctggcggttcatcagaatcg
acaccgcctgtgtctgtgtgctgagcagaaaggccgtgcgccggg
cctagtcctgttaatcaacctctggattacaaaatttgtgaaaga
ttgactgatattcttaactatgttgctccttttacgctgtgtgga
tatgctgctttaatgcctctgtatcatgctattgcttcccgtacg
gctttcgttttctcctccttgtataaatcctggttgctgtctctt
tatgaggagttgtggcccgttgtccgtcaacgtggcgtggtgtgc
tctgtgtttgctgacgcaacccccactggctggggcattgccacc
acctgtcaactcctttctgggactttcgctttccccctccctatc
gccacggcagaactcatcgccgcctgccttgcccgctgctggaca
ggggctaggttgctgggcactgataattccgtggtgttgtcgggg
aagctgacgtccgactgtgccttctagttgccagccatctgttgt
ttgcccctcccccgtgccttccttgaccctggaaggtgccactcc
cactgtcctttcctaataaaatgaggaaattgcatcgcattgtct
gagtaggtgtcattctattctggggggtggggggggcaggacagc
aagggggaggattgggaagacaatagcaggcatgctggggatgcg
gtgggctctatggagcgctggctagaattacctaccggcctccac
cataccttcgatattcgcgcccactctcccattaatccgcacaag
tggatgtgatgcgattgcccgctaagatagttaatcattaactac
aaggaacccctagtgatggagttggccactccctctctgcgcgct
cgctcgctcactgaggccgggcgaccaaaggtcgcccgacgcccg
ggctttgcccgggcggcctcagtgagcgagcgagcgcgc

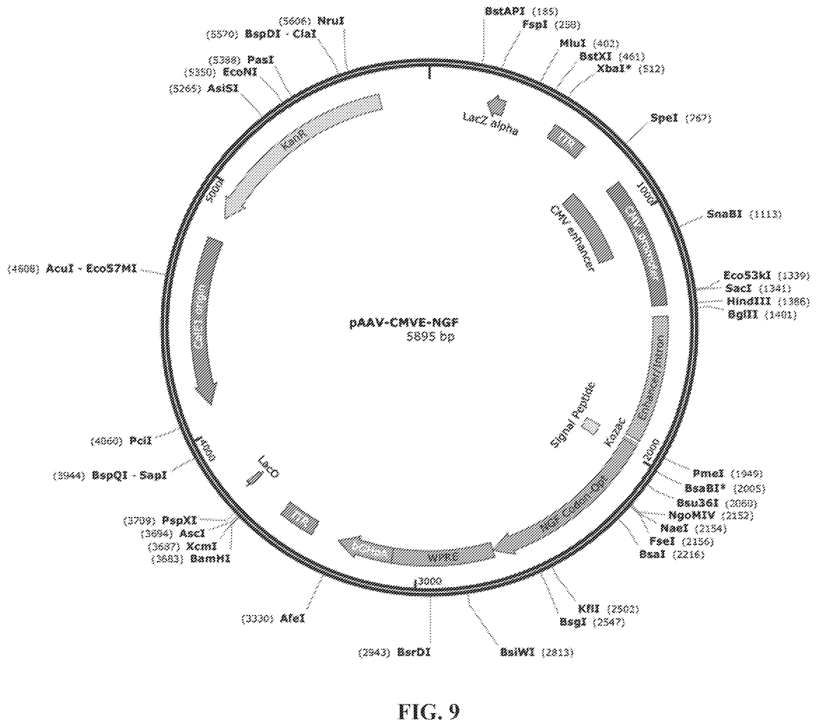

In some embodiments, the rAAV virion comprises the nucleotide sequence of SEQ ID NO: 25. In some embodiments, the rAAV virion comprises the expression cassette depicted by the plasmid in FIG. 9. In some embodiments, the expression cassette comprises a sequence at least 90%, 95%. 96%, 97%, 98%. 99%, or 100% identical to SEQ ID NO: 25.

TABLE 2
pAAV-CMVE-NGF Sequence

| | |
|---|---|
| pAAV-CMVE-NGF | tcgcgcgtttcggtgatgacggtga |
| comprising a: | aaacctctgacacatgcagctcccg |
| 5' ITR | gagacggtcacagcttgtctgtaag |
| CMV Promoter | cggatgccgggagcagacaagcccg |
| NGF Codon- | tcagggcgcgtcagcgggtgttggc |
| optimized | gggtgtcggggctggcttaactatg |
| sequence | cggcatcagagcagattgtactgag |
| WPRE3' ITR | agtgcaccatatgcggtgtgaaata |
| Where each | ccgcacagatgcgtaaggagaaaat |
| domain above | accgcatcaggcgccattcgccatt |
| corresponds | caggctgcgcaactgttgggaaggg |
| in the sequence | cgatcggtgcgggcctcttcgctat |
| to a region in | tacgccagctggcgaaagggggatg |
| the same | tgctgcaaggcgattaagttgggta |
| typeface. | acgccagggttttcccagtcacgac |
| (SEQ ID NO: 25) | gttgtaaaacgacggccagtgaatt |
| | gacgcgtattgggattgacctaacg |
| | aaaagtgaacttcataatacgtgct |
| | gtcccacgcacatggtagatttgga |
| | caaaattgaatggagtctgatcaac |
| | cttcacaccgatctagatatcgcgc |
| | gctcgctcgctcactgaggccgccc |
| | gggcaaagcccgggcgtcgggcgac |
| | ctttggtcgcccggcctcagtgagc |
| | gagcgagcgcgcagagagggagtgg |
| | ccaactccatcactaggggttcctt |
| | gtagttaatgattaacggatcttta |
| | caaattcaagccaggtgatttcaac |
| | aaattttgctgacgatttaggcgca |
| | ctatcccctaaactacaaattagaa |
| | aatagcgttccttgacactagtcta |
| | gttattaatagtaatcaattacggg |
| | gtcattagttcatagcccatatatg |
| | gagttccgcgttacataacttacgg |
| | taaatggcccgcctggctgaccgcc |
| | caacgacccccgcccattgacgtca |
| | ataatgacgtatgttcccatagtaa |
| | cgccaatagggactttccattgacg |
| | tcaatgggtggactatttacggtaa |
| | actgcccacttggcagtacatcaag |
| | tgtatcatatgccaagtacgccccc |
| | tattgacgtcaatgacggtaaatgg |
| | cccgcctggcattatgcccagtaca |
| | tgaccttatgggactttcctacttg |
| | gcagtacatctacgtattagtcatc |
| | gctattaccatggtgatgcggtttt |
| | ggcagtacatcaatgggcgtggata |
| | gcggtttgactcacggggatttcca |
| | agtctccaccccattgacgtcaatg |
| | ggagtttgttttggcaccaaaatca |
| | acgggactttccaaaatgtcgtaac |
| | aactccgccccattgacgcaaatgg |
| | gcggtaggcgtgtacggtgggaggt |
| | ctatataagcagagctctctggcta |
| | actagagaacccactgcttactggc |
| | ttatcgaaataagctttctcagggg |
| | agatctcgtttagtgaaccgtcaga |
| | tcctcactctcttccgcatcgctgt |
| | ctgcgagggccagctgttgggctcg |
| | cggttgaggacaaactcttcgcggt |
| | ctttccagtactcttggatcggaaa |
| | cccgtcggcctccgaacggtactcc |
| | gccaccgagggacctgagcgagtcc |
| | gcatcgaccggatcggaaaacctct |
| | cgagaaaggcgtctaaccagtcaca |
| | gtcgcaaggtaggctgagcaccgtg |
| | gcgggcggcagcgggtggcggtcgg |
| | ggttgtttctggcggaggtgctgct |
| | gatgatgtaattaaagtaggcggtc |
| | ttgagacggcggatggtcgaggtga |
| | ggtgtggcaggcttgagatccagct |
| | gttggggtgagtactccctctcaaa |
| | agcgggcattacttctgcgctaaga |
| | ttgtcagtttccaaaaacgaggagg |
| | atttgatattcacctggcccgatct |
| | ggccatacacttgagtgacaatgac |
| | atccactttgcctttctctccacag |
| | gtgtccactcccaggtccaagttta |
| | aacgccgccgccatgagcatgctgt |
| | tttacaccctgatcaccgccttcct |
| | gatcggcatccaggccgagccccac |
| | agcgagagcaacgtgcccgctggac |
| | acacaatccctcaggcccactggac |
| | aaaactgcagcatagcctggataca |
| | gccctgagaagggccagaagcgccc |
| | ctgccgccgccatcgccgctagagt |
| | ggccggccagacaagaaatatcacc |
| | gtggacccccggctgttcaagaaaa |
| | gaagactgcggtctcctagagtgct |
| | cttttcaacacagcctcctcgggaa |
| | gccgctgatacccaggacctggact |
| | tcgaggtgggaggcgccgctccttt |
| | caatagaacccacagatccaagaga |
| | agctctagccaccctatcttccacc |
| | ggggcgagttcagcgtgtgcgacag |
| | cgtttctgtgtggggggagataaga |
| | ccaccgcaaccgatatcaagggcaa |
| | ggaagtgatggtgctgggcgaagtg |
| | aacatcaacaacagcgtctttaagc |
| | agtacttcttcgagacaaagtgccg |
| | ggacccaaaccccgtggacagcggc |
| | tgcagaggcattgactccaagcact |
| | ggaactcctactgcaccacaacaca |
| | caccttcgtgaaggccctgaccatg |
| | gacggcaaacaagctgcctggcggt |
| | tcatcagaatcgacaccgcctgtgt |
| | ctgtgtgctgagcagaaaggccgtg |
| | cgccgggcctagtcctgttaatcaa |
| | cctctggattacaaaatttgtgaaa |
| | gattgactgatattcttaactatgt |
| | tgctccttttacgctgtgtggatat |
| | gctgctttaatgcctctgtatcatg |
| | ctattgcttcccgtacggctttcgt |
| | tttctcctccttgtataaatcctgg |
| | ttgctgtctctttatgaggagttgt |
| | ggcccgttgtccgtcaacgtggcgt |
| | ggtgtgctctgtgtttgctgacgca |
| | acccccactggctggggcattgcca |
| | ccacctgtcaactcctttctgggac |
| | tttcgctttccccctccctatcgcc |
| | acggcagaactcatcgccgcctgcc |
| | ttgcccgctgctggacaggggctag |
| | gttgctgggcactgataattccgtg |
| | gtgttgtcggggaagctgacgtccg |
| | actttcgctttccccctccctatcg |
| | ccacggcagaactcatcgccgcctg |
| | ccttgcccgctgctggacaggggct |
| | aggttgctgggcactgataattccg |
| | tggtgttgtcggggaagctgacgtc |
| | cgactgtgccttctagttgccagcc |
| | atctgttgtttgcccctcccccgtg |
| | ccttccttgaccctggaaggtgcca |
| | ctcccactgtcctttcctaataaaa |
| | tgaggaaattgcatcgcattgtctg |
| | agtaggtgtcattctattctggggg |
| | gtggggggggcaggacagcaagggg |
| | gaggattgggaagacaatagcaggc |
| | atgctggggatgcggtgggctctat |
| | ggagcgctggctagaattacctacc |
| | ggcctccaccataccttcgatattc |
| | gcgcccactctcccattaatccgca |
| | caagtggatgtgatgcgattgcccg |
| | ctaagatagttaatcattaactaca |
| | aggaacccctagtgatggagttggc |
| | cactccctctctgcgcgctcgctcg |
| | ctcactgaggccgggcgaccaaagg |
| | tcgcccgacgcccgggctttgcccg |
| | ggcggcctcagtgagcgagcgagcg |
| | cgcgatatcgaatcgaatgcagtga |
| | tctcccaggtgcgaatcaaaaatac |
| | taggtaactagaggatttgcgacgt |
| | tctaagcgttggtccatgtgaatcg |
| | ccatccaggatcccaatggcgcgcc |
| | gagcttggctcgagcatggtcatag |
| | ctgtttcctgtgtgaaattgttatc |
| | cgctcacaattccacacaacatacg |
| | agccggaagcataaagtgtaaagcc |
| | tggggtgcctaatgagtgagctaac |
| | tcacattaattgcgttgcgctcact |
| | gcccgctttccagtcgggaaacctg |
| | tcgtgccagctgcattaatgaatcg |
| | gccaacgcgcggggagaggcggttt |
| | gcgtattgggcgctcttccgcttcc |
| | tcgctcactgactcgctgcgctcgg |
| | tcgttcggctgcggcgagcggtatc |
| | agctcactcaaaggcggtaatacgg |
| | ttatccacagaatcaggggataacg |
| | caggaaagaacatgtgagcaaaagg |
| | ccagcaaaaggccaggaaccgtaaa |
| | aaggccgcgttgctggcgtttttcc |
| | ataggctccgcccccctgacgagca |
| | tcacaaaaatcgacgctcaagtcag |
| | aggtggcgaaacccgacaggactat |
| | aaagataccaggcgtttccccctgg |
| | aagctccctcgtgcgctctcctgtt |
| | ccgaccctgccgcttaccggatacc |
| | tgtccgcctttctcccttcgggaag |
| | cgtggcgctttctcatagctcacgc |
| | tgtaggtatctcagttcggtgtagg |
| | tcgttcgctccaagctgggctgtgt |
| | gcacgaaccccccgttcagcccgac |
| | cgctgcgccttatccggtaactatc |
| | gtcttgagtccaacccggtaagaca |
| | cgacttatcgccactggcagcagcc |
| | actggtaacaggattagcagagcga |
| | ggtatgtaggcggtgctacagagtt |
| | cttgaagtggtggcctaactacggc |
| | tacactagaagaacagtatttggta |
| | tctgcgctctgctgaagccagttac |
| | cttcggaaaaagagttggtagctct |
| | tgatccggcaaacaaaccaccgctg |
| | gtagcggtggtttttttgtttgcaa |
| | gcagcagattacgcgcagaaaaaaa |
| | ggatctcaagaagatcctttgatct |
| | tttctacggggtctgacgctcagtg |
| | gaacgaaaactcacgttaagggatt |
| | ttggtcatgagattatcaaaaagga |
| | tcttcacctagatccttttaaatta |
| | aaaatgaagttttaaatcaatctaa |
| | agtatatatgagtaaacttggtctg |
| | acagttagaaaaactcatcgagcat |
| | caaatgaaactgcaatttattcata |
| | tcaggattatcaataccatattttt |
| | gaaaaagccgtttctgtaatgaagg |
| | agaaaactcaccgaggcagttccat |
| | aggatggcaagatcctggtatcggt |
| | ctgcgattccgactcgtccaacatc |
| | aatacaacctattaatttcccctcg |
| | tcaaaaataaggttatcaagtgaga |
| | aatcaccatgagtgacgactgaatc |
| | cggtgagaatggcaaaagtttatgc |
| | atttctttccagacttgttcaacag |
| | gccagccattacgctcgtcatcaaa |
| | atcactcgcatcaaccaaaccgtta |
| | ttcattcgtgattgcgcctgagcga |
| | gacgaaatacgcgatcgctgttaaa |
| | aggacaattacaaacaggaatcgaa |
| | tgcaaccggcgcaggaacactgcca |
| | gcgcatcaacaatattttcacctga |
| | atcaggatattcttctaatacctgg |
| | aatgctgttttcccagggatcgcag |
| | tggtgagtaaccatgcatcatcagg |
| | agtacggataaaatgcttgatggtc |
| | ggaagaggcataaattccgtcagcc |
| | agtttagtctgaccatctcatctgt |
| | aacatcattggcaacgctacctttg |
| | ccatgtttcagaaacaactctggcg |
| | catcgggcttcccatacaatcgata |
| | gattgtcgcacctgattgcccgaca |
| | ttatcgcgagcccatttatacccat |
| | ataaatcagcatccatgttggaatt |
| | taatcgcggcctagagcaagacgtt |
| | tcccgttgaatatggctcatactct |
| | tcctttttcaatattattgaagcat |
| | ttatcagggttattgtctcatgagc |
| | ggatacatatttgaatgtatttaga |
| | aaaataaacaaataggggttccgcg |
| | cacatttccccgaaaagtgccacct |
| | gacgtctaagaaaccattattatca |
| | tgacattaacctataaaaataggcg |
| | tatcacgaggccctttcgtc |

### Recombinant AAV Virion

In some embodiments of the present disclosure, the subject expression cassettes are used to deliver a neurotrophic factor to at least one eye and/or lacrimal gland of a subject, e.g., to treat an ocular disorder. Accordingly, in some embodiments, of the disclosure, the composition that provides for the expression of a neurotrophic factor in at least one eye and/or lacrimal gland of a subject is a gene delivery vector, wherein the gene delivery vector comprises the polynucleotide cassettes of the present disclosure.

In some embodiments, the gene delivery vector is an rAAV virion. In such embodiments, the subject expression cassette comprises AAV inverted terminal repeat sequences. In some embodiments, the expression cassette is flanked on the 5′ and 3′ ends by functional AAV inverted terminal repeat (ITR) sequences. By “functional AAV ITR sequences” is meant that the ITR sequences function as intended for the rescue, replication and packaging of the AAV virion. Hence, AAV ITRs for use in the gene delivery vectors of the present disclosure need not have a wild-type nucleotide sequence, and may be altered by the insertion, deletion or substitution of nucleotides or the AAV ITRs may be derived from any of several AAV serotypes, e.g., AAV1, AAV2, AAV3, AAV4, AAV5, AAV6, AAV7, AAV8, AAV9, AAV10. In some embodiments, the AAV ITR is derived from AAV1. In some embodiments, the AAV ITR is derived from AAV2. In some embodiments, the AAV ITR is derived from AAV3. In some embodiments, the AAV ITR is derived from AAV4. In some embodiments, the AAV ITR is derived from AAV5. In some embodiments, the AAV ITR is derived from AAV6. In some embodiments, the AAV ITR is derived from AAV7. In some embodiments, the AAV ITR is derived from AAV9. In some embodiments, the AAV ITR is derived from AAV1. In some embodiments, the AAV ITR is derived from AAV10. Certain rAAV virions have the wild type REP and CAP genes deleted in whole or part, but retain functional flanking ITR sequences. In some embodiments, the 5′ ITR comprises SEQ ID NO: 19. In some embodiments, the 5′ ITR comprises a sequence at least 90%, 95%, 96%, 97%, 98%, 99%, or 100% identical to SEQ ID NO: 19. In some embodiments, the 5′ ITR shares a sequence with at least 90%, 95%, 96%, 97%, 98%, 99%, or 100% identity to SEQ ID NO: 19.

### Illustrative 5′ ITR Sequence

(SEQ ID NO: 19)
gcgcgctcgctcgctcactgaggccgcccgggcaaagcccgggcg
tcgggcgacctttggtcgcccggcctcagtgagcgagcgagcgcg
cagagagggagtggccaactccatcactaggggttccttgtagtt
aatgattaac

In some embodiments, the 5′ ITR comprises SEQ ID NO: 20. In some embodiments, the 5′ ITR comprises a sequence at least 90%, 95%, 96%, 97%, 98%, 99%, or 100% identical to SEQ ID NO: 20. In some embodiments, the 5′ ITR shares a sequence with at least 90%, 95%, 96%, 97%, 98%, 99%, or 100% identity to SEQ ID NO: 20.

Illustrative 3′ ITR Sequence

(SEQ ID NO: 20)
gttaatcattaactacaaggaacccctagtgatggagttggccac
tccctctctgcgcgctcgctcgctcactgaggccgggcgaccaaa
ggtcgcccgacgcccgggctttgcccgggcggcctcagtgagcga
gcgagcgcgc

In such embodiments, the rAAV virion comprises an AAV capsid, derived from any adeno-associated virus serotype known in the art, or prospectively discovered, including without limitation, AAV1, AAV2, AAV3, AAV4, AAV5, AAV6, AAV7, AAV8, AAV9, AAV10, etc. For example, the AAV capsid may be a wild type (or “native”) capsid. In some embodiments, the rAAV virion comprises an AAV capsid derived from AAV1. In some embodiments, the rAAV virion comprises an AAV capsid derived from AAV2. In some embodiments, the rAAV virion comprises an AAV capsid derived from AAV3. In some embodiments, the rAAV virion comprises an AAV capsid derived from AAV4. In some embodiments, the rAAV virion comprises an AAV capsid derived from AAV5. In some embodiments, the rAAV virion comprises an AAV capsid derived from AAV6. In some embodiments, the rAAV virion comprises an AAV capsid derived from AAV7. In some embodiments, the rAAV virion comprises an AAV capsid derived from AAV8. In some embodiments, the rAAV virion comprises an AAV capsid derived from AAV9. In some embodiments, the rAAV virion comprises an AAV capsid derived from AAV10. AAV capsids of particular interest include AAV2, AAV5, AAV8, and AAV9 (Table 3).

However, as with the ITRs, the capsid need not have a wild-type nucleotide sequence, but rather may be altered by the insertion, deletion or substitution of nucleotides in the VP1, VP2 or VP3 sequence, so long as the capsid is able to transduce cells of the eye and/or lacrimal glands. Put another way, the AAV capsid may be a variant AAV capsid. In some embodiments, the rAAV virion is a “pseudotyped” AAV created by using the capsid (cap) gene of one AAV and the rep gene and ITRs from a different AAV, e.g., a pseudotyped AAV2 created by using rep from AAV2 and cap from AAV1, AAV3, AAV4, AAV5, AAV6, AAV7, AAV8, or AAV9 together with a plasmid containing a vector based on AAV2. For example, the rAAV virion may be rAAV2/1, rAAV2/3, rAAV2/4, rAAV2/5, rAAV2/6, rAAV2/7, rAAV2/8, rAAV2/9, etc. In some embodiments, the rAAV is rAAV2/1. In some embodiments, the rAAV is rAAV2/3. In some embodiments, the rAAV is rAAV2/4. In some embodiments, the rAAV is rAAV2/5. In some embodiments, the rAAV is rAAV2/6. In some embodiments, the rAAV is rAAV2/7. In some embodiments, the rAAV is rAAV2/8. In some embodiments, the rAAV is rAAV2/9.

In some embodiments, the rAAV is replication defective, in that the rAAV virion cannot independently further replicate and package its genome. For example, when eye and/or lacrimal glands are transduced with rAAV virions, the gene is expressed in the transduced eye and/or lacrimal gland, however, due to the fact that the transduced eye and/or lacrimal glands lack AAV rep and cap genes and accessory function genes, the rAAV is not able to replicate.

rAAV virions of the present disclosure encapsulating the expression cassettes as described herein, can be produced using helper-free production. rAAVs are replication-deficient viruses and normally require components from a live helper virus, such as adenovirus, in a host cell for packaging of infectious rAAV virions. rAAV helper-free production systems allow the production of infectious rAAV virions without the use of a live helper virus. In the helper-free system, a host packaging cell line is co-transfected with three plasmids. A first plasmid contains adenovirus gene products (i.e. E2A, E4, and VA RNA genes) needed for the packaging of rAAV virions. A second plasmid contains the required AAV genes (i.e., REP and CAP genes). A third plasmid contains the polynucleotide sequence encoding the protein of interest and a promoter flanked by ITRs. A host packaging cell line can be, for example, AAV-293 host cells. Suitable host cells contain additional components required for packaging infectious rAAV virions that are not supplied by the plasmids. In some embodiments, the CAP genes can encode, for example, AAV capsid proteins as described herein. In some embodiments, the promoter is a promoter sequence as described herein. In some embodiments, the promoter sequence is a CAG sequence. In some embodiments, the protein of interest is a neurotrophic factor. In some embodiments, the neurotrophic factor is NGF. In some embodiments, the neurotrophic factor is GDNF.

AAV serotypes shown to infect the eye and/or lacrimal gland include AAV2, AAV5, AAV 5w8, and AAV9 (Rocha et al., supra). In some embodiments, the AAV serotype used to infect the eye and/or lacrimal gland is AAV2. In some embodiments, the AAV capsid protein comprises SEQ ID NO: 6. In some embodiments, the AAV capsid protein comprises a sequence at least 95%, 96%, 97%, 98%, 99%, or 100% identical to SEQ ID NO: 6. In some embodiments, the AAV capsid protein shares at least 95%, 98%, or 100% identity to the AAV2 VP1 protein (SEQ ID NO: 6). In some embodiments, the polynucleotide sequence encoding the AAV2 VP1 protein comprises SEQ ID NO: 7. In some embodiments, the polynucleotide sequence encoding the AAV2 VP 1 protein comprises a sequence at least 95%, 96%, 97%, 98%, 99%, or 100% identical to SEQ ID NO: 7. In some embodiments, the polynucleotide sequence encoding the AAV2 VP1 protein shares at least 95%, 98%, or 100% identity with SEQ ID NO: 7. In some embodiments, the AAV capsid protein comprises SEQ ID NO: 8. In some embodiments, the AAV capsid protein comprises a sequence at least 95%, 96%, 97%, 98%, 99%, or 100% identical to SEQ ID NO: 8. In some embodiments, the AAV capsid protein shares at least 95%, 98%, or 100% identity to the AAV2 VP3 protein (SEQ ID NO: 8). In some embodiments, the polynucleotide sequence encoding the AAV2 VP3 protein comprises SEQ ID NO: 9. In some embodiments, the polynucleotide sequence encoding the AAV capsid protein comprises a sequence at least 95%, 96%, 97%, 98%, 99%, or 100% identical to SEQ ID NO: 9. In some embodiments, the polynucleotide sequence encoding the AAV2 VP3 protein shares at least 95%, 98%, or 100% identity with SEQ ID NO: 9. In some embodiments, the AAV serotype used to infect the eye and/or lacrimal gland is AAV5. In some embodiments, the AAV capsid protein comprises SEQ ID NO: 10. In some embodiments, the AAV capsid protein comprises a sequence at least 95%, 96%, 97%, 98%, 99%, or 100% identical to SEQ ID NO: 10. In some embodiments, the AAV capsid protein shares at least 95%, 98%, or 100% identity to the AAV5 capsid protein (SEQ ID NO: 10). In some embodiments, the polynucleotide sequence encoding the AAV5 capsid protein comprises SEQ ID NO: 11. In some embodiments, the polynucleotide sequence encoding the AAV capsid protein comprises a sequence at least 95%, 96%, 97%, 98%, 99%, or 100% identical to SEQ ID NO: 11. In some embodiments, the polynucleotide sequence encoding the AAV5 capsid protein shares at least 95%, 98%, or 100% identity with SEQ ID NO: 11. In some embodiments, the AAV serotype used to infect the eye and/or lacrimal gland is AAV8. In some embodiments, the capsid protein comprises SEQ ID NO: 12. In some embodiments, the AAV capsid protein comprises a sequence at least 95%, 96%, 97%, 98%, 99%, or 100% identical to SEQ ID NO: 12. In some embodiments, the AAV capsid protein shares at least 95%, 98%, or 100% identity to the AAV8 capsid protein (SEQ ID NO: 12). In some embodiments, the polynucleotide encoding the AAV8 capsid protein comprises SEQ ID NO: 13. In some embodiments, the polynucleotide sequence encoding the AAV capsid protein comprises a sequence at least 95%, 96%, 97%, 98%, 99%, or 100% identical to SEQ ID NO: 13. In some embodiments, the polynucleotide sequence encoding the AAV8 capsid protein shares at least 95%, 98%, or 100% identity with SEQ ID NO: 13. In some embodiments, the AAV serotype used to infect the eye and/or lacrimal gland is AAV9. In some embodiments, the AAV capsid protein comprises SEQ ID NO: 14. In some embodiments, the AAV capsid protein comprises a sequence at least 95%, 96%, 97%, 98%, 99%, or 100% identical to SEQ ID NO: 14. In some embodiments, the AAV capsid protein shares at least 95%, 98%, or 100% identity to the AAV9 capsid protein (SEQ ID NO: 14). In some embodiments, the polynucleotide sequence encoding the AAV9 capsid protein comprises SEQ ID NO: 15. In some embodiments, the polynucleotide sequence encoding the AAV capsid protein comprises a sequence at least 95%, 96%, 97%, 98%, 99%, or 100% identical to SEQ ID NO: 15. In some embodiments, the polynucleotide sequence encoding the AAV9 capsid protein shares at least 95%, 98%, or 100% identity with SEQ ID NO: 15.

TABLE 3
AAV Capsid Sequences

| | | Nucleotide |
|---|---|---|
| | Protein | (Open Reading Frame) |
| AAV2 | MAADGYLPDWLEDTLSEGIR | atggctgccgatggttatct |
| VP1 | QWWKLKPGPPPPKPAERHKD | tccagattggctcgaggaca |
| | DSRGLVLPGYKYLGPFNGLD | ctctctctgaaggaataaga |
| | KGEPVNEADAAALEHDKAYD | cagtggtggaagctcaaacc |
| | RQLDSGDNPYLKYNHADAEF | tggcccaccaccaccaaagc |
| | QERLKEDTSFGGNLGRAVFQ | ccgcagagcggcataaggac |
| | AKKRVLEPLGLVEEPVKTAP | gacagcaggggtcttgtgct |
| | GKKRPVEHSPVEPDSSSGTG | tcctgggtacaagtacctcg |
| | KAGQQPARKRLNFGQTGDAD | gacccttcaacggactcgac |
| | SVPDPQPLGQPPAAPSGLGT | aagggagagccggtcaacga |
| | NTMATGSGAPMADNNEGADG | ggcagacgccgcggccctcg |
| | VGNSSGNWHCDSTWMGDRVI | agcacgacaaagcctacgac |
| | TTSTRTWALPTYNNHLYKQI | cggcagctcgacagcggaga |
| | SSQSGASNDNHYFGYSTPWG | caacccgtacctcaagtaca |
| | YFDFNRFHCHFSPRDWQRLI | accacgccgacgcggagttt |
| | NNNWGFRPKRLNFKLFNIQV | caggagcgccttaaagaaga |
| | KEVTQNDGTTTIANNLTSTV | tacgtcttttgggggcaacc |
| | QVFTDSEYQLPYVLGSAHQG | tcggacgagcagtcttccag |
| | CLPPFPADVFMVPQYGYLTL | gcgaaaaagagggttcttga |
| | NNGSQAVGRSSFYCLEYFPS | acctctgggcctggttgagg |
| | QMLRTGNNFTFSYTFEDVPF | aacctgttaagacggctccg |
| | HSSYAHSQSLDRLMNPLIDQ | ggaaaaaagaggccggtaga |
| | YLYYLSRTNTPSGTTTQSRL | gcactctcctgtggagccag |
| | QFSQAGASDIRDQSRNWLPG | actcctcctcgggaaccgga |
| | PCYRQQRVSKTSADNNNSEY | aagggggccagcagcctgca |
| | SWTGATKYHLNGRDSLVNPG | agaaaaagattgaattttgg |
| | PAMASHKDDEEKFFPQSGVL | tcagactggagacgcagact |
| | IFGKQGSEKTNVDIEKVMIT | cagtacctgacccccagcct |
| | DEEEIRTTNPVATEQYGSVS | ctcggacagccaccagcagc |
| | TNLQRGNRQAATADVNTQGV | cccctctggtctgggaacta |
| | LPGMVWQDRDVYLQGPIWAK | atacgatggctacaggcagt |
| | IPHTDGHFHPSPLMGGFGLK | ggcgcaccaatggcagacaa |
| | HPPPQILIKNTPVPANPSTT | taacgagggcgccgacggag |
| | FSAAKFASFITQYSTGQVSV | tgggtaattcctcgggaaat |
| | EIEWELQKENSKRWNPEIQY | tggcattgcgattccacatg |
| | TSNYNKSVNVDFTVDTNGVY | gatgggcgacagagtcatca |
| | SEPRPIGTRYLTRNL | ccaccagcacccgaacctgg |
| | (SEQ ID NO: 6) | gccctgcccacctacaacaa |
| | | ccacctctacaaacaaattt |
| | | ccagccaatcaggagcctcg |
| | | aacgacaatcactactttgg |
| | | ctacagcaccccttgggggt |
| | | attttgacttcaacagattc |
| | | cactgccacttttcaccacg |
| | | tgactggcaaagactcatca |
| | | acaacaactggggattccga |
| | | cccaagagactcaacttcaa |
| | | gctctttaacattcaagtca |
| | | aagaggtcacgcagaatgac |
| | | ggtacgacgacgattgccaa |
| | | taaccttaccagcacggttc |
| | | aggtgtttactgactcggag |
| | | taccagctcccgtacgtcct |
| | | cggctcggcgcatcaaggat |
| | | gcctcccgccgttcccagca |
| | | gacgtcttcatggtgccaca |
| | | gtatggatacctcaccctga |
| | | acaacgggagtcaggcagta |
| | | ggacgctcttcattttactg |
| | | cctggagtactttccttctc |
| | | agatgctgcgtaccggaaac |
| | | aactttaccttcagctacac |
| | | ttttgaggacgttcctttcc |
| | | acagcagctacgctcacagc |
| | | cagagtctggaccgtctcat |
| | | gaatcctctcatcgaccagt |
| | | acctgtattacttgagcaga |
| | | acaaacactccaagtggaac |
| | | caccacgcagtcaaggcttc |
| | | agttttctcaggccggagcg |
| | | agtgacattcgggaccagtc |
| | | taggaactggcttcctggac |
| | | cctgttaccgccagcagcga |
| | | gtatcaaagacatctgcgga |
| | | taacaacaacagtgaatact |
| | | cgtggactggagctaccaag |
| | | taccacctcaatggcagaga |
| | | ctctctggtgaatccgggcc |
| | | cggccatggcaagccacaag |
| | | gacgatgaagaaaagttttt |
| | | tcctcagagcggggttctca |
| | | tctttgggaagcaaggctca |
| | | gagaaaacaaatgtggacat |
| | | tgaaaaggtcatgattacag |
| | | acgaagaggaaatcaggaca |
| | | accaatcccgtggctacgga |
| | | gcagtatggttctgtatcta |
| | | ccaacctccagagaggcaac |
| | | agacaagcagctaccgcaga |
| | | tgtcaacacacaaggcgttc |
| | | ttccaggcatggtctggcag |
| | | gacagagatgtgtaccttca |
| | | ggggcccatctgggcaaaga |
| | | ttccacacacggacggacat |
| | | tttcacccctctcccctcat |
| | | gggtggattcggacttaaac |
| | | accctcctccacagattctc |
| | | atcaagaacaccccggtacc |
| | | tgcgaatccttcgaccacct |
| | | tcagtgcggcaaagtttgct |
| | | tccttcatcacacagtactc |
| | | cacgggacaggtcagcgtgg |
| | | agatcgagtgggagctgcag |
| | | aaggaaaacagcaaacgctg |
| | | gaatcccgaaattcagtaca |
| | | cttccaactacaacaagtct |
| | | gttaatgtggactttactgt |
| | | ggacactaatggcgtgtatt |
| | | cagagcctcgccccattggc |
| | | accagatacctgactcgtaa |
| | | tctgtaa |
| | | (SEQ ID NO: 7) |
| AAV2 | MATGSGAPMADNNEGADGVG | atggctacaggcagtggcgc |
| VP3 | NSSGNWHCDSTWMGDRVITT | accaatggcagacaataacg |
| | STRTWALPTYNNHLYKQISS | agggcgccgacggagtgggt |
| | QSGASNDNHYFGYSTPWGYF | aattcctcgggaaattggca |
| | DFNRFHCHFSPRDWQRLINN | ttgcgattccacatggatgg |
| | NWGFRPKRLNFKLFNIQVKE | gcgacagagtcatcaccacc |
| | VTQNDGTTTIANNLTSTVQV | agcacccgaacctgggccct |
| | FTDSEYQLPYVLGSAHQGCL | gcccacctacaacaaccacc |
| | PPFPADVFMVPQYGYLTLNN | tctacaaacaaatttccagc |
| | GSQAVGRSSFYCLEYFPSQM | caatcaggagcctcgaacga |
| | LRTGNNFTFSYTFEDVPFHS | caatcactactttggctaca |
| | SYAHSQSLDRLMNPLIDQYL | gcaccccttgggggtatttt |
| | YYLSRTNTPSGTTTQSRLQF | gacttcaacagattccactg |
| | SQAGASDIRDQSRNWLPGPC | ccacttttcaccacgtgact |
| | YRQQRVSKTSADNNNSEYSW | ggcaaagactcatcaacaac |
| | TGATKYHLNGRDSLVNPGPA | aactggggattccgacccaa |
| | MASHKDDEEKFFPQSGVLIF | gagactcaacttcaagctct |
| | GKQGSEKTNVDIEKVMITDE | ttaacattcaagtcaaagag |
| | EEIRTTNPVATEQYGSVSTN | gtcacgcagaatgacggtac |
| | LQRGNRQAATADVNTQGVLP | gacgacgattgccaataacc |
| | GMVWQDRDVYLQGPIWAKIP | ttaccagcacggttcaggtg |
| | HTDGHFHPSPLMGGFGLKHP | tttactgactcggagtacca |
| | PPQILIKNTPVPANPSTTFS | gctcccgtacgtcctcggct |
| | AAKFASFITQYSTGQVSVEI | cggcgcatcaaggatgcctc |
| | EWELQKENSKRWNPEIQYTS | ccgccgttcccagcagacgt |
| | NYNKSVNVDFTVDTNGVYSE | cttcatggtgccacagtatg |
| | PRPIGTRYLTRNL | gatacctcaccctgaacaac |
| | (SEQ ID NO: 8) | gggagtcaggcagtaggacg |
| | | ctcttcattttactgcctgg |
| | | agtactttccttctcagatg |
| | | ctgcgtaccggaaacaactt |
| | | taccttcagctacacttttg |
| | | aggacgttcctttccacagc |
| | | agctacgctcacagccagag |
| | | tctggaccgtctcatgaatc |
| | | ctctcatcgaccagtacctg |
| | | tattacttgagcagaacaaa |
| | | cactccaagtggaaccacca |
| | | cgcagtcaaggcttcagttt |
| | | tctcaggccggagcgagtga |
| | | cattcgggaccagtctagga |
| | | actggcttcctggaccctgt |
| | | taccgccagcagcgagtatc |
| | | aaagacatctgcggataaca |
| | | acaacagtgaatactcgtgg |
| | | actggagctaccaagtacca |
| | | cctcaatggcagagactctc |
| | | tggtgaatccgggcccggcc |
| | | atggcaagccacaaggacga |
| | | tgaagaaaagttttttcctc |
| | | agagcggggttctcatcttt |
| | | gggaagcaaggctcagagaa |
| | | aacaaatgtggacattgaaa |
| | | aggtcatgattacagacgaa |
| | | gaggaaatcaggacaaccaa |
| | | tcccgtggctacggagcagt |
| | | atggttctgtatctaccaac |
| | | ctccagagaggcaacagaca |
| | | agcagctaccgcagatgtca |
| | | acacacaaggcgttcttcca |
| | | ggcatggtctggcaggacag |
| | | agatgtgtaccttcaggggc |
| | | ccatctgggcaaagattcca |
| | | cacacggacggacattttca |
| | | cccctctcccctcatgggtg |
| | | gattcggacttaaacaccct |
| | | cctccacagattctcatcaa |
| | | gaacaccccggtacctgcga |
| | | atccttcgaccaccttcagt |
| | | gcggcaaagtttgcttcctt |
| | | catcacacagtactccacgg |
| | | gacaggtcagcgtggagatc |
| | | gagtgggagctgcagaagga |
| | | aaacagcaaacgctggaatc |
| | | ccgaaattcagtacacttcc |
| | | aactacaacaagtctgttaa |
| | | tgtggactttactgtggaca |
| | | ctaatggcgtgtattcagag |
| | | cctcgccccattggcaccag |
| | | atacctgactcgtaatctgt |
| | | aa |
| | | (SEQ ID NO: 9) |
| AAV5 | MSFVDHPPDWLEEVGEGLRE | atgtcttttgttgatcaccc |
| | FLGLEAGPPKPKPNQQHQDQ | tccagattggttggaagaag |
| | ARGLVLPGYNYLGPGNGLDR | ttggtgaaggtcttcgcgag |
| | GEPVNRADEVAREHDISYNE | tttttgggccttgaagcggg |
| | QLEAGDNPYLKYNHADAEFQ | cccaccgaaaccaaaaccca |
| | EKLADDTSFGGNLGKAVFQA | atcagcagcatcaagatcaa |
| | KKRVLEPFGLVEEGAKTAPT | gcccgtggtcttgtgctgcc |
| | GKRIDDHFPKRKKARTEEDS | tggttataactatctcggac |
| | KPSTSSDAEAGPSGSQQLQI | ccggaaacggtctcgatcga |
| | PAQPASSLGADTMSAGGGGP | ggagagcctgtcaacagggc |
| | LGDNNQGADGVGNASGDWHC | agacgaggtcgcgcgagagc |
| | DSTWMGDRVVTKSTRTWVLP | acgacatctcgtacaacgag |
| | SYNNHQYREIKSGSVDGSNA | cagcttgaggcgggagacaa |
| | NAYFGYSTPWGYFDFNRFHS | cccctacctcaagtacaacc |
| | HWSPRDWQRLINNYWGFRPR | acgcggacgccgagtttcag |
| | SLRVKIFNIQVKEVTVQDST | gagaagctcgccgacgacac |
| | TTIANNLTSTVQVFTDDDYQ | atccttcgggggaaacctcg |
| | LPYVVGNGTEGCLPAFPPQV | gaaaggcagtctttcaggcc |
| | FTLPQYGYATLNRDNTENPT | aagaaaagggttctcgaacc |
| | ERSSFFCLEYFPSKMLRTGN | ttttggcctggttgaagagg |
| | NFEFTYNFEEVPFHSSFAPS | gtgctaagacggcccctacc |
| | QNLFKLANPLVDQYLYRFVS | ggaaagcggatagacgacca |
| | TNNTGGVQFNKNLAGRYANT | ctttccaaaaagaaagaagg |
| | YKNWFPGPMGRTQGWNLGSG | ctcggaccgaagaggactcc |
| | VNRASVSAFATTNRMELEGA | aagccttccacctcgtcaga |
| | SYQVPPQPNGMINNLQGSNT | cgccgaagctggacccagcg |
| | YALENTMIFNSQPANPGTTA | gatcccagcagctgcaaatc |
| | TYLEGNMLITSESETQPVNR | ccagcccaaccagcctcaag |
| | VAYNVGGQMATNNQSSTTAP | tttgggagctgatacaatgt |
| | ATGTYNLQEIVPGSVWMERD | ctgcgggaggtggcggccca |
| | VYLQGPIWAKIPETGAHFHP | ttgggcgacaataaccaagg |
| | SPAMGGFGLKHPPPMMLIKN | tgccgatggagtgggcaatg |
| | TPVPGNITSFSDVPVSSFIT | cctcgggagattggcattgc |
| | QYSTGQVTVEMEWELKKENS | gattccacgtggatggggga |
| | KRWNPEIQYTNNYNDPQFVD | cagagtcgtcaccaagtcca |
| | FAPDSTGEYRTTRPIGTRYL | cccgaacctgggtgctgccc |
| | TRPL | agctacaacaaccaccagta |
| | (SEQ ID NO: 10) | ccgagagatcaaaagcggct |
| | | ccgtcgacggaagcaacgcc |
| | | aacgcctactttggatacag |
| | | caccccctgggggtactttg |
| | | actttaaccgcttccacagc |
| | | cactggagcccccgagactg |
| | | gcaaagactcatcaacaact |
| | | actggggcttcagaccccgg |
| | | tccctcagagtcaaaatctt |
| | | caacattcaagtcaaagagg |
| | | tcacggtgcaggactccacc |
| | | accaccatcgccaacaacct |
| | | cacctccaccgtccaagtgt |
| | | ttacggacgacgactaccag |
| | | ctgccctacgtcgtcggcaa |
| | | cgggaccgagggatgcctgc |
| | | cggccttccctccgcaggtc |
| | | tttacgctgccgcagtacgg |
| | | ttacgcgacgctgaaccgcg |
| | | acaacacagaaaatcccacc |
| | | gagaggagcagcttcttctg |
| | | cctagagtactttcccagca |
| | | agatgctgagaacgggcaac |
| | | aactttgagtttacctacaa |
| | | ctttgaggaggtgcccttcc |
| | | actccagcttcgctcccagt |
| | | cagaacctgttcaagctggc |
| | | caacccgctggtggaccagt |
| | | acttgtaccgcttcgtgagc |
| | | acaaataacactggcggagt |
| | | ccagttcaacaagaacctgg |
| | | ccgggagatacgccaacacc |
| | | tacaaaaactggttcccggg |
| | | gcccatgggccgaacccagg |
| | | gctggaacctgggctccggg |
| | | gtcaaccgcgccagtgtcag |
| | | cgccttcgccacgaccaata |
| | | ggatggagctcgagggcgcg |
| | | agttaccaggtgcccccgca |
| | | gccgaacggcatgaccaaca |
| | | acctccagggcagcaacacc |
| | | tatgccctggagaacactat |
| | | gatcttcaacagccagccgg |
| | | cgaacccgggcaccaccgcc |
| | | acgtacctcgagggcaacat |
| | | gctcatcaccagcgagagcg |
| | | agacgcagccggtgaaccgc |
| | | gtggcgtacaacgtcggcgg |
| | | gcagatggccaccaacaacc |
| | | agagctccaccactgccccc |
| | | gcgaccggcacgtacaacct |
| | | ccaggaaatcgtgcccggca |
| | | gcgtgtggatggagagggac |
| | | gtgtacctccaaggacccat |
| | | ctgggccaagatcccagaga |
| | | cgggggcgcactttcacccc |
| | | tctccggccatgggcggatt |
| | | cggactcaaacacccaccgc |
| | | ccatgatgctcatcaagaac |
| | | acgcctgtgcccggaaatat |
| | | caccagcttctcggacgtgc |
| | | ccgtcagcagcttcatcacc |
| | | cagtacagcaccgggcaggt |
| | | caccgtggagatggagtggg |
| | | agctcaagaaggaaaactcc |
| | | aagaggtggaacccagagat |
| | | ccagtacacaaacaactaca |
| | | acgacccccagtttgtggac |
| | | tttgccccggacagcaccgg |
| | | ggaatacagaaccaccagac |
| | | ctatcggaacccgatacctt |
| | | acccgacccctttaa |
| | | (SEQ ID NO: 11) |
| AAV8 | MAADGYLPDWLEDNLSEGIR | atggctgccgatggttatct |
| | EWWALKPGAPKPKANQQKQD | tccagattggctcgaggaca |
| | DGRGLVLPGYKYLGPFNGLD | acctctctgagggcattcgc |
| | KGEPVNAADAAALEHDKAYD | gagtggtgggcgctgaaacc |
| | QQLQAGDNPYLRYNHADAEF | tggagccccgaagcccaaag |
| | QERLQEDTSFGGNLGRAVFQ | ccaaccagcaaaagcaggac |
| | AKKRVLEPLGLVEEGAKTAP | gacggccggggtctggtgct |
| | GKKRPVEPSPQRSPDSSTGI | tcctggctacaagtacctcg |
| | GKKGQQPARKRLNFGQTGDS | gacccttcaacggactcgac |
| | ESVPDPQPLGEPPAAPSGVG | aagggggagcccgtcaacgc |
| | PNTMAAGGGAPMADNNEGAD | ggcggacgcagcggccctcg |
| | GVGSSSGNWHCDSTWLGDRV | agcacgacaaggcctacgac |
| | ITTSTRTWALPTYNNHLYKQ | cagcagctgcaggcgggtga |
| | ISNGTSGGATNDNTYFGYST | caatccgtacctgcggtata |
| | PWGYFDFNRFHCHFSPRDWQ | accacgccgacgccgagttt |
| | RLINNNWGFRPKRLSFKLFN | caggagcgtctgcaagaaga |
| | IQVKEVTQNEGTKTIANNLT | tacgtcttttgggggcaacc |
| | STIQVFTDSEYQLPYVLGSA | tcgggcgagcagtcttccag |
| | HQGCLPPFPADVFMIPQYGY | gccaagaagcgggttctcga |
| | LTLNNGSQAVGRSSFYCLEY | acctctcggtctggttgagg |
| | FPSQMLRTGNNFQFTYTFED | aaggcgctaagacggctcct |
| | VPFHSSYAHSQSLDRLMNPL | ggaaagaagagaccggtaga |
| | IDQYLYYLSRTQTTGGTANT | gccatcaccccagcgttctc |
| | QTLGFSQGGPNTMANQAKNW | cagactcctctacgggcatc |
| | LPGPCYRQQRVSTTTGQNNN | ggcaagaaaggccaacagcc |
| | SNFAWTAGTKYHLNGRNSLA | cgccagaaaaagactcaatt |
| | NPGIAMATHKDDEERFFPSN | ttggtcagactggcgactca |
| | GILIFGKQNAARDNADYSDV | gagtcagttccagaccctca |
| | MLTSEEEIKTTNPVATEEYG | acctctcggagaacctccag |
| | IVADNLQQQNTAPQIGTVNS | cagcgccctctggtgtggga |
| | QGALPGMVWQNRDVYLQGPI | cctaatacaatggctgcagg |
| | WAKIPHTDGNFHPSPLMGGF | cggtggcgcaccaatggcag |
| | GLKHPPPQILIKNTPVPADP | acaataacgaaggcgccgac |
| | PTTFNQSKLNSFITQYSTGQ | ggagtgggtagttcctcggg |
| | VSVEIEWELQKENSKRWNPE | aaattggcattgcgattcca |
| | IQYTSNYYKSTSVDFAVNTE | catggctgggcgacagagtc |
| | GVYSEPRPIGTRYLTRNL | atcaccaccagcacccgaac |
| | (SEQ ID NO: 12) | ctgggccctgcccacctaca |
| | | acaaccacctctacaagcaa |
| | | atctccaacgggacatcggg |
| | | aggagccaccaacgacaaca |
| | | cctacttcggctacagcacc |
| | | ccctgggggtattttgactt |
| | | taacagattccactgccact |
| | | tttcaccacgtgactggcag |
| | | cgactcatcaacaacaactg |
| | | gggattccggcccaagagac |
| | | tcagcttcaagctcttcaac |
| | | atccaggtcaaggaggtcac |
| | | gcagaatgaaggcaccaaga |
| | | ccatcgccaataacctcacc |
| | | agcaccatccaggtgtttac |
| | | ggactcggagtaccagctgc |
| | | cgtacgttctcggctctgcc |
| | | caccagggctgcctgcctcc |
| | | gttcccggcggacgtgttca |
| | | tgattccccagtacggctac |
| | | ctaacactcaacaacggtag |
| | | tcaggccgtgggacgctcct |
| | | ccttctactgcctggaatac |
| | | tttccttcgcagatgctgag |
| | | aaccggcaacaacttccagt |
| | | ttacttacaccttcgaggac |
| | | gtgcctttccacagcagcta |
| | | cgcccacagccagagcttgg |
| | | accggctgatgaatcctctg |
| | | attgaccagtacctgtacta |
| | | cttgtctcggactcaaacaa |
| | | caggaggcacggcaaatacg |
| | | cagactctgggcttcagcca |
| | | aggtgggcctaatacaatgg |
| | | ccaatcaggcaaagaactgg |
| | | ctgccaggaccctgttaccg |
| | | ccaacaacgcgtctcaacga |
| | | caaccgggcaaaacaacaat |
| | | agcaactttgcctggactgc |
| | | tgggaccaaataccatctga |
| | | atggaagaaattcattggct |
| | | aatcctggcatcgctatggc |
| | | aacacacaaagacgacgagg |
| | | agcgtttttttcccagtaac |
| | | gggatcctgatttttggcaa |
| | | acaaaatgctgccagagaca |
| | | atgcggattacagcgatgtc |
| | | atgctcaccagcgaggaaga |
| | | aatcaaaaccactaaccctg |
| | | tggctacagaggaatacggt |
| | | atcgtggcagataacttgca |
| | | gcagcaaaacacggctcctc |
| | | aaattggaactgtcaacagc |
| | | cagggggccttacccggtat |
| | | ggtctggcagaaccgggacg |
| | | tgtacctgcagggtcccatc |
| | | tgggccaagattcctcacac |
| | | ggacggcaacttccacccgt |
| | | ctccgctgatgggcggcttt |
| | | ggcctgaaacatcctccgcc |
| | | tcagatcctgatcaagaaca |
| | | cgcctgtacctgcggatcct |
| | | ccgaccaccttcaaccagtc |
| | | aaagctgaactctttcatca |
| | | cgcaatacagcaccggacag |
| | | gtcagcgtggaaattgaatg |
| | | ggagctgcagaaggaaaaca |
| | | gcaagcgctggaaccccgag |
| | | atccagtacacctccaacta |
| | | ctacaaatctacaagtgtgg |
| | | actttgctgttaatacagaa |
| | | ggcgtgtactctgaaccccg |
| | | ccccattggcacccgttacc |
| | | tcacccgtaatctg |
| | | (SEQ ID NO: 13) |
| AAV9 | MAADGYLPDWLEDNLSEGIR | atggctgccgatggttatct |
| | EWWALKPGAPQPKANQQHQD | tccagattggctcgaggaca |
| | NARGLVLPGYKYLGPGNGLD | accttagtgaaggaattcgc |
| | KGEPVNAADAAALEHDKAYD | gagtggtgggctttgaaacc |
| | QQLKAGDNPYLKYNHADAEF | tggagcccctcaacccaagg |
| | QERLKEDTSFGGNLGRAVFQ | caaatcaacaacatcaagac |
| | AKKRLLEPLGLVEEAAKTAP | aacgctcgaggtcttgtgct |
| | GKKRPVEQSPQEPDSSAGIG | tccgggttacaaataccttg |
| | KSGAQPAKKRLNFGQTGDTE | gacccggcaacggactcgac |
| | SVPDPQPIGEPPAAPSGVGS | aagggggagccggtcaacgc |
| | LTMASGGGAPVADNNEGADG | agcagacgcggcggccctcg |
| | VGSSSGNWHCDSQWLGDRVI | agcacgacaaggcctacgac |
| | TTSTRTWALPTYNNHLYKQI | cagcagctcaaggccggaga |
| | SNSTSGGSSNDNAYFGYSTP | caacccgtacctcaagtaca |
| | WGYFDFNRFHCHFSPRDWQR | accacgccgacgccgagttc |
| | LINNNWGFRPKRLNFKLFNI | caggagcggctcaaagaaga |
| | QVKEVTDNNGVKTIANNLTS | tacgtcttttgggggcaacc |
| | TVQVFTDSDYQLPYVLGSAH | tcgggcgagcagtcttccag |
| | EGCLPPFPADVFMIPQYGYL | gccaaaaagaggcttcttga |
| | TLNDGSQAVGRSSFYCLEYF | acctcttggtctggttgagg |
| | PSQMLRTGNNFQFSYEFENV | aagcggctaagacggctcct |
| | PFHSSYAHSQSLDRLMNPLI | ggaaagaagaggcctgtaga |
| | DQYLYYLSKTINGSGQNQQT | gcagtctcctcaggaaccgg |
| | LKFSVAGPSNMAVQGRNYIP | actcctccgcgggtattggc |
| | GPSYRQQRVSTTVTQNNNSE | aaatcgggtgcacagcccgc |
| | FAWPGASSWALNGRNSLMNP | taaaaagagactcaatttcg |
| | GPAMASHKEGEDRFFPLSGS | gtcagactggcgacacagag |
| | LIFGKQGTGRDNVDADKVMI | tcagtcccagaccctcaacc |
| | TNEEEIKTTNPVATESYGQV | aatcggagaacctcccgcag |
| | ATNHQSAQAQAQTGWVQNQG | ccccctcaggtgtgggatct |
| | ILPGMVWQDRDVYLQGPIWA | cttacaatggcttcaggtgg |
| | KIPHTDGNFHPSPLMGGFGM | tggcgcaccagtggcagaca |
| | KHPPPQILIKNTPVPADPPT | ataacgaaggtgccgatgga |
| | AFNKDKLNSFITQYSTGQVS | gtgggtagttcctcgggaaa |
| | VEIEWELQKENSKRWNPEIQ | ttggcattgcgattcccaat |
| | YTSNYYKSNNVEFAVNTEGV | ggctgggggacagagtcatc |
| | YSEPRPIGTRYLTRNL | accaccagcacccgaacctg |
| | (SEQ ID NO: 14) | ggccctgcccacctacaaca |
| | | atcacctctacaagcaaatc |
| | | tccaacagcacatctggagg |
| | | atcttcaaatgacaacgcct |
| | | acttcggctacagcaccccc |
| | | tgggggtattttgacttcaa |
| | | cagattccactgccacttct |
| | | caccacgtgactggcagcga |
| | | ctcatcaacaacaactgggg |
| | | attccggcctaagcgactca |
| | | acttcaagctcttcaacatt |
| | | caggtcaaagaggttacgga |
| | | caacaatggagtcaagacca |
| | | tcgccaataaccttaccagc |
| | | acggtccaggtcttcacgga |
| | | ctcagactatcagctcccgt |
| | | acgtgctcgggtcggctcac |
| | | gagggctgcctcccgccgtt |
| | | cccagcggacgttttcatga |
| | | ttcctcagtacgggtatctg |
| | | acgcttaatgatggaagcca |
| | | ggccgtgggtcgttcgtcct |
| | | tttactgcctggaatatttc |
| | | ccgtcgcaaatgctaagaac |
| | | gggtaacaacttccagttca |
| | | gctacgagtttgagaacgta |
| | | cctttccatagcagctacgc |
| | | tcacagccaaagcctggacc |
| | | gactaatgaatccactcatc |
| | | gaccaatacttgtactatct |
| | | ctcaaagactattaacggtt |
| | | ctggacagaatcaacaaacg |
| | | ctaaaattcagtgtggccgg |
| | | acccagcaacatggctgtcc |
| | | agggaagaaactacatacct |
| | | ggacccagctaccgacaaca |
| | | acgtgtctcaaccactgtga |
| | | ctcaaaacaacaacagcgaa |
| | | tttgcttggcctggagcttc |
| | | ttcttgggctctcaatggac |
| | | gtaatagcttgatgaatcct |
| | | ggacctgctatggccagcca |
| | | caaagaaggagaggaccgtt |
| | | tctttcctttgtctggatct |
| | | ttaatttttggcaaacaagg |
| | | aactggaagagacaacgtgg |
| | | atgcggacaaagtcatgata |
| | | accaacgaagaagaaattaa |
| | | aactactaacccggtagcaa |
| | | cggagtcctatggacaagtg |
| | | gccacaaaccaccagagtgc |
| | | ccaagcacaggcgcagaccg |
| | | gctgggttcaaaaccaagga |
| | | atacttccgggtatggtttg |
| | | gcaggacagagatgtgtacc |
| | | tgcaaggacccatttgggcc |
| | | aaaattcctcacacggacgg |
| | | caactttcacccttctccgc |
| | | tgatgggagggtttggaatg |
| | | aagcacccgcctcctcagat |
| | | cctcatcaaaaacacacctg |
| | | tacctgcggatcctccaacg |
| | | gccttcaacaaggacaagct |
| | | gaactctttcatcacccagt |
| | | attctactggccaagtcagc |
| | | gtggagatcgagtgggagct |
| | | gcagaaggaaaacagcaagc |
| | | gctggaacccggagatccag |
| | | tacacttccaactattacaa |
| | | gtctaataatgttgaatttg |
| | | ctgttaatactgaaggtgta |
| | | tatagtgaaccccgccccat |
| | | tggcaccagatacctgactc |
| | | gtaatctgt |
| | | (SEQ ID NO: 15) |

In some embodiments, the AAV vector has a serotype selected for high transduction efficiency in the target tissue (e.g., lacrimal gland). Known AAV serotypes include AAV1, AAV2, AAV3, AAV4, AAV5, AAV6, AAV7, AAV8, AAV9, rh10, rh20, rh74 and others. Additionally, various recombinant capsid proteins having point mutations, insertion, and/or deletion are known in the art. In some embodiments, the AAV vector is an AAV vector of serotype AAV1, AAV2, AAV3, AAV4, AAV5, AAV6, AAV7, AAV8, AAV9, rh10, rh20, or rh74. Similarly stated, the AAV vector comprises capsid proteins (VP1, VP2, and/or VP3) homologous the capsid proteins of AAV1, AAV2, AAV3, AAV4, AAV5, AAV6, AAV7, AAV8, AAV9, rh10, rh20, or rh74. In some embodiments, the AAV vector is an AAV vector of serotype AAV2 AAV5, AAV8, or AAV9. In some embodiments, the AAV vector is an AAV vector of serotype AAV2. In some embodiments, the AAV vector is an AAV vector of serotype AAV5. In some embodiments, the AAV vector is an AAV vector of serotype AAV8. In some embodiments, the AAV vector is an AAV vector of serotype AAV9.

In some embodiments, the AAV vector is a hybrid AAV vector (that is, an AAV vector having a polynucleotide genome comprising inverted terminal repeat (ITR) sequences homologous to those of one serotype of AAV but capsid proteins homologous to those of another serotype of AAV). Hybrid AAV are designed by the ITR follow by the capsid (e.g., AAV2/9 refers to an AAV having the ITRs of an AAV2 and the capsid of an AAV9). In some embodiments, the AAV vector is an AAV2/5 vector. In some embodiments, the AAV vector is an AAV2/8 vector. In some embodiments, the AAV vector is an AAV2/9 vector.

An AAV vector of the disclosure comprises two ITRs flanking an expression cassette. The expression cassette includes one or more genes operatively linked to a promoter or promoters. In some embodiments, the promoter is selected from an AAV promoter, such as a p5, p19 or p40 promoter, an adenovirus promoter, such as an adenoviral major later promoter, a cytomegalovirus (CMV) promoter, a papilloma virus promoter, a polyoma virus promoter, a respiratory syncytial virus (RSV) promoter, a sarcoma virus promoter, an SV40 promoter, other viral promoters, an actin promoter, an amylase promoter, an immunoglobulin promoter, a kallikrein promoter, a metallothionein promoter, a heat shock promoter, an endogenous promoter, a promoter regulated by rapamycin or other small molecules, other cellular promoters, and other promoters known to those skilled in the art. In some embodiments, the promoter is an AAV promoter. In some embodiments, the promoter is a CMV promoter. In some embodiments, the promoter is a CAG promoter.

Illustrative methods in production of AAV vectors are provided in Kwon I, Schaffer D V. Pharm Res. 25(3):489-99 (2008); Wu et al. Mol. Ther. 4(3):316-27 (2006); Burger et al. Mol. Ther. 10(2):302-17 (2004); Grimm D, Kay M A. Curr Gene Ther. 3(4):281-304 (2003); Deyle D R, Russell D W. Curr Opin Mol Ther. 11(4): 442-447 (2009); McCarty et al. Gene Ther. 8(16): 1248-54 (2001); and Duan et al. Mol Ther. 4(4):383-91 (2001).

The viral vectors of the disclosure are generally delivered to the subject as a pharmaceutical composition. Pharmaceutical compositions comprise a pharmaceutically acceptable solvent (e.g., water) and one or more excipients. In some embodiments, the pharmaceutical compositions comprise a buffer at about neutral pH (pH 5, 6, 7, 8, or 9). In some embodiments, the pharmaceutical composition comprises phosphate buffered saline (e.g., PBS at pH of about 7). The pharmaceutical compositions may comprise a pharmaceutically acceptable salt. The concentration of the salt may be selected to ensure that the pharmaceutical composition is isotonic to, or nearly isotonic to, the target tissue.

In various embodiments, the pharmaceutical compositions of the disclosure comprise about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, about 5×10¹³ GC/mL, or about 1×10¹⁴ GC/mL of the viral vector (e.g., AAV vector). In various embodiments, the pharmaceutical compositions of the disclosure comprise about 1×10⁸ genome copies per milliliter (GC/mL), about 5×10⁸ GC/mL to about 1×10⁹ GC/mL, about 1×10⁹ GC/mL to about 5×10⁹ GC/mL, about 5×10⁹ GC/mL to about 1×10¹⁰ GC/mL, about 1×10¹⁰ GC/mL to about 5×10¹⁰ GC/mL, about 5×10¹⁰ GC/mL to about 1×10¹¹ GC/mL, about 1×10¹¹ GC/mL to about 5×10¹¹ GC/mL, about 5×10¹¹ GC/mL to about 1×10¹² GC/mL, about 1×10¹² GC/mL to about 5×10¹² GC/mL, about 5×10¹² GC/mL to about 5×10¹³ GC/mL, or about 5×10¹³ GC/mL to about 1×10¹⁴ GC/mL of the viral vector (e.g., AAV vector). In various further embodiments, the pharmaceutical compositions of the disclosure comprise about 5×10⁸ GC/mL to about 5×10⁹ GC/mL, about 5×10⁹ GC/mL to about 5×10¹⁰ GC/mL, about 5×10¹⁰ GC/mL to about 5×10¹¹ GC/mL, about 5×10¹¹ GC/mL to about 5×10¹² GC/mL, or about 5×10¹² GC/mL to about 1×10¹⁴ GC/mL of the viral vector (e.g., AAV vector). In yet further embodiments, the pharmaceutical compositions of the disclosure comprise about 5×10⁸ GC/mL to about 5×10¹⁰ GC/mL, about 5×10¹⁰ GC/mL to about 5×10¹² GC/mL, or about 5×10¹² GC/mL to about 1×10¹⁴ GC/mL of the viral vector (e.g., AAV vector).

In some the pharmaceutical compositions of the disclosure comprise about 1×10¹² GC/mL to about 6.2×10¹² GC/mL of the viral vector (e.g., AAV vector). In some the pharmaceutical compositions of the disclosure comprise about 1×10¹² GC/mL or about 6.2×10¹² GC/mL of the viral vector (e.g., AAV vector).

In some embodiments, the pharmaceutical compositions of the disclosure are administered in a total volume of about 10 μL, about 20 μL, about 30 μL, about 40 μL, about 50 μL, about 60 μL, about 70 μL, about 80 μL, about 90 μL, about 100 μL, 110 μL, about 120 μL, about 130 μL, about 140 μL, about 150 μL, about 160 μL, about 170 μL, about 180 μL, about 190 μL, or about 200 μL. In some embodiments, the pharmaceutical compositions of the disclosure are administered in a total volume of about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to 110 μL, 110 μL to about 120 μL, about 120 μL to about 130 μL, about 130 μL to about 140 μL, about 140 μL to about 150 μL, about 150 μL to about 160 μL, about 160 μL to about 170 μL, about 170 μL to about 180 μL, about 180 μL to about 190 μL, or about 190 μL to about 200 μL. In some embodiments, about 100 μL of the composition is administered. In some embodiments about 50 μL of the composition is administered. In some embodiments about 50 μL to about 100 μL of the composition is administered.

Genome copies per milliliter can be determined by quantitative polymerase change reaction (qPCR) using a standard curve generated with a reference sample having a known concentration of the polynucleotide genome of the virus. For AAV, the reference sample used is often the transfer plasmid used in generation of the AAV vector but other reference samples may be used.

Alternatively or in addition, the concentration of a viral vector can be determined by titering the vector on a cell line. Viral titer is typically expressed as viral particles (vp) per unit volume (e.g., vp/mL). In various embodiments, the pharmaceutical compositions of the disclosure comprise about 1×10⁸ viral particles per milliliter (vp/mL), about 5×10⁸ vp/mL, about 1×10⁹ vp/mL, about 5×10⁹ vp/mL, about 1×10¹⁰ vp/mL, about 5×10¹⁰ vp/mL, about 1×10¹¹ vp/mL, about 5×10¹¹ vp/mL, about 1×10¹² vp/mL, about 5×10¹² vp/mL, about 5×10¹³ vp/mL, or about 1×10¹⁴ vp/mL of the viral vector (e.g., AAV vector). In various further embodiments, the pharmaceutical compositions of the disclosure comprise about 1×10⁸ viral particles per milliliter (vp/mL) to about 5×10⁸ vp/mL, about 5×10⁸ vp/mL to about 1×10⁹ vp/mL, about 1×10⁹ vp/mL to about 5×10⁹ vp/mL, about 5×10⁹ vp/mL to about 1×10¹⁰ vp/mL, about 1×10¹⁰ vp/mL to about 5×10¹⁰ vp/mL, about 5×10¹⁰ vp/mL to about 1×10¹¹ vp/mL, about 1×10¹¹ vp/mL to about 5×10¹¹ vp/mL, about 5×10¹¹ vp/mL to about 1×10¹² vp/mL, about 1×10¹² vp/mL to about 5×10¹² vp/mL, about 5×10¹² vp/mL to about 5×10¹³ vp/mL, or about 5×10¹³ vp/mL to about 1×10¹⁴ vp/mL of the viral vector (e.g., AAV vector).

### Methods of Use

In some embodiments, the disclosure provides methods of treating an ocular surface disorder, cornea disorder, or anterior chamber disorder in a subject. In some embodiments, the disclosure provides methods of treating an ocular surface disorder, cornea disorder, or anterior chamber disorder in a subject a. administering an effective amount of a primary therapeutic agent to the eye or a tissue in fluidic communication with the eye; and b. administering a treatment that increases tear production.

In some embodiments, a tissue in fluidic communication with the eye is the lacrimal gland, the lacrimal duct, the cornea, cornea epithelium, limbal stem cells, conjunctiva, lacrimal puncta, or lacrimal canaliculi. In some embodiments, a tissue in fluidic communication with the eye is one or more of the lacrimal gland, the lacrimal duct, the cornea, cornea epithelium, limbal stem cells, conjunctiva, lacrimal puncta, or lacrimal canaliculi. In some embodiments, a tissue in fluidic communication with the eye is the is the lacrimal gland. In some embodiments, a tissue in fluidic communication with the eye is the is the cornea. In some embodiments, a tissue in fluidic communication with the eye is the is the cornea epithelium. In some embodiments, a tissue in fluidic communication with the eye is the is the limbal stem cells. In some embodiments, a tissue in fluidic communication with the eye is the is the conjunctiva. In some embodiments, a tissue in fluidic communication with the eye is the is the lacrimal puncta. In some embodiments, a tissue in fluidic communication with the eye is the is the lacrimal canaliculi.

In some embodiments, cells within the eye are transduced by the primary therapeutic agent. In some embodiments, acinar cells, ductal cells, and myoepithelial cells are transduced by the primary therapeutic agent. In some embodiments, acinar cells are transduced by the primary therapeutic agent. In some embodiments, ductal cells are transduced by the primary therapeutic agent. In some embodiments, myoepithelial cells are transduced by the primary therapeutic agent.

The methods and compositions of the present disclosure may support, maintain, and/or repair the ocular surface in a subject with an ocular condition. Ocular conditions can be, for example, neurotrophic keratitis, chemical burn of the ocular surface, corneal wound, corneal ulcer, persistent epithelial defect, dry eye disease, herpes simplex viral infection of the trigeminal nerve and/or the eye, varicella zoster virus infection of the trigeminal nerve and/or the eye, and diabetic complications of the corneal nerves. In some embodiments, the ocular condition is neurotrophic keratitis. In some embodiments, the ocular condition is chemical burn of the ocular surface. In some embodiments, the ocular condition is a corneal wound. In some embodiments, the ocular condition is a corneal ulcer. In some embodiments, the ocular condition is a persistent epithelial defect. In some embodiments, the ocular condition is dry eye disease. In some embodiments, the ocular condition is herpes simplex viral infection of the trigeminal nerve and/or the eye. In some embodiments, the ocular condition is varicella zoster virus infection of the trigeminal nerve and/or the eye. In some embodiments, the ocular condition is diabetic complications of the corneal nerves.

Neurotrophic keratitis, or neurotrophic keratopathy, presents as epithelial keratopathy, ulceration, and perforation in the cornea. Neurotrophic keratitis is a degenerative disease of the corneal epithelium resulting from impaired corneal innervation resulting in impaired blinking and decreased tear production. Symptoms include reduced or total loss of corneal sensitivity. Neurotrophic keratitis is diagnosed and staged using one or more of eye exam as described herein, observations of a combination of rose bengal staining of the inferior palpebral conjuctiva, tear mucus viscosity, tear break-up time, fluorescein stain, observation of nonhealing corneal defect, swelling and edematous stroma within the Descemet's membrane, corneal ulcer, corneal perforation, and corneal stromal melting (Sacchetti et al. Clin Ophthalmol.; 8: 571-579 (2014)).

In some embodiments, the disclosure provides a method of maintaining and/or repairing the ocular surface in a subject with a chemical burn of the ocular surface. Chemical burn of the ocular surface typically presents as a sudden onset of severe pain, epiphoria, and blepharospasm. Chemical burn of the ocular surface can be diagnosed through a complete eye examination. Acute periocular signs of injury include periorbital edema and erythema, deepithelialized skin, and loss of eyelashes and eyebrows. Other signs include corneal and conjunctival epithelial defects, chemosis, conjunctival inflammation, limbal ischemia, corneal cloudiness, sterile ulceration, edema, and occasionally perforation. High intraocular pressure may also be a symptom and result from damage and/or inflammation of the trabecular meshwork (Eslani et al. J Ophthalmol.; 2014:196827 (2014)).

In some embodiments, the disclosure provides a method of maintaining and/or repairing the ocular surface in a subject with a corneal wound. Corneal wounds typically present with symptoms of eye pain, tearing, sensitivity to light, and foreign body sensation. Diagnosis is performed using one or more of an eye examination, vision loss assessment, identification of corneal infiltrate or ulcer, identification of hypopyon or hyphema, evidence of penetrating eye injury, presence for foreign bodies in any ocular structures, identification of irregular pupils, and extension of ocular contents. In addition, fluorescein staining may be used to help identify any corneal abrasion (Wipperman et al. Am Fam Physician.; 87(2):114-120 (2013)).

In some embodiments, the disclosure provides a method of maintaining and/or repairing the ocular surface in a subject with a corneal ulcer. Corneal ulcer occurs when there is a failure of healing in the corneal epithelium within a normal two-week period following an injury to the ocular surface. when there is a failure of healing in the corneal epithelium within a normal two-week period following an injury to the ocular surface. Diagnosis is performed using an eye examination as described herein.

Persistent epithelial defect occurs when there is a failure of healing in the corneal epithelium within a normal two-week period following an injury to the ocular surface. Diagnosis is performed using an eye examination as described herein.

In some embodiments, the disclosure provides a method of maintaining and/or repairing the ocular surface in a subject with dry eye disease. Dry eye disease presents with blurry vision, eye irritation, a gritty or foreign body sensation, burning, tearing, photophobia, stinging, or intermittent sharp pain. Dry eye disease may be diagnosed using one or more of an eye examination as described herein, a Schirmer test or tear function index analysis to assess tear production, fluorescein staining to identify corneal epithelial defects, rose bengal staining, lissamine green staining, tear break-up time, a functional visual acuity test, and a tear meniscus assessment (Zeev et al. Clin Ophthalmol.; 8: 581-590 (2014)).

In some embodiments, the disclosure provides a method of maintaining and/or repairing the ocular surface in a subject with a herpes simplex viral infection. Herpes simplex viral infection of the trigeminal nerve and/or the eye, also known as herpes simplex keratitis, presents with symptoms that include redness, discharge, watery eyes, irritation, itching, pain, photophobia, and/or coarse granular spots that form punctuate lesions. Diagnosis is performed using one or more of an eye examination as described herein, slit-lamp examination, lissamine green staining, rose bengal staining, PCR, immunofluorescence antibody assay, and ELISA assay (Azher et al. Clin Ophthalmol. 11: 185-191 (2017)).

In some embodiments, the disclosure provides a method of maintaining and/or repairing the ocular surface in a subject with a varicella zoster virus infection. Varicella zoster virus infection of the trigeminal nerve, also herpes zoster ophthalmicus, has corneal complications that present with varying degrees of decreased vision, pain, and light sensitivity. Diagnosis includes identification of punctate epithelial keratitis, elevated dendritic plaques, granular infiltrates, or other irregularities using slit lamp examination, rose bengal staining, and fluorescein staining (Shaikh et al. Am Fam Physician. 66(9): 1723-1730 (2002)).

In some embodiments, the disclosure provides a method of maintaining and/or repairing the ocular surface in a subject with diabetic complications of the corneal nerves. Diabetic complications of the corneal nerves present with corneal alterations that include increased corneal thickness, epithelial defects, epithelial fragility and recurrent erosions, ulcers, edema, superficial punctate keratitis, delayed and incomplete wound repair, endothelial changes, low tear secretion, dry eye syndrome, and reduced corneal sensitivity (Ljubimov et al. Vision Res.; 139: 138-152 (2017)).

A subject can have, for example, any of the symptoms or ocular disorders described herein.

In some embodiments, the methods described herein result in one or more symptoms of the ocular condition being reduced compared to the symptoms of the ocular condition before administration of the primary therapeutic agent and treatment that increases tear production. As used herein, “symptoms” include any of the diagnostic criteria or symptoms associated with a given ocular condition, including those described herein. In some embodiments, symptoms can be reduced following administration of the compositions of the disclosure. In some embodiments, one or more symptoms of the ocular condition are reduced compared to the symptoms of the ocular condition in an untreated control subject. In some embodiments, one or more symptoms of the ocular condition are reduced compared to the symptoms of the ocular condition in a contralateral eye. By “contralateral eye” it is meant the eye of the subject that is opposite from the eye that has been treated with a composition according to the present disclosure. The contralateral eye can be used as a control for treatment, so long as the subject suffers from bilateral disease, or in the case of a model animal, has been subjected to the experimental protocol leading up to treatment in both eyes.

In some embodiments, the disclosure provides a primary therapeutic agent and a treatment for increasing tear production for use in a method of treating an ocular surface disorder, cornea disorder, or anterior chamber disorder in a subject, the method comprising:
a. administering an effective amount of a primary therapeutic agent to the eye or a tissue in fluidic communication with the eye; and
b. administering a treatment that increases tear production.

In some embodiments, the primary therapeutic agent for use is a composition comprising a viral vector comprising a polynucleotide encoding a gene product, or functional variant thereof. In some embodiments, the treatment that increases tear production is a nicotinic acetylcholine receptor (nAChR) agonist, or a pharmaceutically acceptable salt thereof

In some embodiments, the disclosure provides a primary therapeutic agent for use, or adaptable for use, to treat a subject with an ocular surface disorder, cornea disorder, or anterior chamber disorder. In some embodiments, the primary therapeutic agent for use is a viral vector comprising a polynucleotide encoding a gene product. In some embodiments, the primary therapeutic agent for use is an AAV vector comprising a polynucleotide encoding an NGF protein.

In some embodiments, the disclosure provides a treatment that increases tear production for use, or adaptable for use, to treat a subject with an ocular surface disorder, cornea disorder, or anterior chamber disorder. In some embodiments, the treatment that increases tear production for use is a nicotinic acetylcholine receptor agonist. In some embodiments, the treatment that increases tear production for use is varenicline.

In some embodiments, the disclosure provides a primary therapeutic agent and a treatment for increasing tear production for use in a method of treating an ocular surface disorder, cornea disorder, or anterior chamber disorder in a subject, the method comprising:
a. administering an effective amount of a primary therapeutic agent to the eye or a tissue in fluidic communication with the eye, wherein the primary therapeutic agent is a viral vector comprising a polynucleotide encoding a gene product, and wherein the primary therapeutic agent is administered to at least one lacrimal gland of the subject; and
b. administering a treatment that increases tear production, wherein the treatment that increases tear production is a nicotinic acetylcholine receptor agonist, and wherein the treatment that increases tear production is administered via local nasal administration.

In some embodiments, the disclosure provides a primary therapeutic agent and a treatment for increasing tear production for use in a method of treating an ocular surface disorder, cornea disorder, or anterior chamber disorder in a subject, the method comprising:
a. administering an effective amount of a primary therapeutic agent to the eye or a tissue in fluidic communication with the eye, wherein the primary therapeutic agent is a viral vector comprising a polynucleotide encoding n NGF protein, and wherein the primary therapeutic agent is administered to at least one lacrimal gland of the subject; and
b. administering a treatment that increases tear production, wherein the treatment that increases tear production is varenicline, or a pharmaceutically acceptable salt thereof, and wherein the treatment that increases tear production is administered via local nasal administration.

In some embodiments, the primary therapeutic agent is an rAAV comprising an expression cassette, wherein the expression cassette comprises a polynucleotide encoding a neurotrophic factor. In some embodiments, the primary therapeutic agent is an rAAV comprising an expression cassette, wherein the expression cassette comprises a polynucleotide encoding a neurotrophic factor operably linked to a promoter. In some embodiments, the primary therapeutic agent is an rAAV comprising an AAV capsid and an expression cassette wherein the expression cassette comprises a polynucleotide encoding a neurotrophic factor operably linked to a promoter. In some embodiments, the primary therapeutic agent is an rAAV comprising an AAV capsid and an expression cassette wherein the expression cassette comprises a polynucleotide encoding a neurotrophic factor operably linked to a CAG promoter.

In some embodiments, the primary therapeutic agent is an rAAV comprising an expression cassette, wherein the expression cassette comprises a polynucleotide encoding NGF operatively linked to a promoter. In some embodiments, the primary therapeutic agent is an rAAV comprising an expression cassette, wherein the expression cassette comprises a polynucleotide encoding an NGF protein comprising a sequence that shares at least 95% identity to SEQ ID NO: 2, and wherein the polynucleotide is operatively linked to a promoter. In some embodiments, primary therapeutic agent is an rAAV comprising an expression cassette, wherein the expression cassette comprises a polynucleotide encoding an NGF protein comprising a sequence that shares at least 95% identity to SEQ ID NO: 17, and wherein the polynucleotide is operatively linked to a promoter. In some embodiments, the primary therapeutic agent is an rAAV comprising an expression cassette, wherein the expression cassette comprises a polynucleotide encoding an NGF protein comprising an amino acid sequence that shares at least 95% identity to SEQ ID NO: 1, and wherein the polynucleotide is operatively linked to a promoter. In some embodiments, the primary therapeutic agent is an rAAV comprising an AAV capsid and an expression cassette, wherein the expression cassette comprises a polynucleotide encoding comprising a polynucleotide encoding NGF operatively linked to a CAG promoter.

In some embodiments, the primary therapeutic agent is an rAAV comprising an expression cassette, wherein the expression cassette comprises a polynucleotide encoding GDNF operatively linked to a promoter. In some embodiments, the primary therapeutic agent is an rAAV comprising an expression cassette, wherein the expression cassette comprises a polynucleotide encoding an GDNF protein comprising a sequence that shares at least 95% identity to SEQ ID NO: 4, and wherein the polynucleotide is operatively linked to a promoter. In some embodiments, primary therapeutic agent is an rAAV comprising an expression cassette, wherein the expression cassette comprises a polynucleotide encoding an GDNF protein comprising a sequence that shares at least 95% identity to SEQ ID NO: 18, and wherein the polynucleotide is operatively linked to a promoter. In some embodiments, the primary therapeutic agent is an rAAV comprising an expression cassette, wherein the expression cassette comprises a polynucleotide encoding an GDNF protein comprising an amino acid sequence that shares at least 95% identity to SEQ ID NO: 3, and wherein the polynucleotide is operatively linked to a promoter. In some embodiments, the primary therapeutic agent is an rAAV comprising an AAV capsid and an expression cassette, wherein the expression cassette comprises a polynucleotide encoding comprising a polynucleotide encoding GDNF operatively linked to a CAG promoter.

In some embodiments, the primary therapeutic agent is an rAAV comprising an AAV2 capsid and an expression cassette, wherein the expression cassette comprises a polynucleotide encoding NGF operatively linked to a promoter. In some embodiments, the primary therapeutic agent is an rAAV comprising an AAV2 capsid and an expression cassette, wherein the expression cassette comprises a polynucleotide encoding an NGF protein comprising a sequence that shares at least 95% identity to SEQ ID NO: 2, and wherein the polynucleotide is operatively linked to a promoter. In some embodiments, primary therapeutic agent is an rAAV comprising an AAV2 capsid and an expression cassette, wherein the expression cassette comprises a polynucleotide encoding an NGF protein comprising a sequence that shares at least 95% identity to SEQ ID NO: 17, and wherein the polynucleotide is operatively linked to a promoter. In some embodiments, the primary therapeutic agent is an rAAV comprising an AAV2 capsid and an expression cassette, wherein the expression cassette comprises a polynucleotide encoding an NGF protein comprising an amino acid sequence that shares at least 95% identity to SEQ ID NO: 1, and wherein the polynucleotide is operatively linked to a promoter. In some embodiments, the primary therapeutic agent is an rAAV comprising an AAV2 capsid and an expression cassette, wherein the expression cassette comprises a polynucleotide encoding comprising a polynucleotide encoding NGF operatively linked to a CAG promoter.

In some embodiments, the primary therapeutic agent is an rAAV comprising an AAV2 capsid and an expression cassette, wherein the expression cassette comprises a polynucleotide encoding GDNF operatively linked to a promoter. In some embodiments, the primary therapeutic agent is an rAAV comprising an AAV2 capsid and an expression cassette, wherein the expression cassette comprises a polynucleotide encoding an GDNF protein comprising a sequence that shares at least 95% identity to SEQ ID NO: 4, and wherein the polynucleotide is operatively linked to a promoter. In some embodiments, primary therapeutic agent is an rAAV comprising an AAV2 capsid and an expression cassette, wherein the expression cassette comprises a polynucleotide encoding an GDNF protein comprising a sequence that shares at least 95% identity to SEQ ID NO: 18, and wherein the polynucleotide is operatively linked to a promoter. In some embodiments, the primary therapeutic agent is an rAAV comprising an AAV2 capsid and an expression cassette, wherein the expression cassette comprises a polynucleotide encoding an GDNF protein comprising an amino acid sequence that shares at least 95% identity to SEQ ID NO: 3, and wherein the polynucleotide is operatively linked to a promoter. In some embodiments, the primary therapeutic agent is an rAAV comprising an AAV2 capsid and an expression cassette, wherein the expression cassette comprises a polynucleotide encoding comprising a polynucleotide encoding GDNF operatively linked to a CAG promoter.

In some embodiments, the primary therapeutic agent is an rAAV comprising an AAV5 capsid and an expression cassette, wherein the expression cassette comprises a polynucleotide encoding NGF operatively linked to a promoter. In some embodiments, the primary therapeutic agent is an rAAV comprising an AAV5 capsid and an expression cassette, wherein the expression cassette comprises a polynucleotide encoding an NGF protein comprising a sequence that shares at least 95% identity to SEQ ID NO: 2, and wherein the polynucleotide is operatively linked to a promoter. In some embodiments, primary therapeutic agent is an rAAV comprising an AAV5 capsid and an expression cassette, wherein the expression cassette comprises a polynucleotide encoding an NGF protein comprising a sequence that shares at least 95% identity to SEQ ID NO: 17, and wherein the polynucleotide is operatively linked to a promoter. In some embodiments, the primary therapeutic agent is an rAAV comprising an AAV5 capsid and an expression cassette, wherein the expression cassette comprises a polynucleotide encoding an NGF protein comprising an amino acid sequence that shares at least 95% identity to SEQ ID NO: 1, and wherein the polynucleotide is operatively linked to a promoter. In some embodiments, the primary therapeutic agent is an rAAV comprising an AAV5 capsid and an expression cassette, wherein the expression cassette comprises a polynucleotide encoding comprising a polynucleotide encoding NGF operatively linked to a CAG promoter.

In some embodiments, the primary therapeutic agent is an rAAV comprising an AAV5 capsid and an expression cassette, wherein the expression cassette comprises a polynucleotide encoding GDNF operatively linked to a promoter. In some embodiments, the primary therapeutic agent is an rAAV comprising an AAV5 capsid and an expression cassette, wherein the expression cassette comprises a polynucleotide encoding an GDNF protein comprising a sequence that shares at least 95% identity to SEQ ID NO: 4, and wherein the polynucleotide is operatively linked to a promoter. In some embodiments, primary therapeutic agent is an rAAV comprising an AAV5 capsid and an expression cassette, wherein the expression cassette comprises a polynucleotide encoding an GDNF protein comprising a sequence that shares at least 95% identity to SEQ ID NO: 18, and wherein the polynucleotide is operatively linked to a promoter. In some embodiments, the primary therapeutic agent is an rAAV comprising an AAV5 capsid and an expression cassette, wherein the expression cassette comprises a polynucleotide encoding an GDNF protein comprising an amino acid sequence that shares at least 95% identity to SEQ ID NO: 3, and wherein the polynucleotide is operatively linked to a promoter. In some embodiments, the primary therapeutic agent is an rAAV comprising an AAV5 capsid and an AAV capsid and an expression cassette, wherein the expression cassette comprises a polynucleotide encoding comprising a polynucleotide encoding GDNF operatively linked to a CAG promoter.

In some embodiments, the primary therapeutic agent is an rAAV comprising an AAV9 capsid and an expression cassette, wherein the expression cassette comprises a polynucleotide encoding NGF operatively linked to a promoter. In some embodiments, the primary therapeutic agent is an rAAV comprising an AAV9 capsid and an expression cassette, wherein the expression cassette comprises a polynucleotide encoding an NGF protein comprising a sequence that shares at least 95% identity to SEQ ID NO: 2, and wherein the polynucleotide is operatively linked to a promoter. In some embodiments, primary therapeutic agent is an rAAV comprising an AAV9 capsid and an expression cassette, wherein the expression cassette comprises a polynucleotide encoding an NGF protein comprising a sequence that shares at least 95% identity to SEQ ID NO: 17, and wherein the polynucleotide is operatively linked to a promoter. In some embodiments, the primary therapeutic agent is an rAAV comprising an AAV9 capsid and an expression cassette, wherein the expression cassette comprises a polynucleotide encoding an NGF protein comprising an amino acid sequence that shares at least 95% identity to SEQ ID NO: 1, and wherein the polynucleotide is operatively linked to a promoter. In some embodiments, the primary therapeutic agent is an rAAV comprising an AAV9 capsid and an expression cassette, wherein the expression cassette comprises a polynucleotide encoding comprising a polynucleotide encoding NGF operatively linked to a CAG promoter.

In some embodiments, the primary therapeutic agent is an rAAV comprising an AAV9 capsid and an expression cassette, wherein the expression cassette comprises a polynucleotide encoding GDNF operatively linked to a promoter. In some embodiments, the primary therapeutic agent is an rAAV comprising an AAV9 capsid and an expression cassette, wherein the expression cassette comprises a polynucleotide encoding an GDNF protein comprising a sequence that shares at least 95% identity to SEQ ID NO: 4, and wherein the polynucleotide is operatively linked to a promoter. In some embodiments, primary therapeutic agent is an rAAV comprising an AAV9 capsid and an expression cassette, wherein the expression cassette comprises a polynucleotide encoding an GDNF protein comprising a sequence that shares at least 95% identity to SEQ ID NO: 18, and wherein the polynucleotide is operatively linked to a promoter. In some embodiments, the primary therapeutic agent is an rAAV comprising an AAV9 capsid and an expression cassette, wherein the expression cassette comprises a polynucleotide encoding an GDNF protein comprising an amino acid sequence that shares at least 95% identity to SEQ ID NO: 3, and wherein the polynucleotide is operatively linked to a promoter. In some embodiments, the primary therapeutic agent is an rAAV comprising an AAV9 capsid and an expression cassette, wherein the expression cassette comprises a polynucleotide encoding comprising a polynucleotide encoding GDNF operatively linked to a CAG promoter.

In some embodiments, the primary therapeutic agent is an rAAV comprising an AAV capsid and an expression cassette comprising a polynucleotide sequence comprising SEQ ID NO: 23.

In some embodiments, the primary therapeutic agent is an rAAV comprising an AAV capsid and an expression cassette comprising a polynucleotide sequence comprising SEQ ID NO: 24.

In some embodiments, the disclosure provides a method of treating an ocular surface disorder, cornea disorder, or anterior chamber disorder in a subject, comprising:
(a) administering an effective amount of a primary therapeutic agent to the eye or a tissue in fluidic communication with the eye, wherein the primary therapeutic agent is an rAAV comprising an expression cassette, wherein the expression cassette comprises a polynucleotide encoding a neurotrophic factor, and
(b) administering a treatment that increases tear production.

In some embodiments, the disclosure provides a method of treating an ocular surface disorder, cornea disorder, or anterior chamber disorder in a subject, comprising:
(a) administering an effective amount of a primary therapeutic agent to the eye or a tissue in fluidic communication with the eye, wherein the primary therapeutic agent is an rAAV comprising an expression cassette, wherein the expression cassette comprises a polynucleotide encoding NGF, and
(b) administering a treatment that increases tear production.

In some embodiments, the disclosure provides a method of treating an ocular surface disorder, cornea disorder, or anterior chamber disorder in a subject, comprising:
(a) administering an effective amount of a primary therapeutic agent to the eye or a tissue in fluidic communication with the eye, wherein the primary therapeutic agent is an rAAV comprising an expression cassette, wherein the expression cassette comprises a polynucleotide encoding NGF, and
(b) administering a treatment that increases tear production, wherein the treatment comprises an effective amount of a nicotinic acetylcholine receptor (nAChR) agonist, or a pharmaceutically acceptable salt thereof.

In some embodiments, the disclosure provides a method of treating an ocular surface disorder, cornea disorder, or anterior chamber disorder in a subject, comprising:
(a) administering an effective amount of a primary therapeutic agent to the eye or a tissue in fluidic communication with the eye, wherein the primary therapeutic agent is an rAAV comprising an expression cassette, wherein the expression cassette comprises a polynucleotide encoding NGF, and
(b) administering varenicline, or a pharmaceutically acceptable salt thereof.

In some embodiments, the disclosure provides a method of treating an ocular surface disorder, cornea disorder, or anterior chamber disorder in a subject, comprising:
(a) administering an effective amount of a primary therapeutic agent to the eye or a tissue in fluidic communication with the eye, wherein the primary therapeutic agent is an rAAV comprising an expression cassette, wherein the expression cassette comprises a polynucleotide encoding GDNF, and
(b) administering a treatment that increases tear production.

In some embodiments, the disclosure provides a method of treating an ocular surface disorder, cornea disorder, or anterior chamber disorder in a subject, comprising:
(a) administering an effective amount of a primary therapeutic agent to the eye or a tissue in fluidic communication with the eye, wherein the primary therapeutic agent is an rAAV comprising an expression cassette, wherein the expression cassette comprises a polynucleotide encoding GDNF, and
(b) administering a treatment that increases tear production, wherein the treatment comprises an effective amount of a nicotinic acetylcholine receptor (nAChR) agonist, or a pharmaceutically acceptable salt thereof.

In some embodiments, the disclosure provides a method of treating an ocular surface disorder, cornea disorder, or anterior chamber disorder in a subject, comprising:
(a) administering an effective amount of a primary therapeutic agent to the eye or a tissue in fluidic communication with the eye, wherein the primary therapeutic agent is an rAAV comprising an expression cassette, wherein the expression cassette comprises a polynucleotide encoding GDNF, and
(b) administering varenicline, or a pharmaceutically acceptable salt thereof.

In some embodiments, the disclosure provides a method of treating patient having an ocular surface disorder, cornea disorder, or anterior chamber disorder in a subject, comprising:
a. administering an effective amount of a primary therapeutic agent to the eye or a tissue in fluidic communication with the eye; and
b. administering a treatment that increases tear production, wherein the method results in an increase in expression of the primary therapeutic agent in the tear film in compared to administration of a control treatment.

In some embodiments, the disclosure provides a method of treating patient having an ocular surface disorder, cornea disorder, or anterior chamber disorder in a subject, comprising:
a. administering an effective amount of a gene product to the eye or a tissue in fluidic communication with the eye; and
b. administering a treatment that increases tear production,
wherein the method results in an increase in expression of the gene product in the tear film in compared to administration of a control treatment.

In some embodiments, the disclosure provides a method of treating patient having dry eye disease; bacterial, fungal, or viral keratitis; neurotrophic keratopathy, blepharitis, Sjogren's syndrome; allergic, vernal, viral, and bacterial conjunctivitis; glaucoma; corneal neovascularization; pterygium; corneal dystrophies (keratoconus, Fuch's dystrophy, lattice dystrophy and map-dot-fingerprint dystrophy) or other ocular surface and cornea disorders comprising:
a. administering an effective amount of a primary therapeutic agent to the eye or a tissue in fluidic communication with the eye; and
b. administering a treatment that increases tear production,
wherein the method results in an increase in expression of the primary therapeutic agent in the tear film in compared to administration of a control treatment.

In some embodiments, the disclosure provides a method of treating patient having an ocular surface disorder, cornea disorder, or anterior chamber disorder in a subject, comprising:
a. administering an effective amount of a primary therapeutic agent to the eye or a tissue in fluidic communication with the eye; and
b. administering a treatment that increases tear production,
wherein the method results in about a 5-fold, about 6-fold, about 7-fold, about 8-fold, about 9-fold, or about 10-fold increase in expression of the primary therapeutic agent in the tear film in compared to administration of a control treatment.

In some embodiments, the disclosure provides a method of treating patient having an ocular surface disorder, cornea disorder, or anterior chamber disorder in a subject, comprising:
a. administering an effective amount of a gene product to the eye or a tissue in fluidic communication with the eye; and
b. administering a treatment that increases tear production,
wherein the method results in a 5-fold, about 6-fold, about 7-fold, about 8-fold, about 9-fold, or about 10-fold increase in expression of the gene product in the tear film in compared to administration of a control treatment.

In some embodiments, the disclosure provides a method of treating patient having dry eye disease; bacterial, fungal, or viral keratitis; neurotrophic keratopathy, blepharitis, Sjogren's syndrome; allergic, vernal, viral, and bacterial conjunctivitis; glaucoma; corneal neovascularization; pterygium; corneal dystrophies (keratoconus, Fuch's dystrophy, lattice dystrophy and map-dot-fingerprint dystrophy) or other ocular surface and cornea disorders comprising:
a. administering an effective amount of a gene product to the eye or a tissue in fluidic communication with the eye; and
b. administering a treatment that increases tear production,
wherein the method results in a 5-fold, about 6-fold, about 7-fold, about 8-fold, about 9-fold, or about 10-fold increase in expression of the gene product in the tear film in compared to administration of a control treatment.

In some embodiments, the disclosure provides a method of treating patient having dry eye disease; bacterial, fungal, or viral keratitis; neurotrophic keratopathy, blepharitis, Sjogren's syndrome; allergic, vernal, viral, and bacterial conjunctivitis; glaucoma; corneal neovascularization; pterygium; corneal dystrophies (keratoconus, Fuch's dystrophy, lattice dystrophy and map-dot-fingerprint dystrophy) or other ocular surface and cornea disorders comprising:
a. administering an effective amount of a primary therapeutic agent to the eye or a tissue in fluidic communication with the eye; and
b. administering a treatment that increases tear production,
wherein the method results in a 5-fold, about 6-fold, about 7-fold, about 8-fold, about 9-fold, or about 10-fold increase in expression of the primary therapeutic agent in the tear film in compared to administration of a control treatment.

In some embodiments, the primary therapeutic agent comprises a viral vector comprising a polynucleotide encoding a gene product. In some embodiments, the control treatment is administration of the primary therapeutic agent alone. In some embodiments, the control treatment is a control solution (e.g., phosphate buffered saline). In some embodiments, the control treatment is no administration.

### Indicators of Effective Treatment

The characterization of the trigeminal parasympathetic pathway and its importance to methods described herein are briefly discussed here. The trigeminal nerve comprises the ophthalmic nerve, the maxillary nerve, and the mandibular nerve. The ophthalmic and maxillary branches of the trigeminal nerve form the afferent paths. The nasal mucosal epithelium and nociceptive nerves are lined with receptors that initiate the afferent limbs of reflexes within the nose. The efferent paths of the reflex proceed from the superior salivary nucleus along the facial nerve (intermedius) to the geniculate ganglion and from there through the greater superficial petrosal nerve via the sphenopalatine ganglion to the glands of the lacrimal functional unit (lacrimal glands, meibomian glands, and goblet cells). See Wong et al. Neuroscience Letters 649:14-19 (2017). Healthy tear film composition is complex and consists of a superficial lipid layer, an intermediary mucin layer, and a deep mucin layer. Each of the components of these layers originates from different glands; the meibomian gland (lipid layer), the lacrimal gland (aqueous layer), and the goblet cells (mucin layer). Neuro-anatomical studies in animals illustrate that the trigeminal-parasympathetic pathway innervates each of these glands. The present disclosure provides methods comprising stimulating the trigeminal-parasympathetic pathway as a means to excrete the tear film components.

In some of the embodiments described herein, the method may comprise activating the nasolacrimal reflex. In some of the embodiments described herein, the method may comprise activating the trigeminal nerve. In some of the embodiments described herein, the method may comprise activating the anterior ethmoidal nerve.

In some of the embodiments described herein, the methods comprise improving tear film homeostasis. In some of the embodiments described herein, the methods comprise re-establishing tear film homeostasis. In some of the embodiments, the subject in need thereof lacks tear film homeostasis or has impaired tear film homeostasis.

A variety of tests are available to evaluate a subject's condition before, during, and after treatment with any of the methods disclosed herein. In some of the embodiments disclosed herein, the effective treatment of the subject is indicated by one or more of the tests selected from the group consisting of a) Eye Dryness score test on a visual analog scale, b) Schirmer's test, c) Corneal Fluorescein Staining test, and d) Ocular Surface Disease Index test. Tests to evaluate the signs and symptoms of an ocular condition may be administered under standardized or reproducible conditions in order to obtain a subject's test score. Conditions include exposing the subject to an environment artificially created to adversely challenge the subject or where the environment (temperature, humidity, air flow) is monitored and carefully controlled.

The next sections provide further details on the Eye Dryness Score test on a visual analog scale, Schirmer's test, Corneal Fluorescein Staining test, and Ocular Surface Disease Index test.

### Eye Dryness Score Test

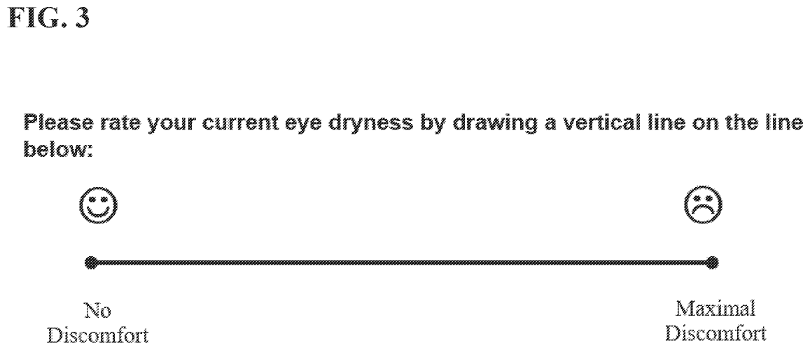

An Eye Dryness Score test on a visual analog scale may be used to evaluate dry eye disease, tearing levels, or ocular discomfort in a subject. The test involves the use of a visual analog scale and comprises a 100 mm horizontal line where one endpoint at 0 is labeled “no discomfort” and the other endpoint at 100 is labeled “maximal discomfort”. FIG. 3 shows an example of the visual analog scale (not shown at actual scale). The subject is asked to rate their ocular symptoms due to eye dryness by placing a vertical mark on the horizontal line to indicate their level of discomfort. The Eye Dryness score is then obtained by identifying where the subject's response is on the 100 mm scale.

The Eye Dryness score, measured in mm, can be used to evaluate the severity of the ocular symptom, and the effectiveness of a particular treatment of a subject. This test has the advantage of being administered to the subject as frequently as every 5 minutes, which allows the test administrator to closely monitor the changes in the subject's symptoms over time. Higher numbers indicate more discomfort for an ocular symptom compared to lower numbers, which indicate relatively lower levels of discomfort. A decrease in the Eye Dryness score is evidence that the treatment is effective in treating dry eye disease, increasing tear production, or improving ocular discomfort. Reduction in Eye Dryness scores over time is evidence of a reduction in or alleviation of the ocular symptom and generally indicates an improvement in the subject's condition.

In some of the embodiments disclosed herein, effective treatment is indicated by a statistically significant decrease in the subject's Eye Dryness score, and wherein the statistically significant decrease in the subject's Eye Dryness score is determined after administration to the subject of the first dose, or the optionally one or more subsequent doses, of the primary therapeutic agent, and the first dose, or the optionally one or more subsequent doses, of the treatment that increases tear production, wherein the subject's Eye Dryness score is compared to a) an Eye Dryness score of the subject prior to administration of the first dose of the primary therapeutic agent, and the first dose of the treatment that increases tear production; b) an Eye Dryness score of a subject administered a control; or c) an Eye Dryness score of a subject administered a comparator compound.

As used herein, the term “statistically significant” refers to a method of analysis selected by a person of skill in the art based upon study design and the data type generated to assess the difference observed between two or more groups, wherein the analysis determines if the difference is not random or due strictly to chance.

In some of the embodiments disclosed herein, the subject's Eye Dryness score is compared to an Eye Dryness score of the subject prior to administration of the first dose of the primary therapeutic agent, and the first dose of the treatment that increases tear production.

In some of the embodiments disclosed herein, the subject's Eye Dryness score is compared to an Eye Dryness score of a subject administered a control.

In some of the embodiments disclosed herein, the subject's Eye Dryness score is compared to an Eye Dryness score of a subject administered a comparator compound.

In some of the embodiments disclosed herein, the subject's Eye Dryness score is compared to an Eye Dryness score in a contralateral eye.

In some embodiments, a statistically significant decrease in a subject's Eye Dryness score is observed in a subject administered a primary therapeutic agent and a treatment that increases tear production as described herein compared to a control.

In some of the embodiments described herein, the statistically significant decrease in the subject's Eye Dryness Score is at least 5%, 10%, 15%, 20%, 25%, 50%, 75%, 80%, 90%, 95%, 100%, 125%, 150%, 200%, 250%, 300%, or 350%.

In some embodiments, a statistically significant decrease in a subject's Eye Dryness score is observed in a subject administered a primary therapeutic agent and a treatment that increases tear production as described herein compared to a control, wherein the statistically significant decrease in the subject's Eye Dryness Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's Eye Dryness score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm. In some embodiments, a statistically significant decrease in a subject's Eye Dryness score is observed in a subject administered a primary therapeutic agent and a treatment that increases tear production as described herein compared to a control, wherein the statistically significant decrease in the subject's Eye Dryness Score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant decrease in the subject's Eye Dryness score is between 3 mm and 10 mm, between 3 mm and 20 mm, between 3 mm and 25 mm, between 3 mm and 30 mm, between 3 mm and 35 mm, between 3 mm and 40 mm, between 3 mm and 45 mm, between 3 mm and 50 mm, between 5 mm and 10 mm, between 5 mm and 20 mm, between 5 mm and 25 mm, between 5 mm and 30 mm, between 5 mm and 35 mm, between 5 mm and 40 mm, between 5 mm and 45 mm, between 5 mm and 50 mm, between 10 mm and 15 mm, between 10 mm and 20 mm, between 10 mm and 25 mm, between 10 mm and 30 mm, between 10 mm and 35 mm, between 10 mm and 40 mm, between 10 mm and 45 mm, between 10 mm and 50 mm, between 15 mm and 20 mm, between 20 mm and 30 mm, between 25 mm and 35 mm, between 30 mm and 40 mm, between 30 mm and 45 mm, or between 30 mm and 50 mm.

In some of the embodiments described herein, the statistically significant decrease in the subject's Eye Dryness Score is characterized by a p value of 0.05 or less, 0.01 or less, 0.005 or less, or 0.001 or less. In some of the embodiments described herein, the statistically significant decrease in the subject's Eye Dryness Score is characterized by a p value of 0.05 or less. In some of the embodiments described herein, the statistically significant decrease in the subject's Eye Dryness Score is characterized by a p value of 0.01 or less.

In some of the embodiments described herein, the statistically significant decrease in the subject's Eye Dryness Score is within 5 minutes, within 10 minutes, within 15 minutes, within 20 minutes, within 30 minutes, within 45 minutes or within 60 minutes of administration of the first dose of an effective amount of the primary therapeutic agent, and, optionally, the first dose of the treatment that increases tear production. In some of the embodiments described herein, the statistically significant decrease in the subject's Eye Dryness Score is within 5 minutes or 10 minutes of the first dose of an effective amount of the primary therapeutic agent.

In some of the embodiments described herein, the statistically significant decrease in the subject's Eye Dryness Score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months of administration of the first dose of an effective amount of the primary therapeutic agent, and, optionally, the first dose of the treatment that increases tear production.

In some embodiments, the decrease in a subject's Eye Dryness Score persists for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant decrease in the subject's Eye Dryness score is based on the subject's Eye Dryness score determined after administering the first dose of the primary therapeutic agent.

In some of the embodiments described herein, the statistically significant decrease is based on the subject's Eye Dryness score determined after administering one or more subsequent doses of the primary therapeutic agent, and optionally the first or the one or more subsequent doses of the treatment that increases tear production.

### Schirmer Score Test

Dry eye disease affects tear volume and tear production. A Schirmer's test may be used to evaluate tear production and assess the severity of dry eye disease, insufficient tearing, or ocular discomfort in a subject. The test measures the amount of tears produced in each eye. The test typically involves first placing an anesthetic into one or both of the subject's eyes. These drops prevent the eyes from watering in reaction to the test strips. Then, the test administrator places a piece of filter paper inside one or both lower eyelids and the person closes their eyes. After 5 minutes, the test administrator removes the filter paper and assesses how far the tears have travelled on the paper. The Schirmer's test may be administered to one or both eyes.

In general, the smaller the amount of moisture on the paper, the fewer tears that person has produced. In healthy eyes, each strip of paper typically contains more than 10 millimeters of moisture. A Schirmer's score of less than 10 millimeters of moisture may indicate one or more of the following conditions including dry eye disease, abnormally low tearing, or ocular discomfort.

A Schirmer's test may be used to evaluate the effectiveness of a particular treatment in a subject and may be administered multiple times to monitor any change in the severity of the subject's symptoms over a period of time. An increase in Schirmer's scores over time in an subject being treated for dry eye disease, insufficient tearing, or ocular discomfort is evidence for an increase in tear volume or tear production and generally indicates improvement in the subject's condition. An increase in the Schirmer's score over time is evidence that treatment is effective in treating dry eye disease, increasing tear production, or improving ocular discomfort.

In some of the embodiments disclosed herein, effective treatment is indicated by a statistically significant increase in the subject's Schirmer's score, and wherein the statistically significant increase in the subject's Schirmer's score is determined after administration to the subject of the first dose, or the optionally one or more subsequent doses, of the primary therapeutic agent, and the first dose, or the optionally one or more subsequent doses, of the treatment that increases tear production, wherein the subject's Schirmer's score is compared to a) a Schirmer's score of the subject prior to administration of the first dose of the primary therapeutic agent, and the first dose of the treatment that increases tear production; b) a Schirmer's score of a subject administered a control; or c) a Schirmer's score of a subject administered a comparator compound.

In some of the embodiments disclosed herein, the subject's Schirmer's score is compared to a Schirmer's score of the subject prior to administration of the first dose of the primary therapeutic agent, and the first dose of the treatment that increases tear production. In some of the embodiments disclosed herein, the subject's Schirmer's score is compared to a Schirmer's score of a subject administered a control. In some of the embodiments disclosed herein, the subject's Schirmer's score is compared to a Schirmer's score of a subject administered a comparator compound.

In some embodiments, a statistically significant increase in a subject's Schirmer's score is observed in a subject administered a primary therapeutic agent and a treatment that increases tear production as described herein compared to a control.

In some of the embodiments described herein, the statistically significant increase in the subject's Schirmer's score is at least 5%, 10%, 15%, 20%, 25%, 50%, 75%, 80%, 90%, 95%, 100%, 125%, 150%, 200%, 250%, 300%, or 350%. In some of the embodiments described herein, the increase in the subject's Schirmer's score is at least 100%, 200%, or 300%. In some embodiments, a statistically significant increase in a subject's Schirmer's score is observed in a subject administered a primary therapeutic agent and a treatment that increases tear production as described herein compared to a control, wherein the statistically significant increase in the subject's Schirmer's Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant increase in the subject's Schirmer's score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant increase in the subject's Schirmer's score is between 3 mm and 5 mm, between 3 mm and 10 mm, between 3 mm and 15 mm, between 3 mm and 20 mm, between 3 mm and 25 mm, between 3 mm and 30 mm, between 5 mm and 10 mm, between 5 mm and 15 mm, between 5 mm and 20 mm, between 5 mm and 25 mm, between 5 mm and 30 mm, between 10 mm and 15 mm, between 10 mm and 20 mm, between 10 mm and 25 mm, between 10 mm and 30 mm, 15 mm and 20 mm, between 15 mm and 25 mm, between 15 mm and 30 mm, between 20 mm and 25 mm, or between 20 mm and 30 mm.

In some embodiments, a statistically significant increase in a subject's Schirmer's score is observed in a subject administered a primary therapeutic agent and a treatment that increases tear production as described herein compared to a control, wherein the statistically significant increase in the subject's Schirmer's score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant increase in the subject's Schirmer's score is characterized by a p value of 0.05 or less, 0.01 or less, 0.005 or less, or 0.001 or less.

In some of the embodiments described herein, the statistically significant increase in the subject's Schirmer's score is within 5 minutes, within 10 minutes, within 15 minutes, within 20 minutes, within 30 minutes, within 45 minutes or within 60 minutes of administration of the first dose of an effective amount of the primary therapeutic agent, and, optionally, the first dose of the treatment that increases tear production. In some of the embodiments described herein, the statistically significant decrease in the subject's Schirmer's score is within 5 minutes of the first dose of an effective amount of the primary therapeutic agent.

In some of the embodiments described herein, the statistically significant increase in the subject's Schirmer's score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months of administration of the first dose of an effective amount of the primary therapeutic agent, and, optionally, the first dose of the treatment that increases tear production.

In some of the embodiments described herein, the statistically significant increase in the subject's Schirmer's score is within 1 minute, 2 minutes, 5 minutes, when measured acutely, or within 1 day, within 2 days, within 3 days, within 4 days, within 1 week, within 2 weeks, within 3 weeks, within 4 weeks, within 1 month, within 2 months, within 3 months, within 4 months, within 5 months or within 6 months when measured chronically after treatment according to the methods or compositions disclosed herein.

In some embodiments, the increase in a subject's Schirmer's score persists for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some embodiments, the increase in a subject's Schirmer's score persists for 1 minute, 2 minutes, 3 minutes, or at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant increase in the subject's Schirmer's score is based on the subject's Schirmer's score determined after administering the first dose of the primary therapeutic agent.

In some of the embodiments described herein, the statistically significant increase is based on the subject's Schirmer's score determined after administering one or more subsequent doses of the primary therapeutic agent, and optionally the first or the one or more subsequent doses of the treatment that increases tear production.

### Corneal Fluorescein Staining Test

Corneal surface changes are associated with insufficient tear flow and excessive dryness, as well as dry eye disease and ocular discomfort. Corneal surface changes may include disruption of the mucin coating protecting the surface epithelial cells, and/or damage to the epithelial cell walls.

Corneal Fluorescein Staining test is a diagnostic test for determining corneal surface health and can indicate areas of damage on the corneal surface. The normal corneal surface does not take up water-soluble dyes instilled into the tear film. However, damaged epithelial cells allow water-soluble dyes to diffuse into the surface cells. Fluorescein, which stains damaged epithelial cells, may be visualized on the corneal surface indicating damage as a result of desiccation on the corneal surface.

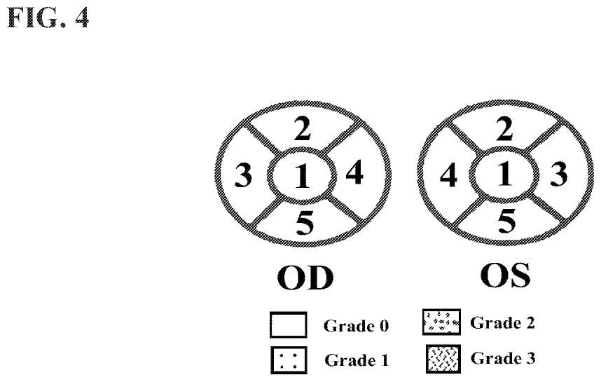

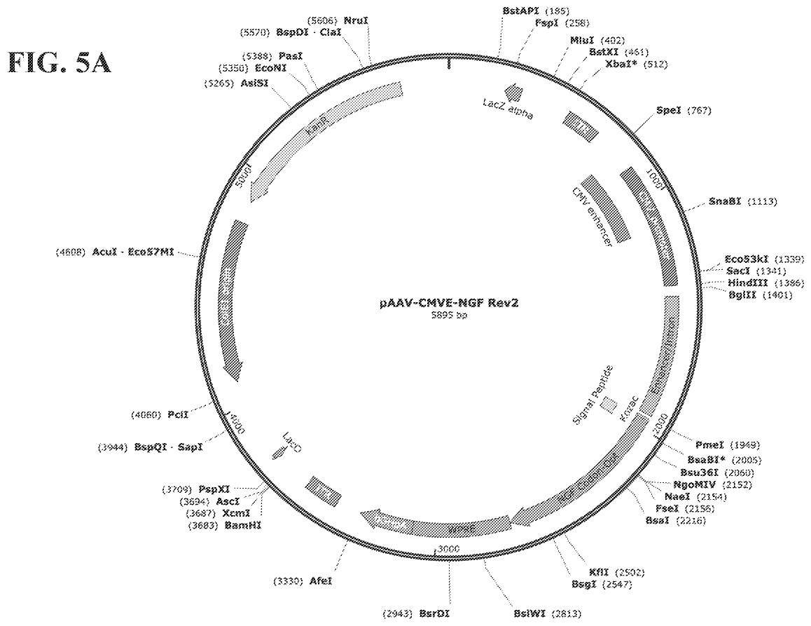

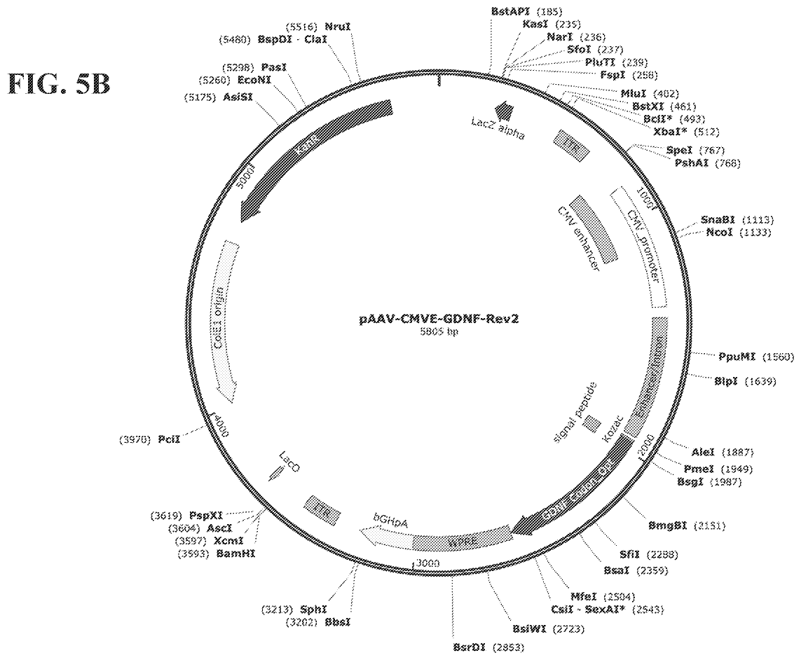

To administer the Corneal Fluorescein Staining test, fluorescein is applied to one or both eyes. Fluorescein is allowed to penetrate and stain the area between surface cells. Devitalized cells and strands of devitalized surface tissue (filaments) can be visualized with this stain. A test administrator then uses a corneal surface scoring system has been developed to rate the severity of damage observed. This scoring system is useful for monitoring dry eye treatment over time. FIG. 4 depicts the NEI/Industry Workshop Scale used in the scoring system. Other equivalent standardized scoring systems may be used. A test administrator grades the areas of the corneal surface that are damaged and calculates the corneal score reflecting the severity of damage to the corneal surface.

A test administrator may use the corneal score to evaluate the effectiveness of a particular treatment in a subject. The test may be administered multiple times to monitor any change in the severity of the subject's ocular surface over a period of time. In general, higher numbers indicate more damage to the corneal surface compared to lower numbers, which indicate lower levels of damage to the corneal surface. Reduction in corneal scores over time is evidence of a reduction in the damage to corneal surface. The decrease in corneal scores generally indicates and improvement in the subject's condition. The decrease in corneal scores over time is also evidence that the treatment is effective in treating dry eye disease, increasing tear production, or improving ocular discomfort.

In some of the embodiments disclosed herein, effective treatment is indicated by a statistically significant decrease in the subject's corneal score, and wherein the statistically significant decrease in the subject's corneal score is determined after administration to the subject of the first dose, or the optionally one or more subsequent doses, of the primary therapeutic agent, and the first dose, or the optionally one or more subsequent doses, of the treatment that increases tear production, wherein the subject's corneal score is compared to a) a corneal score of the subject prior to administration of the first dose of the primary therapeutic agent, and the first dose of the treatment that increases tear production; b) a corneal score of an subject administered a control; or c) a corneal score of an subject administered a comparator compound.

In some of the embodiments disclosed herein, the subject's corneal score is compared to a corneal score of the subject prior to administration of the first dose of the primary therapeutic agent, and the first dose of the treatment that increases tear production. In some of the embodiments disclosed herein, the subject's corneal score is compared to a corneal score of a subject administered a control. In some of the embodiments disclosed herein, the subject's corneal score is compared to a corneal score of a subject administered a comparator compound.

In some embodiments, a statistically significant decrease in a subject's corneal score is observed in a subject administered a primary therapeutic agent and a treatment that increases tear production as described herein compared to a control.

In some of the embodiments described herein, the statistically significant decrease in the subject's corneal score is at least 5%, 10%, 15%, 20%, 25%, 50%, 75%, 80%, 90%, 95%, 100%, 125%, 150%, 200%, 250%, 300%, or 350%.

In some embodiments, a statistically significant decrease in a subject's corneal score is observed in a subject administered a primary therapeutic agent and a treatment that increases tear production as described herein compared to a control, wherein the statistically significant decrease in the subject's corneal score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's corneal score is characterized by a p value of 0.05 or less, 0.01 or less, 0.005 or less, or 0.001 or less.

In some of the embodiments described herein, the statistically significant decrease in the subject's corneal score is within 5 minutes, within 10 minutes, within 15 minutes, within 20 minutes, within 30 minutes, within 45 minutes or within 60 minutes of administration of the first dose of an effective amount of the primary therapeutic agent, and, optionally, the first dose of the treatment that increases tear production. In some of the embodiments described herein, the statistically significant decrease in the subject's corneal score is within 5 minutes of the first dose of an effective amount of the primary therapeutic agent.

In some embodiments, the decrease in a subject's corneal score persists for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, the statistically significant decrease in the subject's corneal score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months of administration of the first dose of an effective amount of the primary therapeutic agent, and, optionally, the first dose of the treatment that increases tear production. In some of the embodiments described herein, wherein the statistically significant decrease in the subject's corneal score is based on the subject's corneal score determined after administering the first dose of the primary therapeutic agent.

In some of the embodiments described herein, the statistically significant decrease is based on the subject's corneal score determined after administering one or more subsequent doses of the primary therapeutic agent, and optionally the first or the one or more subsequent doses of the treatment that increases tear production.

### Ocular Surface Disease Index Test

Subjects treated for dry eye disease, increasing tear production, or improving ocular discomfort can provide important information used to diagnose their condition and determine the severity of dry eye symptoms through questionnaires. A well-designed questionnaire can be validated for reproducibility and should consist of relevant questions that elicit responsive answers. One example of a dry-eye questionnaire is the Ocular Surface Disease Index (OSDI), a 12-question survey for dry-eye patients that's been shown to be a reliable and valid instrument for directly assessing symptom frequency. The ocular symptoms that are evaluated include, but are not limited to, burning/stinging, itching, foreign body sensation, eye discomfort, eye dryness, photophobia, and pain. Most people are familiar with questionnaires and understand that they are an efficient way for a health provider to gather information. Questionnaires also reduce bias, as there are no verbal or visual cues to inadvertently influence the respondent. The test administrator collects the response from the subject and calculates an OSDI based on the subject's answers to the questions.

The OSDI score can be used to evaluate the severity of the ocular symptom, and the effectiveness of a particular treatment of a subject. Higher numbers indicate a higher severity of dry eye disease. Reduction in OSDI scores over time is evidence of a reduction in or alleviation of the ocular symptoms and generally indicates an improvement in the subject's condition. A decrease in the OSDI score is also evidence that the treatment is effective in treating dry eye disease, increasing tear production, or improving ocular discomfort.

In some of the embodiments disclosed herein, effective treatment is indicated by a statistically significant decrease in the subject's OSDI score, and wherein the statistically significant decrease in the subject's OSDI score is determined after administration to the subject of the first dose, or the optionally one or more subsequent doses, of the primary therapeutic agent, and the first dose, or the optionally one or more subsequent doses, of the treatment that increases tear production, wherein the subject's OSDI score is compared to a) an OSDI score of the subject prior to administration of the first dose of the primary therapeutic agent, and the first dose of the treatment that increases tear production; b) an OSDI score of an subject administered a control; or c) an OSDI score of a subject administered a comparator compound.

In some of the embodiments disclosed herein, the subject's OSDI score is compared to an OSDI score of the subject prior to administration of the first dose of the primary therapeutic agent, and the first dose of the treatment that increases tear production. In some of the embodiments disclosed herein, the subject's OSDI score is compared to an OSDI score of a subject administered a control. In some of the embodiments disclosed herein, the subject's OSDI score is compared to an OSDI score of a subject administered a comparator compound.

In some embodiments, a statistically significant decrease in a subject's OSDI score is observed in a subject administered a primary therapeutic agent and a treatment that increases tear production as described herein compared to a control.

In some of the embodiments described herein, the statistically significant decrease in the subject's OSDI score is at least 5%, 10%, 15%, 20%, 25%, 50%, 75%, 80%, 90%, 95%, 100%, 125%, 150%, 200%, 250%, 300%, or 350%.

In some embodiments, a statistically significant decrease in a subject's OSDI score is observed in a subject administered a primary therapeutic agent and a treatment that increases tear production as described herein compared to a control, wherein the statistically significant decrease in the subject's OSDI Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's OSDI score is characterized by a p value of 0.05 or less, 0.01 or less, 0.005 or less, or 0.001 or less.

In some of the embodiments described herein, the statistically significant decrease in the subject's OSDI score is within 5 minutes, within 10 minutes, within 15 minutes, within 20 minutes, within 30 minutes, within 45 minutes or within 60 minutes of administration of the first dose of an effective amount of the primary therapeutic agent, and, optionally, the first dose of the treatment that increases tear production. In some of the embodiments described herein, the statistically significant decrease in the subject's OSDI score is within 5 minutes of the first dose of an effective amount of the primary therapeutic agent.

In some of the embodiments described herein, the statistically significant decrease in the subject's OSDI score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months of administration of the first dose of an effective amount of the primary therapeutic agent, and, optionally, the first dose of the treatment that increases tear production.

In some embodiments, the decrease in a subject's OSDI score persists for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant decrease in the subject's OSDI score is based on the subject's OSDI score determined after administering the first dose of the primary therapeutic agent.

In some of the embodiments described herein, the statistically significant decrease is based on the subject's OSDI score determined after administering one or more subsequent doses of the primary therapeutic agent, and optionally the first or the one or more subsequent doses of the treatment that increases tear production.

### Maintenance of Effective Treatment Over Time

The present disclosure provides for effective treatment over a period of time where a statistically significant improvement in a subject's score is maintained. The term “maintained” as used in the present disclosure and as it relates to the maintenance of a statistically significant improvement in a subject's score (EDS, Schirmer, corneal, or OSDI) refers to the statistically significant improvement not diminishing below a certain threshold over time. A statistically significant improvement can be maintained even if, at a later point in time, the subject's score changes. An improvement after a first dose of the primary therapeutic agent, and, optionally, the first dose of the treatment that increases tear production, can be maintained without additional dosing or after one or more subsequent doses.

For example, an Eye Dryness score being “maintained within 10% means” that the decrease in subject's Eye Dryness score does not diminish by more than 10% during the specified time. A further improvement in the subject's Eye Dryness score would also be considered maintenance of the statistically significant improvement (e.g., if the Eye Dryness score further improved by 15% during the specified time, this would be considered “maintained within 10%”).

In another example, if there is a statistically significant decrease (improvement) in a subject's Eye Dryness score 30 minutes after taking a first dose of the primary therapeutic agent, and, optionally, the first dose of the treatment that increases tear production, and at a later point, the subject's score is the same or lesser (indicating a benefit to the subject), then the statistically significant improvement is said to be maintained. Alternatively, if at a later point, the subject's Eye Dryness score is greater than the Eye Dryness score 30 minutes after taking a first dose of the primary therapeutic agent, and, optionally, the first dose of the treatment that increases tear production, the subject may still be receiving a therapeutic benefit and the later determined score may still be a statistically significant improvement compared to prior administration of the first dose, or their pre-treatment baseline score.

In another example, if there is a statistically significant decrease (improvement) in a subject's Eye Dryness score 1 week after taking a first dose of the primary therapeutic agent, and, optionally, the first dose of the treatment that increases tear production, and at a later point, the subject's score is the same or lesser (indicating a benefit to the subject), then the statistically significant improvement is said to be maintained. Alternatively, if at a later point, the subject's Eye Dryness score is greater than the Eye Dryness score 1 week after taking a first dose of the primary therapeutic agent, and, optionally, the first dose of the treatment that increases tear production, the subject may still be receiving a therapeutic benefit and the later determined score may still be a statistically significant improvement compared to prior administration of the first dose, or their pre-treatment baseline score.

Note that what constitutes an improvement differs depending on the score being measured. For example, an improvement in an Eye Dryness score, the corneal score and the OSDI score is a decrease in the numerical value of the score. For the Schirmer's test, an improvement is typically an increase in the numerical value of the Schirmer's score.

In some of the embodiments described herein, the maintenance of the statistically significant improvement of the subject's score (e.g., EDS, Schirmer, corneal, or OSDI) means that the statistically significant improvement does not diminish by more than 10%, 20%, 30%, 40%, 50%, or 60%.

In some of the embodiments described herein, the statistically significant improvement in the subject's score (e.g., EDS, Schirmer, corneal, or OSDI) is maintained for at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months. In some of the embodiments described herein, the statistically significant improvement in the subject's score is maintained for at least 30 minutes from administration of the first dose of the primary therapeutic agent, and, optionally, the first dose of the treatment that increases tear production, wherein the statistically significant improvement does not diminish by more than 30%. In some embodiments, the statistically significant improvement in the subject's score is maintained for at least 1 week from administration of the first dose of the primary therapeutic agent, and, optionally, the first dose of the treatment that increases tear production, wherein the statistically significant improvement does not diminish by more than 30%.

In some of the embodiments described herein, the statistically significant improvement in the subject's score (e.g., EDS, Schirmer, corneal, or OSDI) is maintained for at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the primary therapeutic agent, and, optionally, the first dose of the treatment that increases tear production, wherein the statistically significant improvement does not diminish by more than 10%, 20%, 30%, 40%, 50%, or 60% compared to the subject's score within 5 minutes after administering the first dose of the primary therapeutic agent, and, optionally, the first dose of the treatment that increases tear production.

In some of the embodiments described herein, the statistically significant improvement in the subject's score (e.g., EDS, Schirmer, corneal, or OSDI) is maintained for at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the primary therapeutic agent, and, optionally, the first dose of the treatment that increases tear production, wherein the statistically significant improvement does not diminish by more than 20% compared to the subject's corresponding Eye Dryness Score, corneal score, or OSDI score within 5 minutes after administering the first dose of the primary therapeutic agent, and, optionally, the first dose of the treatment that increases tear production.

In some of the embodiments described herein, the statistically significant improvement in the subject's score (e.g., EDS, Schirmer, corneal, or OSDI) is maintained for at least 30 minutes after administering the first dose of the primary therapeutic agent, and, optionally, the first dose of the treatment that increases tear production, wherein the statistically significant improvement does not diminish by more than 10%, 20%, or 30% compared to the subject's score within 5 minutes after administering the first dose of the primary therapeutic agent, and, optionally, the first dose of the treatment that increases tear production.

In some of the embodiments described herein, the statistically significant improvement in the subject's score (e.g., EDS, Schirmer, corneal, or OSDI) is maintained for at least 4 weeks after administering the first dose of the primary therapeutic agent, and, optionally, the first dose of the treatment that increases tear production, wherein the statistically significant improvement does not diminish by more than 10%, 20%, or 30% compared to the subject's score within 1 week after administering the first dose of the primary therapeutic agent, and, optionally, the first dose of the treatment that increases tear production.

In some of the embodiments described herein, the subject's Schirmer's score at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the primary therapeutic agent, and, optionally, the first dose of the treatment that increases tear production, does not decrease by more than 10%, 20%, 30%, 40%, 50%, or 60% compared to the subject's corresponding Schirmer's score within 5 minutes after administering the first dose of the primary therapeutic agent, and, optionally, the first dose of the treatment that increases tear production.

In some of the embodiments described herein, the subject's Schirmer's score at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the primary therapeutic agent, and, optionally, the first dose of the treatment that increases tear production, does not decrease by more than 30% compared to the subject's corresponding Schirmer's score within 5 minutes after administering the first dose of the primary therapeutic agent, and, optionally, the first dose of the treatment that increases tear production.

In some of the embodiments described herein, the subject's Schirmer's score at least 30 minutes after administering the first dose of the primary therapeutic agent, and, optionally, the first dose of the treatment that increases tear production, does not decrease by more than 30%, 20%, or 10% compared to the subject's corresponding Schirmer's score within 5 minutes after administering the first dose of the primary therapeutic agent, and, optionally, the first dose of the treatment that increases tear production.

In some of the embodiments described herein, the subject's Schirmer's score at least 4 weeks after administering the first dose of the primary therapeutic agent, and, optionally, the first dose of the treatment that increases tear production, does not decrease by more than 30%, 20%, or 10% compared to the subject's corresponding Schirmer's score within 1 week after administering the first dose of the primary therapeutic agent, and, optionally, the first dose of the treatment that increases tear production.

In some of the embodiments described herein, the subject's Eye Dryness score, corneal score, or OSDI score at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the primary therapeutic agent, and, optionally, the first dose of the treatment that increases tear production, does not decrease by more than 10%, 20%, 30%, 40%, 50%, or 60% compared to the subject's corresponding Eye Dryness score, corneal score, or OSDI score within 5 minutes after administering the first dose of the primary therapeutic agent, and, optionally, the first dose of the treatment that increases tear production.

In some of the embodiments described herein, the subject's Eye Dryness score, corneal score, or OSDI score at least 30 minutes, at least 45 minutes, at least 60 minutes, at least 2 hours, at least 4 hours, at least 6 hours, at least 8 hours, at least 1 day, at least 1 week, at least 1 month, at least 3 months, at least 6 months, at least 9 months, or at least 12 months after administering the first dose of the primary therapeutic agent, and, optionally, the first dose of the treatment that increases tear production, does not decrease by more than 30% compared to the subject's corresponding Eye Dryness score, corneal score, or OSDI score within 5 minutes after administering first dose of the primary therapeutic agent, and, optionally, the first dose of the treatment that increases tear production.

In some of the embodiments described herein, the subject's Eye Dryness score, corneal score, or OSDI score at least 30 minutes after administering first dose of the primary therapeutic agent, and, optionally, the first dose of the treatment that increases tear production, does decrease by more than 30%, 20%, or 10% compared to the subject's corresponding Eye Dryness score, corneal score, or OSDI score within 5 minutes after administering the first dose of the primary therapeutic agent, and, optionally, the first dose of the treatment that increases tear production.

In some of the embodiments described herein, the subject's Eye Dryness score, corneal score, or OSDI score at least 4 weeks after administering first dose of the primary therapeutic agent, and, optionally, the first dose of the treatment that increases tear production, does not decrease by more than 30%, 20%, or 10% compared to the subject's corresponding Eye Dryness score, corneal score, or OSDI score within 1 week after administering the first dose of the primary therapeutic agent, and, optionally, the first dose of the treatment that increases tear production.

### Humidity

The term “humidity” may refer to relative humidity, which is the ratio of the partial pressure of water vapor to the equilibrium vapor pressure of water at a given temperature. Relative humidity depends on temperature and the pressure of the system of interest. It requires less water vapor to attain high relative humidity at low temperatures; more water vapor is required to attain high relative humidity in warm or hot air. In some of the embodiments described herein, the subject is present in an environment with reduced relative humidity between determinations of the subject's score (Eye dryness, Schirmer's, corneal, OSDI).

Between determinations of a subject's scoring on a particular test, the subject may be present or exposed to an environment artificially created to adversely challenge the subject. For instance, the subject may be in a room where the temperature, humidity, air flow is monitored and controlled to create adverse conditions. In another example, the subject may wear desiccating googles and their eyes may be challenged by low relative humidity.

In some of the embodiments described herein, between determinations of the subject's Eye Dryness Score, between determinations of the subject's Schirmer's score, between determinations of the subject's corneal score, and between determinations of the subject's OSDI score, the subject is present in an environment with reduced humidity. In some of the embodiments described herein, between determinations of the subject's Eye Dryness Score, between determinations of the subject's Schirmer's score, between determinations of the subject's corneal score, and between determinations of the subject's OSDI score, the subject is present in an environment with reduced humidity and a temperature lower or higher than room temperature. In some of the embodiments described herein, the reduced relative humidity at room temperature is less than 40%, less than 30%, less than 20%, less than 10%, or less than 5%. Room temperature is between 15 degrees Celsius (59 degrees Fahrenheit) and 25 degrees Celsius (77 degrees Fahrenheit).

### Dosing Timing and Method of Administration

The schedule of doses administered to a subject depends on various considerations including the duration of effectiveness of each dose, the pharmacokinetic profile of the drug, and the effect of the dose on the body. For example, wherein the patient's condition does not improve, upon the health provider's discretion, the administration of the primary therapeutic agent, or the treatment that increases tear production, may be increased in frequency or administered chronically in order to ameliorate or otherwise control or limit the symptoms of the subject's disease or condition. In another example, if a subject's condition does improve, upon the health provider's discretion, the frequency of administration of a dose of the primary therapeutic agent, or a dose of the treatment that increases tear production, may be reduced while maintaining effective treatment of the subject. For instance, the period of time between administrations of one or more doses is extended, or the period of time between days the subject is administered one or more doses is extended. As a non-limiting example, daily administration of one or more doses is modified to administration of one or more doses every other day, or once a week.

The term “dose”, as used herein, may refer to a dose of the primary therapeutic agent, or a dose of the treatment that increases tear production.

In some of the embodiments described herein, a dose of the primary therapeutic agent is a dose of a viral vector carrying a polynucleotide encoding a gene product. In such cases, delivery of an appropriate dose (e.g., effective amount) of the gene product is achieved by administering an appropriate amount/titer of the viral vector to the target site which allows expression of a therapeutically effective amount of the gene product over a period of time. In some embodiments, the viral vector is an AAV vector. In some embodiments, the viral vector is administered to the lacrimal gland. In some embodiments, the viral vector is administered to the lacrimal gland by topical administration. In some embodiments, the viral vector is administered to the lacrimal gland by direct injection. In some embodiments, a dose of the viral vector results in the stable production of the gene product for a period of time (e.g, about 1 day, about 2 days, about 4 days, about 1 week, about 2 weeks, about 3 weeks, about 4 weeks, about 1 month, about 2 months, about 3 months, about 4 months, about 5 months, about 6 months, about 9 months, about 12 months, or longer).

In some embodiments, a dose of the viral vector results in the stable production of the gene product for about 1 week. In some embodiments, a dose of the viral vector results in the stable production of the gene product for about 2 weeks. In some embodiments, a dose of the viral vector results in the stable production of the gene product for about 3 weeks. In some embodiments, a dose of the viral vector results in the stable production of the gene product for about 4 weeks. In some embodiments, a dose of the viral vector results in the stable production of the gene product for about 1 month. In some embodiments, a dose of the viral vector results in the stable production of the gene product for about 2 months. In some embodiments, a dose of the viral vector results in the stable production of the gene product for about 3 months. In some embodiments, a dose of the viral vector results in the stable production of the gene product for about 4 months. In some embodiments, a dose of the viral vector results in the stable production of the gene product for about 5 months. In some embodiments, a dose of the viral vector results in the stable production of the gene product for about 6 months. In some embodiments, a dose of the viral vector results in the stable production of the gene product for about 9 months. In some embodiments, a dose of the viral vector results in the stable production of the gene product for about 12 months.

In some embodiments, the method comprises delivering a therapeutically effective amount of a gene product by administering a viral vector carrying a polynucleotide encoding the gene product. In some embodiments, the method comprises delivering an effective amount of a gene product by administering a viral vector carrying a polynucleotide encoding the gene product. In some embodiments, the method comprises delivering a first dose and one or more subsequent doses of the primary therapeutic agent. The one or more subsequent doses are administered after a period of time after the first dose. In some embodiments, this period of time between the first dose and the next subsequent dose is at least 1 day, at least 3 days, at least 1 week, at least 2 weeks, at least 3 weeks, at least 4 weeks, at least 1 month, at least 2 months, at least 4 months, at least 6 months, at least 9 months, at least 12 months, or longer. In some embodiments, this period of time between the first dose and the next subsequent dose is between 1-7 days, between 1-4 weeks, between 2-6 weeks, between 4-8 weeks, between 1-3 months, between 2-4 months, between 3-6 months, between 4-12 months, between 6-24 months. In some embodiments, the period of time between the one or more subsequent doses is at least 1 day, at least 3 days, at least 1 week, at least 2 weeks, at least 3 weeks, at least 4 weeks, at least 1 month, at least 2 months, at least 4 months, at least 6 months, at least 9 months, at least 12 months, or longer. In some embodiments, the period of time between the one or more subsequent doses is between 1-7 days, between 1-4 weeks, between 2-6 weeks, between 4-8 weeks, between 1-3 months, between 2-4 months, between 3-6 months, between 4-12 months, between 6-24 months.

In some embodiments, the primary therapeutic agent is administered in repeat doses. In some embodiments, the primary therapeutic agent is administered in 1, 2, 3, 4, 5, 6, 7, 8, 9, 10, or more doses. In some embodiments, the primary therapeutic agent is administered in two doses. In some embodiments, the primary therapeutic agent is administered in three doses. In some embodiments, the primary therapeutic agent is administered in four doses. In some embodiments, the primary therapeutic agent is administered in five doses. In some embodiments, the primary therapeutic agent is administered in six doses. In some embodiments, the primary therapeutic agent is administered in seven doses. In some embodiments, the primary therapeutic agent is administered in eight doses. In some embodiments, the primary therapeutic agent is administered in nine doses. In some embodiments, the primary therapeutic agent is administered in ten doses.

In some embodiments, the doses are the same concentration of primary therapeutic agent. In some embodiments, the doses are different concentrations of primary therapeutic agent. In some embodiments, the repeat doses are administered to the same eye or lacrimal gland. In some embodiments, the repeat doses are administered to at least one eye or lacrimal gland.

In some embodiments, the treatment that increases tear production is administered on a separate schedule from the administration of primary therapeutic agent. In some embodiments, the treatment that increases tear production is administered throughout the period between the multiple doses of the primary therapeutic agent. In some embodiments, the treatment that increases tear production is administered after the first dose of the primary therapeutic agent. In some of the embodiments described herein, the treatment that increases tear production is administered for at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least one year, or longer. In some of the embodiments described herein, the treatment that increases tear production is administered for 2-52 weeks, 2-40 weeks, 2-36 weeks, 2-24 weeks, 2-12 weeks, 2-8 weeks, 4-52 weeks, 4-40 weeks, 4-36 weeks, 4-24 weeks, 4-12 weeks, 4-8 weeks, 5-52 weeks, 5-40 weeks, 5-36 weeks, 5-24 weeks, 5-12 weeks, 5-8 weeks, 6-52 weeks, 6-40 weeks, 6-36 weeks, 6-24 weeks, 6-12 weeks, or 6-8 weeks.

In some embodiments, the method comprises administering a treatment that increases tear production. In some embodiments, the method comprises delivering an effective amount treatment that increases tear production. In some embodiments, the method comprises delivering a first dose and one or more subsequent doses of the treatment that increases tear production. The one or more subsequent doses are administered after a period of time after the first dose. In some embodiments, this period of time between the first dose and the next subsequent dose is at least 1 day, at least 3 days, at least 1 week, at least 2 weeks, at least 3 weeks, at least 4 weeks, at least 1 month, at least 2 months, at least 4 months, at least 6 months, at least 9 months, at least 12 months, or longer. In some embodiments, this period of time between the first dose and the next subsequent dose is between 1-7 days, between 1-4 weeks, between 2-6 weeks, between 4-8 weeks, between 1-3 months, between 2-4 months, between 3-6 months, between 4-12 months, between 6-24 months. In some embodiments, the period of time between the one or more subsequent doses is at least 1 day, at least 3 days, at least 1 week, at least 2 weeks, at least 3 weeks, at least 4 weeks, at least 1 month, at least 2 months, at least 4 months, at least 6 months, at least 9 months, at least 12 months, or longer. In some embodiments, the period of time between the one or more subsequent doses is between 1-7 days, between 1-4 weeks, between 2-6 weeks, between 4-8 weeks, between 1-3 months, between 2-4 months, between 3-6 months, between 4-12 months, between 6-24 months.

In some of the embodiments described herein, a dose of the primary therapeutic agent and/or treatment that increases tear production is administered to the subject in need thereof one to four times daily after the first day of administration of the primary therapeutic agent, once a day after the first day of administration of the primary therapeutic agent, twice a day after the first day of administration of the primary therapeutic agent, or three times a day after the first day of administration of the primary therapeutic agent.

In some of the embodiments described herein, the length of time between dosing is increased. For instance, administration of a dose every 4 hours is modified to administration of a dose every 8 or 12 hours.

In some of the embodiments described herein, the total amount of the primary therapeutic agent, or the total amount of the treatment that increases tear production, per dose is reduced over time. For instance, a dose of 2000 micrograms is reduced to 1000 micrograms, wherein administration of reduced dose maintains the effective treatment of the subject. In some embodiments described herein, the reduced dosage is provided by a reduction in administrations of the primary therapeutic agent, or the treatment that increases tear production. For instance, where administered as a nasal spray, a dose comprising two administrations or sprays of a treatment that increases tear production is reduced by administering only one spray.

In some of the embodiments described herein, the dose comprises multiple administrations of the treatment that increases tear production to each nostril. In some of the embodiments described herein, the dose comprises multiple administrations of the treatment that increases tear production to one nostril.

In some of the embodiments described herein, the dose comprises a single administration of the treatment that increases tear production to each nostril. In some of the embodiments described herein, the dose comprises a single administration of the treatment that increases tear production, to one nostril.

In some of the embodiments described herein, the treatment that increases tear production is administered to one nostril per dose. In some of the embodiments described herein, the treatment that increases tear production is administered to both nostrils per dose.

In some of the embodiments described herein, the primary therapeutic agent, or the treatment that increases tear production, is administered for at least 28 days. In some of the embodiments described herein, the primary therapeutic agent is administered one or more times concurrently with or at intervals with the treatment that increases tear production. Administration of the treatment that increases tear production can then be continued. In some of the embodiments described herein, the treatment that increases tear production is administered for at least one or more months.

In some of the embodiments described herein, the treatment that increases tear production is administered for at least 2 months, at least 3 months, at least 4 months, at least 5 months, at least 6 months, at least 7 months, at least 8 months, at least 9 months, at least 10 months, at least 11 months, or at least one year. In some of the embodiments described herein, the treatment that increases tear production is administered for 2-52 weeks, 2-40 weeks, 2-36 weeks, 2-24 weeks, 2-12 weeks, 2-8 weeks, 4-52 weeks, 4-40 weeks, 4-36 weeks, 4-24 weeks, 4-12 weeks, 4-8 weeks, 5-52 weeks, 5-40 weeks, 5-36 weeks, 5-24 weeks, 5-12 weeks, 5-8 weeks, 6-52 weeks, 6-40 weeks, 6-36 weeks, 6-24 weeks, 6-12 weeks, or 6-8 weeks.

In some of the embodiments described herein, the method comprises a first dose and one or more subsequent doses of the effective amount of the primary therapeutic agent. The one or more subsequent doses are administered after a period of time after the first dose. This period of time between the first dose and the next subsequent dose is at least 1 hour, at least 2 hours, at least 3 hours, at least 4 hours, at least 5 hours, at least 6 hours, at least 7 hours, or at least 8 hours. The period of time between the first dose and the next subsequent dose is between 1-3 hours, 2-4 hours, 3-6 hours, or 4-8 hours. The period of time between the one or more subsequent doses is at least 1 hour, at least 2 hours, at least 3 hours, at least 4 hours, at least 5 hours, at least 6 hours, at least 7 hours, or at least 8 hours. The period of time between the one or more subsequent doses is between 1-3 hours, 2-4 hours, 3-6 hours, or 4-8 hours.

In some embodiments described herein, the treatment that increases tear production, is administered to the subject in need thereof after the primary therapeutic agent. In some embodiments described herein, the treatment that increases tear production, is administered to the subject in need thereof before the primary therapeutic agent. In some embodiments described herein, the treatment that increases tear production, and the primary therapeutic agent, are administered to the subject in need thereof at the same time.

In some of the embodiments described herein, the method comprises administering a dose of an effective amount of a treatment that increases tear production, and a dose of an effective amount of a primary therapeutic agent. In some embodiments, for each dose of primary therapeutic agent administered, a dose of the treatment that increases tear production is administered before the primary therapeutic agent. In some embodiments, for each dose of the primary therapeutic agent administered, a dose of the treatment that increases tear production is administered after the primary therapeutic agent. In some embodiments, for each dose of the primary therapeutic agent, a dose of the treatment that increases tear production is administered at the same time as the primary therapeutic agent. In some embodiments, for each dose of the primary therapeutic agent, multiple doses of the treatment that increases tear production are administered over time after administration of the primary therapeutic agent.

Over the course of chronic treatment, wherein the subject is administered multiple doses of either or both of the primary therapeutic agent and the treatment that increases tear production over a period of time, the subject may be administered more doses of the primary therapeutic agent than doses of the treatment that increases tear production, or vice versa. The ratio of a dose of a primary therapeutic agent and a dose of a treatment that increases tear production administered to a subject is not required to be 1:1. The ratio will depend on the dosing regimen prescribed to the subject. In some of the embodiments disclosed herein, the ratio of a dose of a primary therapeutic agent and a dose of a treatment that increases tear production administered to a subject is 1:1, 1:2, 1:3, 1:4, 1:5, 1:6, 1:7, 1:8, 1:9, or 1:10 over a treatment course.

In some of the embodiments described herein, the method comprises administering a first dose, and one or more subsequent doses, of an effective amount of a primary therapeutic agent, and a first dose, and one or more subsequent doses, of an effective amount of a primary therapeutic agent.

In some embodiments described herein, the time period between administration of a dose of a primary therapeutic agent, and a dose of an effective amount of a treatment that increases tear production is less than 5 minutes, between 5-60 minutes, between 30-90 minutes, between 1-3 hours, between 1-8 hours, between 1-12 hours, between 1-24 hours, between 8-12 hours, between 8-24 hours, between 12-24 hours, between 1-3 days, between 1-7 days, between 1-14 days, between 1-28 days, between 3-7 days, between 3-14 days, between 3-28 days, between 7-14 days, or between 7-28 days.

In some of the embodiments described here, the method comprises administering a first dose and one or more subsequent doses of the primary therapeutic agent, and a first dose and one or more subsequent doses of the treatment that increases tear production. The one or more subsequent doses are administered after a period of time after the first dose. This period of time between the first dose and the next subsequent dose is at least 1 hour, at least 2 hours, at least 3 hours, at least 4 hours, at least 5 hours, at least 6 hours, at least 7 hours, or at least 8 hours. The period of time between the first dose and the next subsequent dose is between 1-3 hours, 2-4 hours, 3-6 hours, or 4-8 hours. The period of time between the one or more subsequent doses is at least 1 hour, at least 2 hours, at least 3 hours, at least 4 hours, at least 5 hours, at least 6 hours, at least 7 hours, or at least 8 hours. The period of time between the one or more subsequent doses is between 1-3 hours, 2-4 hours, 3-6 hours, or 4-8 hours.

In some of the embodiments described herein, the treatment that increases tear production, and the primary therapeutic agent, are administered to the subject in need thereof in separate dosage forms. In some of the embodiments described herein, the treatment that increases tear production, and the primary therapeutic agent, are administered to the subject in need thereof in a combined dosage form.

In some embodiments, the treatment that increases tear production is administered subsequence to administration of the primary therapeutic agent. In some embodiments, the treatment that increases tear production is administered beginning 4, 5, 6, 7, 8, 9, or 10 days subsequent to administration of the primary therapeutic agent. In some embodiments, the treatment that increases tear production is administered beginning 4 days subsequent to administration of the primary therapeutic agent. In some embodiments, the treatment that increases tear production is administered beginning 5 days subsequent to administration of the primary therapeutic agent. In some embodiments, the treatment that increases tear production is administered beginning 6 days subsequent to administration of the primary therapeutic agent. In some embodiments, the treatment that increases tear production is administered beginning 7 days subsequent to administration of the primary therapeutic agent. In some embodiments, the treatment that increases tear production is administered beginning 8 days subsequent to administration of the primary therapeutic agent. In some embodiments, the treatment that increases tear production is administered beginning 9 days subsequent to administration of the primary therapeutic agent. In some embodiments, the treatment that increases tear production is administered beginning 10 days subsequent to administration of the primary therapeutic agent.

### Dosage Amount and Volumes of Tear Stimulator

In some of the embodiments described herein, the dosages described herein are administered for prophylactic and/or therapeutic treatments. Dosage amounts effective for therapeutic treatments depends on the severity and course of the disease or condition, previous therapy, the patient's health, status, weight, and response to the drugs, and the judgment of the treating health provider. Therapeutically effective amounts are optionally determined by methods including, but not limited to, a dose escalation clinical trial.

In prophylactic applications, the pharmaceutical formulations described herein are administered to a patient susceptible to or otherwise at risk of a particular disease, disorder or condition. Such an amount is defined to be a “prophylactically effective amount or dose.” In this use, the amount will depend on the severity and course of the disease, disorder, or condition, previously therapy, the patient's health status and response to the drugs, and the judgment of the treating health provider.

In some of the embodiments described herein, the amount per dose of the nAChR agonist administered to the subject is 5-4000 micrograms, 5-1000 micrograms, 10-2000 micrograms, 10-700 micrograms, 100-700 micrograms, 100-600 micrograms, 100-500 micrograms, 200-700 micrograms, 200-600 micrograms, 200-500 micrograms, 300-600 micrograms, 300-500 micrograms, 900-4000 micrograms, 900-3000 micrograms, 900-2500 micrograms, 900-2000 micrograms, 1000-4000 micrograms, 1000-2500 micrograms, 1000-2000 micrograms, 1250-4000 micrograms, 1250-2500 micrograms, 1250-2000 micrograms, 1500-4000 micrograms, 1500-3000 micrograms, 1500-2500 micrograms, 1500-2000 micrograms, 1800-4000 micrograms, 1800-3000 micrograms, 1800-2500 micrograms, 1800-2250 micrograms, 2000-4000 micrograms, 2000-3000 micrograms, or 2000-2500 micrograms, or a corresponding amount of a pharmaceutically acceptable salt thereof.

In some of the embodiments described herein, the amount per dose of the nAChR agonist administered to the subject is 900-2500 micrograms, 1000-2500 micrograms, 1500-3000 micrograms, 1800-2500 micrograms, 1800-2250 micrograms, or a corresponding amount of a pharmaceutically acceptable salt thereof. In some of the embodiments described herein, the amount per dose of varenicline or compound 1 administered to the subject is 900-2500 micrograms, 1000-2500 micrograms, 1500-3000 micrograms, 1800-2500 micrograms, 1800-2250 micrograms, or a corresponding amount of a pharmaceutically acceptable salt thereof.

In some of the embodiments described herein, the amount per dose of varenicline administered to the subject is 5-15 micrograms, 50-65 micrograms, 100-125 micrograms, or a corresponding amount of a pharmaceutically acceptable salt thereof.

In some of the embodiments described herein, the amount per dose of compound 1 administered to the subject is 150-300 micrograms, 900-1200 micrograms, 2100-2400 micrograms, or a corresponding amount of a pharmaceutically acceptable salt thereof.

In some of the embodiments described herein, the pharmaceutically acceptable salt of the nAChR agonist is administered. In some of the embodiments described herein, the free base of the nAChR agonist is administered.

The volume of the pharmaceutical formulation administered to a subject depends on various factors, including the route of administration and the type of delivery device. For nasal administration, the volume of the pharmaceutical formulation should be sufficient to deliver the effective amount of the drug to the nasal cavity. Too little a volume might result in the drug not reaching the nasal cavity. On the other hand, the volume should not be so large as to be impractical, uncomfortable, or too difficult to administer to the subject. In addition, too large of a volume may result in the pharmaceutical formulation being delivered to areas of the body not intended for delivery. This can result in waste of the pharmaceutical formulation or irritation of tissues. For instance, reducing a dose volume from 200 microliters to 100 microliters may reduce the incidence of irritation of the upper throat/soft palate by reducing post-nasal drip after instillation.

In some of the embodiments described herein, the nAChR agonist, or a pharmaceutically acceptable salt thereof, is administered in a pharmaceutical formulation for nasal administration, and the total volume of the pharmaceutical formulation per dose is 50-250 microliters, 75-125 microliters, 150-250 microliters, or 175-225 microliters.

In some of the embodiments described herein, the nAChR agonist, or a pharmaceutically acceptable salt thereof, is administered in a pharmaceutical formulation for nasal administration, and the total volume of the pharmaceutical formulation per dose is about 50 microliters, about 75 microliters, about 100 microliters, about 125 microliters, about 150 microliters, about 175 microliters, about 200 microliters, about 225 microliters, or about 250 microliters.

In some of the embodiments described herein, the nAChR agonist, or a pharmaceutically acceptable salt thereof, is administered in a pharmaceutical formulation for nasal administration, and the total volume of the pharmaceutical formulation per nostril is 50-250 microliters, 75-125 microliters, 150-250 microliters, or 175-225 microliters.

In some of the embodiments described herein, the nAChR agonist, or a pharmaceutically acceptable salt thereof, is administered in a pharmaceutical formulation for nasal administration, and the total volume of the pharmaceutical formulation per nostril is about 50 microliters, about 75 microliters, about 100 microliters, about 125 microliters, about 150 microliters, about 175 microliters, about 200 microliters, about 225 microliters, or about 250 microliters.

In some of the embodiments described herein, the nAChR agonist, or a pharmaceutically acceptable salt thereof, is administered in a pharmaceutical formulation for nasal administration comprising between 1 mg/mL and 40 mg/mL, between 1 mg/mL and 30 mg/mL, between 1 mg/mL and 20 mg/mL, between 1 mg/mL and 10 mg/mL, between 1 mg/mL and 5 mg/mL, 2 mg/mL and 40 mg/mL, between 2 mg/mL and 30 mg/mL, between 2 mg/mL and 20 mg/mL, between 2 mg/mL and 10 mg/mL, between 2 mg/mL and 5 mg/mL, 5 mg/mL and 40 mg/mL, between 5 mg/mL and 30 mg/mL, between 5 mg/mL and 20 mg/mL, between 5 mg/mL and 10 mg/mL, or between 5 mg/mL and 15 mg/mL, of the nAChR agonist, or a corresponding amount of a pharmaceutically acceptable salt thereof.

In some of the embodiments described herein, the nAChR agonist, or a pharmaceutically acceptable salt thereof is administered in a pharmaceutical formulation for nasal administration, wherein the concentration of the nAChR agonist per dose is 0.01% (w/v) to 0.5% (w/v), 0.01% (w/v) to 1.0% (w/v), 0.01% (w/v) to 1.5% (w/v), 0.01% (w/v) to 2.0% (w/v), 0.01% (w/v) to 2.5% (w/v), 0.01% (w/v) to 3.0% (w/v), 0.5% (w/v) to 1.0% (w/v), 0.5% (w/v) to 1.5% (w/v), 0.5% (w/v) to 2.0% (w/v), 0.5% (w/v) to 2.5% (w/v), 0.5% (w/v) to 3.0% (w/v), 1.0% (w/v) to 1.5% (w/v), 1.0% (w/v) to 2.0% (w/v), 1.0% (w/v) to 2.5% (w/v), 1.0% (w/v) to 3.0% (w/v), 1.5% (w/v) to 2.0% (w/v), 1.5% (w/v) to 2.5% (w/v), 1.5% (w/v) to 3.0% (w/v), 2.0% (w/v) to 2.5% (w/v), or 2.0% (w/v) to 3.0% (w/v), or a corresponding amount of a pharmaceutically acceptable salt thereof.

In some of the embodiments described herein, varenicline, or a pharmaceutically acceptable salt thereof is administered in a pharmaceutical formulation for nasal administration, wherein the concentration of varenicline per dose is about 0.058% (w/v), or about 0.12% (w/v), or a corresponding amount of a pharmaceutically acceptable salt thereof.

In some of the embodiments described herein, varenicline, or a pharmaceutically acceptable salt thereof, is administered in a pharmaceutical formulation for nasal administration, wherein the concentration of varenicline, per dose is less than about 0.06% (w/v), or less than 0.15% (w/v), or a corresponding amount of a pharmaceutically acceptable salt thereof.

In some of the embodiments described herein, varenicline, or a pharmaceutically acceptable salt thereof, is administered in a pharmaceutical formulation for nasal administration, wherein the concentration of varenicline, per dose is about 0.058% (w/v), or about 0.12% (w/v), or a corresponding amount of a pharmaceutically acceptable salt thereof; and wherein the volume per dose is about 50 microliters.

In some of the embodiments described herein, varenicline, or a pharmaceutically acceptable salt thereof, is administered in a pharmaceutical formulation for nasal administration, wherein the concentration of varenicline, per dose is less than about 0.06% (w/v), or less than 0.15% (w/v), or a corresponding amount of a pharmaceutically acceptable salt thereof; and wherein the volume per dose is about 50 microliters.

In some of the embodiments described herein, varenicline, or a pharmaceutically acceptable salt thereof, is administered in a pharmaceutical formulation for nasal administration, wherein the concentration of varenicline, per dose is about 0.058% (w/v), or about 0.12% (w/v), or a corresponding amount of a pharmaceutically acceptable salt thereof; and wherein the volume per dose is about 100 microliters. In some of the embodiments herein, the 100 microliter dose is delivered as two 50 microliter sprays. In some of the embodiments herein, the 100 microliter dose is delivered as a single 100 microliter spray.

In some of the embodiments described herein, varenicline, or a pharmaceutically acceptable salt thereof, is administered in a pharmaceutical formulation for nasal administration, wherein the concentration of varenicline, per dose is less than about 0.06% (w/v), or less than 0.15% (w/v), or a corresponding amount of a pharmaceutically acceptable salt thereof; and wherein the volume per dose is about 100 microliters. In some of the embodiments herein, the 100 microliter dose is delivered as two 50 microliter sprays. In some of the embodiments herein, the 100 microliter dose is delivered as a single 100 microliter spray.

In some of the embodiments described herein, the nAChR agonist is a pharmaceutically acceptable salt. In some of the embodiments described herein, the nAChR is compound 1 galactarate (e.g., hemi-galactarate dihydrate) or compound 1 citrate (e.g., a mono-citrate salt). In some of the embodiments described herein, compound 1 is a free base. In some of the embodiments described herein, the nAChR agonist is varenicline tartrate.

The amount of nAChR agonist free base and the corresponding amount of salt administered to a subject in need thereof may be calculated based on the nAChR agonist concentration of the pharmaceutical formulation and volume of the pharmaceutical formulation administered to the subject need thereof. The following examples illustrate the relationship between concentration and volume of the pharmaceutical formulation, and the amounts of nAChR agonist salt and free base administered to the subject.

For example, a 2.0% compound 1 hemi-galactarate dihydrate solution is equivalent to 20 mg of the salt per 1 mL of solution. The 2.0% compound 1 hemi-galactarate dihydrate solution corresponds to a 1.1% compound 1 free base solution. 1.1% of free base solution is equivalent to 11.1 mg/mL of compound 1 free base.

50 μL of 2.0% compound 1 hemi-galactarate dihydrate solution contains about 1000 μg of compound 1 hemi-galactarate dihydrate, which is equivalent to about 554 μg of compound 1 free base. Likewise, 100 μL of 2.0% compound 1 hemi-galactarate dihydrate solution contains about 2000 μg of compound 1 hemi-galactarate dihydrate, which is equivalent to about 1108 μg of compound 1 free base. Amounts of salt and the corresponding amount of free base may be calculated for other concentrations or volumes in a similar fashion.

In another example, a 2.3% compound 1 mono-citrate solution is equivalent to 23.2 mg of the salt per 1 mL of solution. The 2.3% compound 1 mono-citrate solution corresponds to a 1.1% compound 1 free base solution. 1.1% of free base solution is equivalent to 11.1 mg/mL of compound 1 free base.

50 μL of 2.3% compound 1 mono-citrate solution contains about 1161 μg of compound 1 mono-citrate, which is equivalent to about 554 μg of compound 1 free base. Likewise, 100 μL of 2.3% compound 1 mono-citrate solution contains about 2322 μg of compound 1 mono-citrate, which is equivalent to about 1108 μg of compound 1 free base. Amounts of salt and the corresponding amount of free base may be calculated for other concentrations or volumes in a similar fashion.

In another example, a 0.10% varenicline tartrate solution is equivalent to 1.00 mg of the salt per 1 mL of solution. The 0.10% varenicline tartrate solution corresponds to 0.0585% varenicline free base in solution. 0.0585% of free base solution is equivalent to 0.584 mg/mL of varenicline free base.

50 μL of 0.10% varenicline tartrate solution contains about 50 μg of varenicline tartrate, which is equivalent to about 29.2 μg of varenicline free base. Likewise, 100 μL of 0.10% varenicline tartrate solution contains about 100 μg of varenicline tartrate, which is equivalent to about 58.5 μg of varenicline free base. Amounts of salt and the corresponding amount of free base may be calculated for other concentrations or volumes in a similar fashion.

In another example, a 0.20% varenicline tartrate solution is equivalent to 2.00 mg of the salt per 1 mL of solution. The 0.20% varenicline tartrate solution corresponds to 0.117% varenicline free base in solution. 0.117% of free base solution is equivalent to 1.17 mg/ml of varenicline free base.

50 μL of 0.20% varenicline tartrate solution contains about 100 μg of varenicline tartrate, which is equivalent to about 58.5 μg of varenicline free base. Likewise, 100 μL of 0.20% varenicline tartrate solution contains about 200 μg of varenicline tartrate, which is equivalent to about 117 μg of varenicline free base. Amounts of salt and the corresponding amount of free base may be calculated for other concentrations or volumes in a similar fashion.

The corresponding amount of a salt form may be calculated by multiplying the amount of free base by a multiplication factor. The multiplication factor is calculated by dividing the molecular weight of the salt form by the molecular weight of the free base. For instance, the multiplication factor for converting an amount of the compound of formula I free base to the mono-citrate salt is 2.096. The multiplication factor for converting an amount of the compound of formula I free base to the hemi-galactarate dihydrate is 1.805. The multiplication factor for converting an amount of varenicline free base to varenicline tartrate is 1.710.

The corresponding amount of the free base may be calculated by multiplying the amount of a salt form by a multiplication factor. The factor is calculated by dividing the molecular weight of the free base by the molecular weight of the salt form. For instance, the multiplication factor for converting an amount of varenicline tartrate to free base is 0.5846. The multiplication factor for converting an amount of the compound of formula I mono-citrate to free base is 0.477. The multiplication factor for converting an amount of the compound of formula I hemi-galactarate dihydrate to free base is 0.554.

In some of the embodiments disclosed herein, the nAChR agonist, or a pharmaceutically acceptable salt thereof, is administered to the nasal cavity of a subject. The pharmaceutical formulations described herein include, but are not limited to, liquids, suspensions, aerosols, gels, ointments, dry powders, creams, pastes, lotions, or balms.

In some of the embodiments disclosed herein, the nAChR agonist, or a pharmaceutically acceptable salt thereof, administered into the nasal cavity by a spray pump, syringe, dropper, bottle nebulizer, atomization pump, inhaler, powder spray device, vaporizer, patch, medicated stick, pipette, or jet of liquid.

In some embodiments, the nAChR agonist is TYRVAYA (varenicline solution) nasal spray, a nasal spray delivering 0.03 mg varenicline (equivalent to 0.05 mg of varenicline tartrate) in each 0.05 mL spray. TYRVAYA (varenicline solution) nasal spray is formulated for intranasal use as a clear 0.6 mg/mL strength solution.

### Side Effects

The disclosure provides methods of local administration (intranasal) of the nAChR agonist, or pharmaceutically acceptable salts thereof. Local administration has the advantage over systemic administration including reducing potential side effects by limiting the amount of drug that may cross the blood-brain barrier. In some of the embodiments disclosed herein, the nAChR agonist, or pharmaceutically acceptable salt thereof, administered is not systemically bioavailable. In some of the embodiments disclosed herein, the method does not result in undesired systemic side effects. In some of the embodiments disclosed herein, the method does not result in undesired psychoactive side effects.

In some of the embodiments described herein, the subject does not experience one or more side effects selected from the group consisting of overproduction of tears, cough, throat irritation, instillation site irritation, sneezing, nasopharyngitis, nasal irritation, toothache, dry mouth, and headache.

In some of the embodiments described herein, wherein within 5 minutes-60 minutes of administration of the first dose or one or more subsequent doses, the subject does not experience one or more side effects selected from the group consisting of overproduction of tears, cough, throat irritation, instillation site irritation, sneezing, nasopharyngitis, nasal irritation, toothache, dry mouth, and headache.

In some of the embodiments described herein, the overproduction of tears is indicated by an increase in a subject's Schirmer's score of greater than 20 mm. In some of the embodiments described herein, the overproduction of tears is indicated by excessive tearing, in an amount that would be impractical or undesirable. For example, tearing in an amount that would interfere with a subject's eye make-up, or would lead a subject to wipe away or absorb excess tears with a tissue, are both impractical and undesirable effects.

### Refractive Surgeries

In some of the embodiments described herein the subject will undergo or has undergone refractive surgery of the eye. Examples of refractive surgery include Laser-Assisted In-Situ Keratomileusis (LASIK), astigmatic keratotomy (AK), photorefractive keratectomy (PRK), and limbal relaxing incision (LRI). In some embodiments of the methods, uses and compositions for use, the refractive surgery is Laser-Assisted In-Situ Keratomileusis (LASIK). In some embodiments of the methods, uses and compositions for use, the refractive surgery is astigmatic keratotomy (AK). In some embodiments of the methods, uses and compositions for use, the refractive surgery is photorefractive keratectomy (PRK). In some embodiments of the methods, uses and compositions for use, the refractive surgery is limbal relaxing incision (LRI). In some embodiments, the compounds or compositions described herein are administered both prior to and after surgery. In some of the embodiments described herein, the subject has undergone refractive surgery of the eye within 2 weeks or is scheduled to undergo refractive surgery of the eye within 2 weeks.

### Lasik Population

In some embodiments, the primary therapeutic agent or and/or treatment that increases tear production described herein are administered prior to surgery. In some embodiments, the primary therapeutic agent or and/or treatment that increases tear production described herein are administered after surgery. In some embodiments, the primary therapeutic agent or and/or treatment that increases tear production described herein are administered both prior to and after surgery. In some of the embodiments described herein, the subject has undergone Lasik surgery within 2 weeks or is scheduled to undergo Lasik surgery within 2 weeks.

### Keratitis

Normal tear film contains a number of biologically active growth factors including nerve growth factor, epidermal growth factor, transforming growth factor-beta, hepatocyte growth factor, platelet-derived growth factor, vascular endothelial growth factor, fibroblast growth factor, keratinocyte growth factor, and insulin-like growth factor. When the cornea senses stimulation or pressure, the eyelids will close and tears will be produced to protect the cornea and the eye.

Neurotrophic keratitis causes reduced sensitivity of the cornea. Subjects with neurotrophic keratitis (NK) have impaired cornea function because the nerves that innervate the cornea cannot function properly; these nerves carry impulses that help the cornea function. Because these nerves do not function properly in NK, the outer layer of the cornea, called the epithelium, can break down, resulting in an epithelial defect. In more advanced neurotrophic keratitis, an interior layer called the cornea stroma can break down as well, resulting in thinning of the cornea. This is called stromal ‘melting’. In advanced stromal melting, the cornea can thin to a severe degree, which can result in a hole or opening to the inside of the eye, which can lead to infection and potentially loss of the eye. NK can lead to a variety of complications, including poor wound healing of the cornea, scarring of the cornea, and loss of vision. There are many different conditions that can damage the nerves serving the cornea.

A variety of therapies can be used to treat this disorder depending on how far the disorder has progressed in a subject. Most recently, recombinant human nerve growth factor (rhNGF) (Oxervate, Dompe) has been approved for the treatment of NK. Unfortunately, there are several drawbacks to this therapy, including the fact that the product needs to be administered 6 times per day at 2-hour intervals, for 8 weeks, the product is delivered with a pipette that can be cumbersome for self-administration, the product has to be refrigerated, and the cost for a 4-week supply is more than $10K.

The present disclosure provides methods, uses, and compositions for treating subjects with keratitis. In some embodiments, the keratitis is NK. In some embodiments, the methods and compositions for treating subject with NK disclosed herein stimulate natural tear film. In some embodiments, a subject with NK is administered a composition provided herein once or twice daily. In some embodiments, a subject with NK is administered a composition provided herein once or twice daily for at least 4, at least 5, at least 6, at least 7, or at least 8 weeks. In some embodiments, a subject with NK is administered a composition provided herein twice daily for at least 8 weeks. In some embodiments, a subject with NK is intranasally administered a composition provided herein twice daily for at least 8 weeks.

### Cmax Blood Plasma Concentration

The methods and uses described herein include local intranasal administration of the drug administered to increase tear production. Because the disclosed methods and uses are to local intranasal administration, the concentration of the drug administered to increase tear production (e.g., nAChR agonist, or pharmaceutically acceptable salt thereof), in the circulating blood plasma is low, compared to concentrations achieved from systemic forms of administration (e.g., ingestion of oral formulations). Low blood plasma concentrations of nAChR agonists avoids potential undesirable side effects associated with nAChR agonists in systemic circulation, such as nausea, sleep disturbance, constipation, flatulence, vomiting, dermal conditions like rash and pruritus, headaches, abdominal pain, dyspepsia, gastroesophageal reflux disease and dry mouth.

One way to characterize the blood plasma concentration of the nAChR agonist, or pharmaceutically acceptable salt thereof, in a subject is to measure the Cmax—the maximum or peak serum concentration that the nAChR agonist, or pharmaceutically acceptable salt thereof, achieves after the nAChR agonist, or pharmaceutically acceptable salt thereof, is administered to the subject and before the administration of a second dose. Methods of determining Cmax are known in the art. A non-limiting example of a protocol used to determine the pharmacokinetic profile of the compound of formula I, including determining Cmax is provided in Example 3.

In some embodiments described herein, the Cmax is calculated for a subject. In some embodiments, Cmax is calculated from the average Cmax of two or more subjects. In some embodiments, Cmax is calculated from the average Cmax of a subset population.

In some embodiments described herein, the subject in need thereof has a blood plasma Cmax of the nAChR agonist, or a pharmaceutically acceptable salt thereof, of less than 5 ng/ml, of less than 4 ng/mL, of less than 3 ng/ml, or of less than 2 ng/mL.

In some embodiments described herein, the subject in need thereof has a blood plasma Cmax of the nAChR agonist, or a pharmaceutically acceptable salt thereof, of less than 5 ng/mL, of less than 4 ng/mL, of less than 3 ng/mL, or of less than 2 ng/ml; and wherein the subject in need thereof was administered a dose comprising between 10 micrograms to 150 micrograms, 10 micrograms to 100 micrograms, 10 micrograms to 50 micrograms, 50 micrograms to 150 micrograms, 50 micrograms to 100 micrograms, 100 micrograms to 1500 micrograms, 100 micrograms to 600 micrograms, 200 micrograms to 400 micrograms, 400 micrograms to 600 micrograms, or 750 micrograms to 1200 micrograms of the nAChR agonist, or a corresponding amount of the pharmaceutically acceptable salt thereof.

In some embodiments described herein, the subject in need thereof has a blood plasma Cmax of compound 1, or a pharmaceutically acceptable salt thereof, of less than 5 ng/ml, of less than 4 ng/mL, of less than 3 ng/mL, or of less than 2 ng/ml; and wherein the subject in need thereof was administered a dose comprising between 100 micrograms to 1500 micrograms, 100 micrograms to 600 micrograms, 200 micrograms to 400 micrograms, 400 micrograms to 600 micrograms, or 750 micrograms to 1200 micrograms of compound 1, or a corresponding amount of the pharmaceutically acceptable salt thereof.

In some embodiments described herein, the subject in need thereof has a blood plasma Cmax of compound 1, or a pharmaceutically acceptable salt thereof, of less than 5 ng/ml, of less than 4 ng/ml, of less than 3 ng/mL, or of less than 2 ng/ml; and wherein the subject in need thereof was administered a dose comprising between 150 micrograms to 300 micrograms, 900 micrograms to 1200 micrograms, 2100 micrograms to 2400 micrograms of compound 1, or a corresponding amount of the pharmaceutically acceptable salt thereof.

In some embodiments described herein, the subject in need thereof has a blood plasma Cmax of varenicline, or a pharmaceutically acceptable salt thereof, of less than 5 ng/ml, of less than 4 ng/ml, of less than 3 ng/ml, or of less than 2 ng/ml; and wherein the subject in need thereof was administered a dose comprising between 5 micrograms to 15 micrograms, 50 micrograms to 65 micrograms, 100 micrograms to 125 micrograms of varenicline, or a corresponding amount of the pharmaceutically acceptable salt thereof.

Throughout the present disclosure, amounts of nAChR agonists disclosed refer to the amount of nAChR agonist free form free form present in the formulation. The term “corresponding amount” as used herein refers to the amount of a pharmaceutically acceptable salt of a nAChR agonist required to obtain the amount of nAChR free form free form recited in the formulation. It would be clear to one of skill in the art how to calculate the “corresponding amount” of the salt of a compound, such as the corresponding amount of the pharmaceutically acceptable salt of compound 1, taking into account the difference in molecular weight between the free form of a compound and a salt form. For example, 175.24 g of compound 1 free base, would correspond to 316.34 g of the hemi-galactarate dihydrate salt or 367.36 of the mono-citrate salt. In another example, 211.267 g of varenicline free base, would correspond to 361.354 of the varenicline tartrate salt.

As used herein, the term “about” is used synonymously with the term “approximately.” Illustratively, the use of the term “about” with regard to an amount indicates values slightly outside the cited values, e.g., plus or minus 0.1% to 20%, plus or minus 0.1% to 10%, plus or minus 0.1% to 5%, or plus or minus 0.1% to 2%.

Throughout the present disclosure, various embodiments discussed within the context of the methods of treating an ocular surface disorder, cornea disorder, or anterior chamber disorder or of delivering a gene product to the ocular surface may also be applied to embodiments related to compounds for use in the manufacture of a medicament, compounds for use in the various methods, pharmaceutical formulations, and kits.

Advantageously, when the primary therapeutic agent is delivered by administering a vector carrying the corresponding gene product, the method results in expression of the gene product on the ocular surface; and/or an increased amount of the gene product delivered to the ocular surface in a predetermined time (e.g., about 1 minute, about 2 minutes, about 4 minutes, about 3 minutes, about 5 minutes, about 30 minutes, about 1 hour, about 2 hours, about 4 hours, about 8 hours, about 12 hours, about 18 hours, about 1 day, about 2 days, about 3 days, about 5 days, or about 1 week) compared to administration of the viral vector alone. In some embodiments, the predetermined time is about 5 minutes. In some embodiments, the predetermined time is about 30 minutes. In some embodiments, the predetermined time is about 1 hour. In some embodiments, the predetermined time is about 2 hours. As used herein, the “predetermined time” refers to a time interval between a first reference time point (e.g., the administration of a composition or any arbitrary time point thereafter) and a second reference time point (such as collection of a biological sample). The predetermined time may be determined by theoretical considerations, such as kinetics of treatment effect or increase in tear production, or by performing a time-course experiment to determine an appropriate predetermined time for the assay used. The predetermined time is selected to enable calculation of a rate of change and/or flux of a selected molecule into the ocular surface, tear film, or another anatomical location.

In some embodiments, to measure the expression and/or amount of the gene product on the ocular surface, a collection device is used to collect biological samples from the ocular surface. In some embodiments, the collection device is used to collect sample at a predetermined time after the administration of the primary therapeutic agent. In some embodiments, the collection device is applied to the eye for a period of time (e.g., about 1 minute, about 2 minutes, about 4 minutes, about 3 minutes, about 5 minutes, about 30 minutes, or about 1 hour). In some embodiments, the collection device is applied to collect fluid from the lower cul-de-sac of the eye. In some embodiments, the collection device is applied to collect fluid from the tear meniscus. In some embodiments, the collection device is applied to collect fluid from the pre-ocular film. In some embodiments, the collection device is a wick. In some embodiments, the collection device is a Schirmer strip. In some embodiments, the collection device is a capillary tube.

In some embodiments, the amount of the gene product on the ocular surface can be measured by determining the amount of the gene product in the collected biological sample after the collection device has been applied to collect fluid from the subject. In some embodiments, protein mass-spectroscopy can be used to quantify the amount of the gene product. As a non-limiting example, the collected biological sample can be subject to protease digestion and then the amount of the gene product can be quantified via LC-MS/MS procedures. Mass-spectrometry based protein quantification methods are known in the art. In some embodiments, an amount of gene product in the collected biological sample is determined by Western blot, ELISA, quantitative PCR, Northern blot, Southern blot, dot blot, lateral flow assay, biolayer interferometry, fluorescence spectroscopy, luminescence spectroscopy, ultraviolet/visible spectroscopy, spectrophotometry, analytical ultracentrifugation centrifugation, dynamic light scattering, size-exclusion chromatograph, ion-exchange chromatograph, gel electrophoresis, capillary electrophoresis, high-performance liquid phase chromatography, reverse-phase chromatography, or other quantitative or semi-quantitative analytical method.

In some embodiments, the amount of the gene product in the collected biological sample can be determined by protein arrays. In some embodiments, the amount of the gene product in the collected biological sample can be determined by Western blotting. In some embodiments, the amount of the gene product in the collected biological sample can be determined by immunoblot assays. In some embodiments, the amount of the gene product in the collected biological sample can be determined by an electrophoresis assay (e.g., using Agilent Bioanalyzer 2100). In some embodiments, the amount of the gene product in the collected biological sample can be determined using a microfluidic based assay. In some embodiments, the amount of the gene product in the collected biological sample can be determined using a multiplex bead-based immunofluorescent sandwich assay. Non-limiting examples of assays and analyzed gene products are described in D'Souza and Tong (2014) Eye and Vision 1:6.

A statistically significant change, as used herein, to describe a subject's response to treatment may be calculated from an increase or decrease in the subject's score in a given assay. Non-limiting examples of determining whether the change in a subject's score is statistically significant and whether two sets of data are significantly different from each other include calculations based on an ANCOVA model, and statistical hypothesis tests such as t-tests and non-parametric Wilcoxon rank sum tests. Other models and statistical hypothesis tests well-known in the art are contemplated.

### Pharmaceutical Compositions and Kits

In some embodiments, any concentration of viral particles suitable to effectively transduce cells of the eye and/or lacrimal gland can be prepared for contacting cells of the eye and/or lacrimal gland in vitro or in vivo. For example, the viral particles may be formulated at a concentration of 10⁸ vector genomes per mL or more, for example, 5×10⁸ vector genomes per mL; 10⁹ vector genomes per mL; 5×10⁹ vector genomes per mL, 10¹⁰ vector genomes per mL, 5×10¹⁰ vector genomes per mL; 10¹¹ vector genomes per mL; 5×10¹¹ vector genomes per mL; 10¹² vector genomes per mL; 5×10¹² vector genomes per mL; 10¹³ vector genomes per mL; 1.5×10¹³ vector genomes per mL; 3×10¹³ vector genomes per mL; 5×10¹³ vector genomes per mL; 7.5×10¹³ vector genomes per mL; 9×10¹³ vector genomes per mL; 1×10¹⁴ vector genomes per mL, 5×10¹⁴ vector genomes per mL or more, but typically not more than 1×10¹⁵ vector genomes per mL. Similarly, any total number of viral particles suitable to provide appropriate transduction of cells of the eye and/or lacrimal gland to confer the desired effect or treat the disease can be administered to the mammal or to the primate's eye. In various embodiments, at least 10⁷; 5×10⁷; 10⁸; 5×10⁸; 10⁹; 5×10⁹, 10¹⁰, 5×10¹⁰; 10¹¹; 5×10¹¹; 10¹²; 5×10¹²; 10¹³; 1.5×10¹³; 3×10¹³; 5×10¹³; 7.5×10¹³; 9×10¹³, 1×10¹⁴ viral particles, or 5×10¹⁴ viral particles or more, but typically not more than 1×10¹⁵ viral particles are injected per eye. Any suitable number of administrations of the vector to the mammal or the primate eye can be made. In one embodiment, the methods comprise a single administration; in other embodiments, multiple administrations are made over time as deemed appropriate by an attending clinician.

The subject viral vector may be formulated into any suitable unit dosage, including, without limitation, 1×10⁸ vector genomes or more, for example, 1×10⁹, 1×10¹⁰, 1×10¹¹, 1×10¹², or 1×10¹³ vector genomes or more, in certain instances, 1×10¹⁴ vector genomes, but usually no more than 4×10¹⁵ vector genomes. In some embodiments, the viral vector is formulated into any suitable unit dosage, including, without limitation, 1×10⁷, 1×10⁸, 1×10⁹, 1×10¹⁰, 1×10¹¹, 1×10¹², or 1×10¹³ vector genomes or more. In some cases, the unit dosage is at most about 5×10¹⁵ vector genomes, e.g., 1×10¹⁴ vector genomes or less, for example 1×10¹³, 1×10¹², 1×10¹¹, 1×10¹⁰, or 1×10⁹ vector genomes or less, in certain instances 1×10⁸ vector genomes or less, and typically no less than 1×10⁸ vector genomes. In some cases, the unit dosage is at most about 5×10¹⁵ vector genomes, e.g., 1×10¹⁴ vector genomes or less, for example 1×10¹³, 1×10¹², 1×10¹¹, 1×10¹⁰, 1×10⁹, 1×10⁸, or 1×10⁷ vector genomes or less. In some cases, the unit dosage is 1×10¹⁰ to 1×10¹¹ vector genomes. In some cases, the unit dosage is 1×10¹⁰ to 3×10¹² vector genomes. In some cases, the unit dosage is 1×10⁹ to 3×10¹³ vector genomes. In some cases, the unit dosage is 1×10⁸ to 3×10¹⁴ vector genomes.

In some cases, the unit dosage of pharmaceutical composition may be measured using multiplicity of infection (MOI). By MOI it is meant the ratio, or multiple, of vector or viral genomes to the cells to which the nucleic acid may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some embodiments, the amount of pharmaceutical composition comprises about 1×10⁸ to about 1×10¹⁵ recombinant viruses, about 1×10⁹ to about 1×10¹⁴ recombinant viruses, about 1×10¹⁰ to about 1×10¹³ recombinant viruses, or about 1×10₁₁ to about 3×10¹² recombinant viruses.

In preparing the subject viral vector compositions, any host cells for producing rAAV virions may be employed, including, for example, mammalian cells (e.g., 293 cells), insect cells (e.g., SF9 cells), microorganisms and yeast. Host cells can also be packaging cells in which the AAV rep and cap genes are stably maintained in the host cell or producer cells in which the rAAV virion genome is stably maintained and packaged. Exemplary packaging and producer cells are derived from SF-9, 293, A549 or HeLa cells. rAAV virions are purified and formulated using standard techniques known in the art.

The viral vectors of the disclosure are generally delivered to the subject as a pharmaceutical composition. Pharmaceutical compositions comprise a pharmaceutically acceptable solvent (e.g. water, etc.) and one or more excipients. In some embodiments, the pharmaceutical compositions comprise a buffer at about neutral pH (pH 5, 6, 7, 8, or 9). In some embodiments, the pharmaceutical composition comprises phosphate buffered saline (e.g. PBS at pH of about 7). The pharmaceutical compositions may comprise a pharmaceutically acceptable salt. The concentration of the salt may be selected to ensure that the pharmaceutical composition is isotonic to, or nearly isotonic to, the target tissue.

In some embodiments, the disclosure provides for use of an rAAV virion described herein in the manufacture of a medicament. In some embodiments, the disclosure provides for use a treatment described herein that increases tear production. In some embodiments, the disclosure provides for use of an rAAV virion described herein in the manufacture of a medicament for use in a method described herein. In some embodiments, the disclosure provides for use of a treatment described herein that increases tear production in the manufacture of a medicament for use in a method described herein.

In some embodiments, the disclosure provides a pharmaceutical composition comprising an rAAV virion described herein. In some embodiments, the pharmaceutical composition comprises an rAAV virion described herein, and a pharmaceutically acceptable carrier, delivery agent, or excipient.

In some embodiments, the disclosure provides a pharmaceutical composition comprising a treatment described herein that increases tear production. In some embodiments, the pharmaceutical composition comprises a treatment described herein that increases tear production, and a pharmaceutically acceptable carrier, delivery agent, or excipient.

In some embodiments, the disclosure provides use of an rAAV virion or pharmaceutical composition described herein, in the manufacture of a medicament for treatment of an ocular disease, disorder, or condition. In some embodiments, the disclosure provides use of an rAAV virion or pharmaceutical composition herein for use, or adaptable for use, in the treatment of an ocular disease, disorder, or condition. In some embodiments, the disclosure provides use of a treatment described herein that increases tear production or pharmaceutical composition described herein, in the manufacture of a medicament for treatment of an ocular disease, disorder, or condition. In some embodiments, the disclosure provides use of treatment described herein that increases tear production or pharmaceutical composition herein for use, or adaptable for use, in the treatment of an ocular disease, disorder, or condition.

In some embodiments, the pharmaceutically acceptable carrier comprises phosphate buffered saline. In some embodiments, the pharmaceutical composition is formulated to be compatible with its intended route of administration (e.g. intralacrimal or nasal administration). In some embodiments, the pharmaceutical composition is formulated for administration into the lacrimal gland. In some embodiments, the pharmaceutical composition is formulated for administration onto the ocular surface. In some embodiments, the pharmaceutical composition is formulated for nasal administration.

In some embodiments, the disclosure provides a kit comprising two compositions described herein, and instructions for use. In some embodiments, the kit comprises:
(a) a first composition comprising a viral vector comprising a polynucleotide encoding a gene product, or functional variant thereof; and
(b) a second composition comprising a nicotinic acetylcholine receptor (nAChR) agonist.

In some embodiments, the disclosure provides a kit comprising:
(a) a first composition comprising a viral vector comprising a polynucleotide encoding a gene product, or functional variant thereof; and
(b) a second composition comprising a nicotinic acetylcholine receptor (nAChR) agonist, and instructions for treating or delaying progression of a disease, disorder, or condition described herein in a subject in need thereof.

In some embodiments, the kit comprises a package insert containing instructions for use of the kit. In some embodiments, the kit comprises a first composition comprising a viral vector comprising a polynucleotide encoding a gene product, and a pharmaceutically acceptable carrier, or a pharmaceutical composition comprising the first composition and instructions for treating or delaying progression of a disease, disorder, or condition described herein in a subject in need thereof.

In some embodiments, the kit comprises a second composition comprising a nicotinic acetylcholine receptor (nAChR) agonist, and a pharmaceutically acceptable carrier, or a pharmaceutical composition comprising the first composition and instructions for treating or delaying progression of a disease, disorder, or condition described herein in a subject in need thereof.

### Exemplary Embodiments

The disclosure relates to the following embodiments. Throughout this section, the term embodiment is abbreviated as “E” followed by an ordinal. For example, EI-1 is equivalent to Embodiment I-1.

Some embodiments of this disclosure relate to Embodiment I, as follows:

Embodiment I-1. A method of treating an ocular surface disorder, cornea disorder, or anterior chamber disorder in a subject, comprising:
a. administering an effective amount of a primary therapeutic agent to the eye or a tissue in fluidic communication with the eye; and
b. administering a treatment that increases tear production.

Embodiment I-2. The method of embodiment I-1, wherein the treatment that increases tear production comprises an effective amount of a nicotinic acetylcholine receptor (nAChR) agonist, or a pharmaceutically acceptable salt thereof.

Embodiment I-3. The method of embodiment I-2, wherein the nAChR agonist is a full agonist of nAChR subtypes alpha4beta2, alpha3beta4, alpha3alpha5beta4, and/or alpha4alpha6beta2.

Embodiment I-4. The method of embodiment I-2, wherein the nAChR agonist is varenicline.

Embodiment I-5. The method of embodiment I-2, wherein the nAChR agonist is (R)-5-((E)-2-pyrrolidin-3-ylvinyl)pyrimidine, or a pharmaceutically acceptable salt thereof.

Embodiment I-6. The method of any one of embodiments I-1 to I-5, wherein the treatment that increases tear production is administered via local nasal administration.

Embodiment I-7. The method of embodiment I-6, wherein the local nasal administration is via an intranasal spray.

Embodiment I-8. The method of embodiment I-1, wherein the treatment that increases tear production is an effective electrical stimulation of the anterior ethmoid nerve.

Embodiment I-9. The method of any one of embodiments I-1 to I-8, wherein the method results an increased amount of the primary therapeutic agent delivered to the tear film of an eye of the subject in a predetermined time compared to administration of the primary therapeutic agent alone.

Embodiment I-10. The method of any one of embodiments I-1 to I-9, wherein the method results an increased amount of the primary therapeutic agent delivered to the cornea of an eye of the subject in a predetermined time compared to administration of the primary therapeutic agent alone.

Embodiment I-11. The method of embodiment I-9 or I-10, wherein the predetermined time is about 5 minutes.

Embodiment I-12. The method of embodiment I-9 or I-10, wherein the predetermined time is about 1 hour.

Embodiment I-13. The method of any one of embodiments I-1 to I-12, wherein administering an effective amount of the primary therapeutic agent comprises administering an effective amount of a composition comprising a viral vector comprising a polynucleotide encoding a gene product, or functional variant thereof, to at least one lacrimal gland of the subject, wherein the primary therapeutic agent comprises the gene product, or functional variant thereof.

Embodiment I-14. The method of embodiment I-13, wherein the gene product is a therapeutic protein.

Embodiment I-15. The method of embodiment I-14, wherein the therapeutic protein is nerve growth factor (NGF).

Embodiment I-16. The method of any one of embodiments I-13 to I-15, wherein the viral vector is an adeno-associated virus (AAV) vector.

Embodiment I-17. The method of any one of embodiments I-13 to I-15, wherein the viral vector is a vaccinia virus vector.

Embodiment I-18. The method of any one of embodiments I-13 to I-15, wherein the viral vector is an adenovirus vector.

Embodiment I-19. The method of any one of embodiments I-13 to I-15, wherein the viral vector is a lentiviral vector.

Embodiment I-20. The method of any one of embodiments I-13 to I-16, wherein the AAV vector is an AAV of serotype 5 (AAV5), serotype 2 (AAV2), serotype 8 (AAV8), or serotype 9 (AAV9).

Embodiment I-21. The method of any one of embodiments I-13 to I-16 or I-20, wherein the composition comprises about 1×10¹² genome copies per milliliter (GC/mL) of the AAV vector.

Embodiment I-22. The method of any one of embodiments I-13 to I-16 or I-20, wherein the composition comprises about 6.2×10¹² genome copies per milliliter (GC/mL) of the AAV vector.

Embodiment I-23. The method of any one of embodiments I-13 to I-22, wherein about 100 μL of the composition is administered.

Embodiment I-24. The method of any one of embodiments I-13 to I-23, wherein the method results in expression of the gene product in the tear film of an eye of the subject

Embodiment I-25. The method of any one of embodiments I-13 to I-24, wherein the method results in expression of the gene product in the cornea of an eye of the subject.

Embodiment I-26. The method of any one of embodiments I-13 to I-25, wherein the method results an increased amount of the gene product delivered to the tear film in a predetermined time compared to administration of the AAV vector alone.

Embodiment I-27. The method of embodiment I-26, wherein the predetermined time is about 5 minutes.

Embodiment I-28. The method of embodiment I-26, wherein the predetermined time is about 1 hour.

Embodiment I-29. A method of delivering a gene product to the ocular surface at least one eye of a subject in need thereof, comprising:
a. administering an effective amount of a composition comprising a viral vector comprising a polynucleotide encoding a gene product, or functional variant thereof, to at least one lacrimal gland of the subject; and
b. administering via local nasal administration an effective amount of a nicotinic acetylcholine receptor (nAChR) agonist, or a pharmaceutically acceptable salt thereof,

Embodiment I-30. The method of embodiment I-29, wherein the gene product is a therapeutic protein.

Embodiment I-31. The method of embodiment I-30, wherein the therapeutic protein is nerve growth factor (NGF).

Embodiment I-32. The method of any one of embodiments I-29 to I-31, wherein the viral vector is an adeno-associated virus (AAV) vector.

Embodiment I-33. The method of any one of embodiments I-29 to I-31, wherein the viral vector is a vaccinia virus vector.

Embodiment I-34. The method of any one of embodiments I-29 to I-31, wherein the viral vector is an adenovirus vector.

Embodiment I-35. The method of any one of embodiments I-29 to I-31, wherein the viral vector is a lentiviral vector.

Embodiment I-36. The method of embodiment I-32, wherein the AAV vector is an AAV of serotype 5 (AAV5), serotype 2 (AAV2), serotype 8 (AAV8), or serotype 9 (AAV9).

Embodiment I-37. The method of embodiment I-32 or I-36, wherein the composition comprises about 1×10¹² genome copies per milliliter (GC/mL) of the AAV vector.

Embodiment I-38. The method of embodiment I-32 or I-36, wherein the composition comprises about 6.2×10¹² genome copies per milliliter (GC/mL) of the AAV vector.

Embodiment I-39. The method of any one of embodiments I-29 to I-38, wherein about 100 μL of the composition comprising a viral vector is administered.

Embodiment I-40. The method of any one of embodiments I-29 to I-39, wherein the method results in expression of the gene product in the tear film of an eye of the subject

Embodiment I-41. The method of any one of embodiments I-29 to I-40, wherein the method results in expression of the gene product in the cornea of an eye of the subject.

Embodiment I-42. The method of any one of embodiments I-29 to I-41, wherein the method results an increased amount of the gene product delivered to the tear film in a predetermined time compared to administration of the AAV vector alone.

Embodiment I-43. The method of embodiment I-42, wherein the predetermined time is about 5 minutes.

Embodiment I-44. The method of embodiment I-42, wherein the predetermined time is about 1 hour.

Embodiment I-45. The method of any one of embodiments I-29 to I-44, wherein the method results an increased amount of the gene product delivered to the ocular surface in a predetermined time compared to administration of the AAV vector alone.

Embodiment I-46. The method of embodiment I-45, wherein the predetermined time is about 5 minutes.

Embodiment I-47. The method of embodiment I-45, wherein the predetermined time is about 1 hour.

Embodiment I-48. The method of any one of embodiments I-29 to I-47, wherein the nAChR agonist is a full agonist of nAChR subtypes alpha4beta2, alpha3beta4, alpha3alpha5beta4, and/or alpha4alpha6beta2.

Embodiment I-49. The method of any one of embodiments I-29 to I-47, wherein the nAChR agonist is varenicline.

Embodiment I-50. The method of any one of embodiments I-29 to I-48, wherein the nAChR agonist is (R)-5-((E)-2-pyrrolidin-3-ylvinyl)pyrimidine.

Embodiment I-51. The method of any one of embodiments I-29 to I-50, wherein the nAChR agonist is administered via local nasal administration.

Some embodiments of this disclosure relate to Embodiment II, as follows:

Embodiment II-1. A nicotinic acetylcholine receptor (nAChR) agonist, or a pharmaceutically acceptable salt thereof, for use in a method of treating an ocular surface disorder, cornea disorder, or anterior chamber disorder in a subject, the method comprising administering to the subject an effective amount of the nAChR agonist or pharmaceutically acceptable salt thereof,
wherein the method further comprises administering a primary therapeutic agent to an eye of the subject or to a tissue in fluidic communication with the eye.

Embodiment II-2. The nAChR agonist or pharmaceutically acceptable salt thereof for use of embodiment II-1, wherein the nAChR agonist or pharmaceutically acceptable salt thereof is administered via local nasal administration.

Embodiment II-3. The nAChR agonist or pharmaceutically acceptable salt thereof for use of embodiment II-2, wherein the local nasal administration is via an intranasal spray.

Embodiment II-4. A combined preparation of:
a. a primary therapeutic agent; and
b. a treatment that increases tear production;
   for simultaneous, separate or sequential use in a method of treating an ocular surface disorder, cornea disorder, or anterior chamber disorder in a subject, the method comprising administering an effective amount of the primary therapeutic agent to an eye of the subject or to a tissue in fluidic communication with the eye, and administering the treatment that increases tear production.

Embodiment II-5. A primary therapeutic agent for use in a method of treating an ocular surface disorder, cornea disorder, or anterior chamber disorder in a subject, the method comprising administering an effective amount of the primary therapeutic agent to an eye of the subject or to a tissue in fluidic communication with the eye,
wherein the method further comprises administering to the subject a treatment that increases tear production.

Embodiment II-6. The combined preparation for use of embodiment II-4 or the primary therapeutic agent for use of embodiment II-5, wherein the treatment that increases tear production comprises an effective amount of a nicotinic acetylcholine receptor (nAChR) agonist, or a pharmaceutically acceptable salt thereof.

Embodiment II-7. The nAChR agonist or salt thereof for use of any one of embodiments II-1 to II-3, or the combined preparation or primary therapeutic agent for use of embodiment II-6, wherein the nAChR agonist is a full agonist of nAChR subtypes alpha4beta2, alpha3beta4, alpha3alpha5beta4, and/or alpha4alpha6beta2.

Embodiment II-8. The nAChR agonist or salt thereof for use of any one of embodiments II-1 to II-3, or the combined preparation or primary therapeutic agent for use of embodiment II-6, wherein the nAChR agonist is varenicline.

Embodiment II-9. The nAChR agonist or salt thereof for use of any one of embodiments II-1 to II-3, or the combined preparation or primary therapeutic agent for use of embodiment II-6, wherein the nAChR agonist is (R)-5-((E)-2-pyrrolidin-3-ylvinyl)pyrimidine, or a pharmaceutically acceptable salt thereof.

Embodiment II-10. The combined preparation or primary therapeutic agent for use of any one of embodiments II-4 to II-9, wherein the treatment that increases tear production is administered via local nasal administration.

Embodiment II-11. The combined preparation or primary therapeutic agent for use of embodiment II-10, wherein the local nasal administration is via an intranasal spray.

Embodiment II-12. The primary therapeutic agent of embodiment II-5, wherein the treatment that increases tear production is an effective electrical stimulation of the anterior ethmoid nerve.

Embodiment II-13. The nAChR agonist or salt thereof, the combined preparation, or the primary therapeutic agent for use of any one of embodiments II-1 to II-12, wherein the method results an increased amount of the primary therapeutic agent delivered to the tear film of an eye of the subject in a predetermined time compared to administration of the primary therapeutic agent alone.

Embodiment II-14. The nAChR agonist or salt thereof, the combined preparation, or the primary therapeutic agent for use of any one of embodiments II-1 to II-13, wherein the method results an increased amount of the primary therapeutic agent delivered to the cornea of an eye of the subject in a predetermined time compared to administration of the primary therapeutic agent alone.

Embodiment II-15. The nAChR agonist or salt thereof, the combined preparation, or the primary therapeutic agent for use of embodiment II-13 or II-14, wherein the predetermined time is about 5 minutes.

Embodiment II-16. The nAChR agonist or salt thereof, the combined preparation, or the primary therapeutic agent for use of embodiment II-13 or II-14, wherein the predetermined time is about 1 hour.

Embodiment II-17. The nAChR agonist or salt thereof, the combined preparation, or the primary therapeutic agent, for use of any one of embodiments II-1 to II-16, wherein administering an effective amount of the primary therapeutic agent comprises administering an effective amount of a composition comprising a viral vector comprising a polynucleotide encoding a gene product, or functional variant thereof, to at least one lacrimal gland of the subject, wherein the primary therapeutic agent comprises the gene product, or functional variant thereof.

Embodiment II-18. The nAChR agonist or salt thereof, the combined preparation, or the primary therapeutic agent for use of embodiment II-17, wherein the gene product is a therapeutic protein.

Embodiment II-19. The nAChR agonist or salt thereof, the combined preparation, or the primary therapeutic agent for use of embodiment II-18, wherein the therapeutic protein is nerve growth factor (NGF).

Embodiment II-20. The nAChR agonist or salt thereof, the combined preparation, or the primary therapeutic agent for use of any one of embodiments II-17 to II-19, wherein the viral vector is an adeno-associated virus (AAV) vector.

Embodiment II-21. The nAChR agonist or salt thereof, the combined preparation, or the primary therapeutic agent for use of any one of embodiments II-17 to II-19, wherein the viral vector is a vaccinia virus vector.

Embodiment II-22. The nAChR agonist or salt thereof, the combined preparation, or the primary therapeutic agent for use of any one of embodiments II-17 to II-19, wherein the viral vector is an adenovirus vector.

Embodiment II-23. The nAChR agonist or salt thereof, the combined preparation, or the primary therapeutic agent for use of any one of embodiments II-17 to II-19, wherein the viral vector is a lentiviral vector.

Embodiment II-24. The nAChR agonist or salt thereof, the combined preparation, or the primary therapeutic agent for use of any one of embodiments II-17 to II-20, wherein the AAV vector is an AAV of serotype 5 (AAV5), serotype 2 (AAV2), serotype 8 (AAV8), or serotype 9 (AAV9).

Embodiment II-25. The nAChR agonist or salt thereof, the combined preparation, primary therapeutic agent, or nAChR agonist or salt thereof for use of any one of embodiments II-17 to II-20 or II-24, wherein the composition comprises about 1×10¹² genome copies per milliliter (GC/mL) of the AAV vector.

Embodiment II-26. The nAChR agonist or salt thereof, the combined preparation, or the primary therapeutic agent for use of any one of embodiments II-17 to II-20 or II-24, wherein the composition comprises about 6.2×10¹² genome copies per milliliter (GC/mL) of the AAV vector.

Embodiment II-27. The nAChR agonist or salt thereof, the combined preparation, or the primary therapeutic agent for use of any one of embodiments II-17 to II-26, wherein about 100 μL of the composition is administered.

Embodiment II-28. The nAChR agonist or salt thereof, the combined preparation, or the primary therapeutic agent for use of any one of embodiments II-17 to II-27, wherein the method results in expression of the gene product in the tear film of an eye of the subject

Embodiment II-29. The nAChR agonist or salt thereof, the combined preparation, or the primary therapeutic agent for use of any one of embodiments II-17 to II-28, wherein the method results in expression of the gene product in the cornea of an eye of the subject.

Embodiment II-30. The nAChR agonist or salt thereof, the combined preparation, primary therapeutic agent, or nAChR agonist or salt thereof for use of any one of embodiments II-14 to II-16, wherein the method results an increased amount of the gene product delivered to the tear film in a predetermined time compared to administration of the AAV vector alone.

Embodiment II-31. The nAChR agonist or salt thereof, the combined preparation, or the primary therapeutic agent for use of embodiment II-30, wherein the predetermined time is about 5 minutes.

Embodiment II-32. The nAChR agonist or salt thereof, the combined preparation, or the primary therapeutic agent for use of embodiment II-30, wherein the predetermined time is about 1 hour.

Embodiment II-33. A nicotinic acetylcholine receptor (nAChR) agonist, or a pharmaceutically acceptable salt thereof, for use in a method of treating an ocular surface disorder, cornea disorder, or anterior chamber disorder in a subject by delivering a gene product to the ocular surface at least one eye of the subject, the method comprising administering an effective amount of the nicotinic acetylcholine receptor (nAChR) agonist or pharmaceutically acceptable salt thereof via local nasal administration,
wherein the method further comprises administering to at least one lacrimal gland of the subject an effective amount of a composition comprising a viral vector comprising a polynucleotide encoding a gene product, or functional variant thereof.

Embodiment II-34. A combined preparation of:
a. a composition comprising a viral vector comprising a polynucleotide encoding a gene product, or functional variant thereof; and
b. a nicotinic acetylcholine receptor (nAChR) agonist, or a pharmaceutically acceptable salt thereof,
for simultaneous, separate or sequential use in a method of treating an ocular surface disorder, cornea disorder, or anterior chamber disorder in a subject by delivering a gene product to the ocular surface at least one eye of the subject, the method comprising administering an effective amount of the composition to at least one lacrimal gland of the subject, and administering an effective amount of the nicotinic acetylcholine receptor (nAChR) agonist or pharmaceutically acceptable salt thereof via local nasal administration.

Embodiment II-35. A composition comprising a viral vector comprising a polynucleotide encoding a gene product, or functional variant thereof, for use in a method of treating an ocular surface disorder, cornea disorder, or anterior chamber disorder in a subject by delivering a gene product to the ocular surface at least one eye of the subject, the method comprising administering an effective amount of the composition to at least one lacrimal gland of the subject,
wherein the method further comprises administering to the subject an effective amount of the nicotinic acetylcholine receptor (nAChR) agonist or pharmaceutically acceptable salt thereof via local nasal administration.

Embodiment II-36. The nAChR agonist or salt thereof for use of embodiment II-33, the combined preparation for use of embodiment II-34, the composition for use of embodiment II-35, wherein the gene product is a therapeutic protein.

Embodiment II-37. The nAChR agonist or salt thereof, the combined preparation, or the composition for use of embodiment II-36, wherein the therapeutic protein is nerve growth factor (NGF).

Embodiment II-38. The nAChR agonist or salt thereof, the combined preparation, or the composition for use of any one of embodiments II-33 to II-37, wherein the viral vector is an adeno-associated virus (AAV) vector.

Embodiment II-39. The nAChR agonist or salt thereof, the combined preparation, or the composition for use of any one of embodiments II-33 to II-37, wherein the viral vector is a vaccinia virus vector.

Embodiment II-40. The nAChR agonist or salt thereof, the combined preparation, or the composition for use of any one of embodiments II-33 to II-37, wherein the viral vector is an adenovirus vector.

Embodiment II-41. The nAChR agonist or salt thereof, the combined preparation, or the composition for use of any one of embodiments II-33 to II-37, wherein the viral vector is a lentiviral vector.

Embodiment II-42. The nAChR agonist or salt thereof, the combined preparation, or the composition for use of embodiment II-38, wherein the AAV vector is an AAV of serotype 5 (AAV5), serotype 2 (AAV2), serotype 8 (AAV8), or serotype 9 (AAV9).

Embodiment II-43. The nAChR agonist or salt thereof, the combined preparation, or the composition for use of embodiment II-38 or II-42, wherein the composition comprises about 1×10¹² genome copies per milliliter (GC/mL) of the AAV vector.

Embodiment II-44. The nAChR agonist or salt thereof, the combined preparation, or the composition for use of embodiment II-38 or II-42, wherein the composition comprises about 6.2×10¹² genome copies per milliliter (GC/mL) of the AAV vector.

Embodiment II-45. The nAChR agonist or salt thereof, the combined preparation, or the composition for use of any one of embodiments II-33 to II-44, wherein about 100 μL of the composition comprising a viral vector is administered.

Embodiment II-46. The nAChR agonist or salt thereof, the combined preparation, or the composition for use of any one of embodiments II-33 to II-45, wherein the method results in expression of the gene product in the tear film of an eye of the subject

Embodiment II-47. The nAChR agonist or salt thereof, the combined preparation, or the composition for use of any one of embodiments II-33 to II-46, wherein the method results in expression of the gene product in the cornea of an eye of the subject.

Embodiment II-48. The nAChR agonist or salt thereof, the combined preparation, or the composition for use of any one of embodiments II-33 to II-47, wherein the method results an increased amount of the gene product delivered to the tear film in a predetermined time compared to administration of the AAV vector alone.

Embodiment II-49. The nAChR agonist or salt thereof, the combined preparation, or the composition for use of embodiment II-48, wherein the predetermined time is about 5 minutes.

Embodiment II-50. The nAChR agonist or salt thereof, the combined preparation, or the composition for use of embodiment II-48, wherein the predetermined time is about 1 hour.

Embodiment II-51. The nAChR agonist or salt thereof, the combined preparation, or the composition for use of any one of embodiments II-33 to II-50, wherein the method results an increased amount of the gene product delivered to the ocular surface in a predetermined time compared to administration of the AAV vector alone.

Embodiment II-52. The nAChR agonist or salt thereof, the combined preparation, or the composition for use of embodiment II-51, wherein the predetermined time is about 5 minutes.

Embodiment II-53. The nAChR agonist or salt thereof, the combined preparation, or the composition for use of embodiment II-51, wherein the predetermined time is about 1 hour.

Embodiment II-54. The nAChR agonist or salt thereof, the combined preparation, or the composition for use of any one of embodiments II-33 to II-53, wherein the nAChR agonist is a full agonist of nAChR subtypes alpha4beta2, alpha3beta4, alpha3alpha5beta4, and/or alpha4alpha6beta2.

Embodiment II-55. The nAChR agonist or salt thereof, the combined preparation, or the composition for use of any one of embodiments II-33 to II-53, wherein the nAChR agonist is varenicline.

Embodiment II-56. The nAChR agonist or salt thereof, the combined preparation, or the composition for use of any one of embodiments II-33 to II-54, wherein the nAChR agonist is (R)-5-((E)-2-pyrrolidin-3-ylvinyl)pyrimidine.

Some embodiments of this disclosure relate to Embodiment III, as follows:

Embodiment III-1. A method of treating an ocular surface disorder, cornea disorder, or anterior chamber disorder in a subject, comprising:
a. administering an effective amount of a primary therapeutic agent to the eye or a tissue in fluidic communication with the eye; and
b. administering a treatment that increases tear production.

Embodiment III-2. The method of embodiment III-1, wherein the treatment that increases tear production comprises an effective amount of a nicotinic acetylcholine receptor (nAChR) agonist, or a pharmaceutically acceptable salt thereof.

Embodiment III-3. The method of embodiment III-2, wherein the nAChR agonist is a full agonist of nAChR subtypes alpha4beta2, alpha3beta4, alpha3alpha5beta4, and/or alpha4alpha6beta2.

Embodiment III-4. The method of embodiment III-2, wherein the nAChR agonist is varenicline, or a pharmaceutically acceptable salt thereof.

Embodiment III-5. The method of embodiment III-2, wherein the nAChR agonist is (R)-5-((E)-2-pyrrolidin-3-ylvinyl)pyrimidine, or a pharmaceutically acceptable salt thereof.

Embodiment III-6. The method of any one of embodiments III-1 to III-5, wherein the treatment that increases tear production is administered via local nasal administration.

Embodiment III-7. The method of embodiment III-6, wherein the local nasal administration is via an intranasal spray.

Embodiment III-8. The method of any one of embodiments III-1 to III-7, wherein the primary therapeutic agent is administered via intralacrimal injection into one or both lacrimal glands or topical administration.

Embodiment III-9. The method of any one of embodiments III-1 to III-8, wherein the treatment that increases tear production is administered subsequent to administration of the primary therapeutic agent.

Embodiment III-10. The method of embodiment III-9, wherein the treatment that increases tear production is administered beginning 7 days subsequent to administration of the primary therapeutic agent.

Embodiment III-11. The method of embodiment III-1, wherein the treatment that increases tear production is an effective electrical stimulation of the anterior ethmoid nerve.

Embodiment III-12. The method of any one of embodiments III-1 to III-11, wherein the tissue in fluidic communication with the eye is one or any combination of the lacrimal gland, the lacrimal duct, the cornea, cornea epithelium, limbal stem cells, conjunctiva, lacrimal puncta, and lacrimal canaliculi.

Embodiment III-13. The method of any one of embodiments III-1 to III-12, wherein a tissue in fluidic communication with the eye is the cornea.

Embodiment III-14. The method of any one of embodiments III-1 to III-12, wherein a tissue in fluidic communication with the eye is the lacrimal gland.

Embodiment III-15. The method of any one of embodiments III-1 to III-14, wherein the method results an increased amount of the primary therapeutic agent delivered to the tear film of an eye of the subject in a predetermined time compared to administration of the primary therapeutic agent alone.

Embodiment III-16. The method of any one of embodiments III-1 to III-13 or III-15, wherein the method results in an increased amount of the primary therapeutic agent delivered to the cornea of an eye of the subject in a predetermined time compared to administration of the primary therapeutic agent alone.

Embodiment III-17. The method of embodiment III-15 or III-16, wherein the predetermined time is about 5 minutes.

Embodiment III-18. The method of embodiment III-15 or III-16, wherein the predetermined time is about 1 hour.

Embodiment III-19. The method of any one of embodiments III-1 to III-18, wherein administering an effective amount of the primary therapeutic agent comprises administering an effective amount of a composition comprising a viral vector comprising a polynucleotide encoding a gene product, or functional variant thereof, to at least one lacrimal gland of the subject, wherein the primary therapeutic agent comprises the gene product, or functional variant thereof.

Embodiment III-20. The method of embodiment III-19, wherein the gene product is a therapeutic protein.

Embodiment III-21. The method of embodiment III-20, wherein the therapeutic protein is Nerve Growth Factor (NGF).

Embodiment III-22. The method of embodiment III-20, wherein the therapeutic protein is Glial Derived Neurotrophic Factor (GDNF).

Embodiment III-23. The method of any one of embodiments III-19 to III-22, wherein the viral vector is an adeno-associated virus (AAV) vector.

Embodiment III-24. The method of any one of embodiments III-19 to III-22, wherein the viral vector is a vaccinia virus vector.

Embodiment III-25. The method of any one of embodiments III-19 to III-22, wherein the viral vector is an adenovirus vector.

Embodiment III-26. The method of any one of embodiments III-19 to III-22, wherein the viral vector is a lentiviral vector.

Embodiment III-27. The method of embodiment III-23, wherein the AAV vector is an AAV of serotype 5 (AAV5), serotype 2 (AAV2), serotype 8 (AAV8), or serotype 9 (AAV9).

Embodiment III-28. The method of embodiment III-23 or III-27, wherein the composition comprises about 1×10¹² genome copies per milliliter (GC/mL) of the AAV vector.

Embodiment III-29. The method of embodiment III-23 or III-27, wherein the composition comprises about 6.2×10¹² genome copies per milliliter (GC/mL) of the AAV vector.

Embodiment III-30. The method of any one of embodiments III-19 to III-29, wherein about 50 μL to about 100 μL of the composition is administered.

Embodiment III-31. The method of any one of embodiments III-19 to III-30, wherein the method results in expression of the gene product in the tear film of an eye of the subject

Embodiment III-32. The method of any one of embodiments III-19 to III-31, wherein the method results in expression of the gene product in the cornea of an eye of the subject.

Embodiment III-33. The method of any one of embodiments III-19 to III-32, wherein the method results an increased amount of the gene product delivered to the tear film in a predetermined time compared to administration of the viral vector alone.

Embodiment III-34. The method of embodiment III-33, wherein the predetermined time is about 5 minutes.

Embodiment III-35. The method of embodiment III-33, wherein the predetermined time is about 1 hour.

Embodiment III-36. The method of any one of embodiments III-1 to III-35, wherein the ocular surface disorder, cornea disorder, or anterior chamber disorder in the subject is neurotrophic keratitis, chemical burn of the ocular surface, corneal wound, corneal ulcer, persistent epithelial defect, dry eye disease, herpes simplex viral infection of the trigeminal nerve and/or the eye, varicella zoster virus infection of the trigeminal nerve and/or the eye, or diabetic complications of the corneal nerves.

Embodiment III-37. A method of delivering a gene product to the ocular surface at least one eye of a subject in need thereof, comprising:
a. administering an effective amount of a composition comprising a viral vector comprising a polynucleotide encoding a gene product, or functional variant thereof, to at least one lacrimal gland of the subject; and
b. administering via local nasal administration an effective amount of a nicotinic acetylcholine receptor (nAChR) agonist, or a pharmaceutically acceptable salt thereof.

Embodiment III-38. The method of embodiment III-37, wherein the gene product is a therapeutic protein.

Embodiment III-39. The method of embodiment III-38, wherein the therapeutic protein is Nerve Growth Factor (NGF).

Embodiment III-40. The method of embodiment III-38, wherein the therapeutic protein is Glial Derived Neurotrophic Factor (GDNF)

Embodiment III-41. The method of any one of embodiments III-37 to III-40, wherein the viral vector is an adeno-associated virus (AAV) vector.

Embodiment III-42. The method of any one of embodiments III-37 to III-40, wherein the viral vector is a vaccinia virus vector.

Embodiment III-43. The method of any one of embodiments III-37 to III-40, wherein the viral vector is an adenovirus vector.

Embodiment III-44. The method of any one of embodiments III-37 to III-40, wherein the viral vector is a lentiviral vector.

Embodiment III-45. The method of embodiment III-41, wherein the AAV vector is an AAV of serotype 5 (AAV5), serotype 2 (AAV2), serotype 8 (AAV8), or serotype 9 (AAV9).

Embodiment III-46. The method of embodiment III-41 or III-45, wherein the composition comprises about 1×10¹² genome copies per milliliter (GC/mL) of the AAV vector.

Embodiment III-47. The method of embodiment III-41 or III-45, wherein the composition comprises about 6.2×10¹² genome copies per milliliter (GC/mL) of the AAV vector.

Embodiment III-48. The method of any one of embodiments III-37 to III-47, wherein about 50 μL to about 100 μL of the composition comprising a viral vector is administered.

Embodiment III-49. The method of any one of embodiments III-37 to III-48, wherein the treatment that increases tear production is administered subsequent to administration of the primary therapeutic agent.

Embodiment III-50. The method of embodiment III-49, wherein the treatment that increases tear production is administered beginning 7 days subsequent to administration of the primary therapeutic agent.

Embodiment III-51. The method of any one of embodiments III-37 to III-50, wherein the method results in expression of the gene product in the tear film of an eye of the subject.

Embodiment III-52. The method of any one of embodiments III-37 to III-50, wherein the method results in expression of the gene product in the cornea of an eye of the subject.

Embodiment III-53. The method of any one of embodiments III-37 to III-52, wherein the method results an increased amount of the gene product delivered to the tear film in a predetermined time compared to administration of the viral vector alone.

Embodiment III-54. The method of embodiment III-53, wherein the predetermined time is about 5 minutes.

Embodiment III-55. The method of embodiment III-53, wherein the predetermined time is about 1 hour.

Embodiment III-56. The method of any one of embodiments III-37 to III-52, wherein the method results an increased amount of the gene product delivered to the ocular surface in a predetermined time compared to administration of the viral vector alone.

Embodiment III-57. The method of embodiment III-56, wherein the predetermined time is about 5 minutes.

Embodiment III-58. The method of embodiment III-56, wherein the predetermined time is about 1 hour.

Embodiment III-59. The method of any one of embodiments III-37 to III-58, wherein the nAChR agonist is a full agonist of nAChR subtypes alpha4beta2, alpha3beta4, alpha3alpha5beta4, and/or alpha4alpha6beta2.

Embodiment III-60. The method of any one of embodiments III-37 to III-58, wherein the nAChR agonist is varenicline, or a pharmaceutically acceptable salt thereof.

Embodiment III-61. The method of any one of embodiments III-37 to III-59, wherein the nAChR agonist is (R)-5-((E)-2-pyrrolidin-3-ylvinyl)pyrimidine, or a pharmaceutically acceptable salt thereof.

Embodiment III-62. The method of any one of embodiments III-37 to III-61, wherein the nAChR agonist is administered via local nasal administration.

Embodiment III-63. The method of any one of embodiments III-37 to III-62, wherein the composition comprising a viral vector is administered via intralacrimal injection.

Embodiment III-64. The method of any one of embodiments III-37 to III-63, wherein the ocular surface disorder, cornea disorder, or anterior chamber disorder in the subject is neurotrophic keratitis, chemical burn of the ocular surface, corneal wound, corneal ulcer, persistent epithelial defect, dry eye disease, herpes simplex viral infection of the trigeminal nerve and/or the eye, varicella zoster virus infection of the trigeminal nerve and/or the eye, or diabetic complications of the corneal nerves.

Embodiment III-65. A primary therapeutic agent and a treatment for increasing tear production for use in a method of treating an ocular surface disorder, cornea disorder, or anterior chamber disorder in a subject, the method comprising:
a. administering an effective amount of the primary therapeutic agent to the eye or a tissue in fluidic communication with the eye; and
b. administering the treatment that increases tear production.

Embodiment III-66. The primary therapeutic agent and treatment for increasing tear production for use of embodiment III-65, wherein the treatment that increases tear production comprises an effective amount of a nicotinic acetylcholine receptor (nAChR) agonist, or a pharmaceutically acceptable salt thereof.

Embodiment III-67. The primary therapeutic agent and treatment for increasing tear production for use of embodiment III-66, wherein the nAChR agonist is varenicline, or a pharmaceutically acceptable salt thereof.

Embodiment III-68. The primary therapeutic agent and treatment for increasing tear production for use of any one of embodiments III-65 to III-67, wherein the treatment that increases tear production is administered via local nasal administration.

Embodiment III-69. The primary therapeutic agent and treatment for increasing tear production of any one of embodiments III-65 to III-67, wherein administering an effective amount of the primary therapeutic agent comprises administering an effective amount of a composition comprising a viral vector comprising a polynucleotide encoding a gene product, or functional variant thereof, to at least one lacrimal gland of the subject, wherein the primary therapeutic agent comprises the gene product, or functional variant thereof.

Embodiment III-70. A primary therapeutic agent and treatment for increasing tear production for use in the manufacture of a medicament for treatment of an ocular disease, disorder, or condition.

Embodiment III-71. A kit comprising:
(a) a first composition comprising a viral vector comprising a polynucleotide encoding a gene product, or functional variant thereof; and
(b) a second composition comprising a nicotinic acetylcholine receptor (nAChR) agonist,
   and instructions for treating or delaying progression of a disease, disorder, or condition described herein in a subject in need thereof.

### EXAMPLES

The following specific examples are to be construed as merely illustrative, and not limitative of the remainder of the disclosure in any way whatsoever.

### Example 1: Expression of GFP Transgene in Lacrimal Gland Delivered by rAAV Via Intralacrimal Injection

This example shows a 9-day pilot study of single dose rAAV virions administered as an Intralacrimal Gland Injection to Dutch-Belted Rabbits. It evaluates the effectiveness and tolerability of a panel of rAAV virion embodiments administered one time via injection to the lacrimal gland. Each rAAV virion composition in the panel is tested at two concentrations (1×10¹² GC/mL and 6.2×10¹² GC/mL). The panel of rAAV virions include embodiments with capsid proteins having AAV2, AAV5, AAV8, and AAV9 serotypes. The expression cassette delivered by the rAAV virion encodes an enhanced green fluorescent protein (eGFP) transgene that is operatively linked to a CAG promoter. This approach evaluated the feasibility of capsid protein serotypes AAV2, AAV5, AAV8, and AAV9 to deliver a transgene to cells in the lacrimal gland resulting in measurable CAG promoter-driven expression of the transgene within the cells.

Animal studies were carried out in the Charles River Laboratories (CRL) facilities by CRL staff scientific personnel.

### Animal Test System, Husbandry, and In-Life Monitoring

The animals used in this study were male Dutch-Belted rabbits between the ages of 4 to 5 months and weighed between 1.3 to 2.3 kg. Animals were acclimated for 10 days prior to the start of treatment. Each animal was housed individually and cared for using standard caregiving protocols including regular environmental conditions, feeding schedules, and veterinary care.

rAAV Virion Compositions and Formulations

In this study, the panel of compositions comprising rAAV virions containing an expression cassette encoding an eGFP transgene operatively linked to a CAG promoter are provided for intralacrimal injection under the conditions in Table 4. Each composition contains an rAAV virion with a different AAV capsid protein serotype. The compositions are labeled as OC-100a-d, each corresponding to a different AAV capsid protein serotype. Dose formulations for intralacrimal injection were prepared using clean procedures at the target concentrations described below in Table 4 by diluting with phosphate buffered saline solution.

TABLE 4
Summary of rAAV Compositions

| | rAAV | rAAV | rAAV | rAAV |
|---|---|---|---|---|
| | Composition 1 | Composition 2 | Composition 3 | Composition 4 |
| | (OC-100a) | (OC-100b) | (OC-100c) | (OC-100d) |
| AAV Capsid | AAV2 | AAV5 | AAV8 | AAV9 |
| Serotype | | | | |
| Physical | Clear Liquid | Clear Liquid | Clear Liquid | Clear Liquid |
| Description | | | | |
| Concentration | 1.5 × 10¹³ | 1.3 × 10¹³ | 1.1 × 10¹³ | 4 × 10¹³ |
| | GC/mL | GC/mL | GC/mL | GC/mL |
| Storage | −80° C. | −80° C. | −80° C. | −80° C. |
| Conditions | | | | |

Intralacrimal Injection of rAAV Compositions

Animals were dosed via intralacrimal injection on day 1 of the study. A summary of the formulation concentration for each composition tested, dose volume, dose frequency, and number of animals and lacrimal glands is found in Table 5. Prior to the injection, animals were anesthetized by intramuscular injection of dexmedetomidine (0.25 mg/kg) followed by an isoflurane/oxygen mix through a mask to maintain anesthesia, if necessary. A topical antibiotic was applied to each eye after dose administration.

TABLE 5
Summary of Experimental Design

| | | Formulation | | | | Number of |
|---|---|---|---|---|---|---|
| | | Concentration | Dose | | | Lacrimal |
| Group | rAAV | (genome copies | Volume | Dose | Number of | Glands |
| No. | Composition | (GC)/mL) | (μL/gland/dose) | Frequency | Animals | Injected |
| 1 | OC-100a | 1 × 10¹² | 100 | once | 1 | 2 |
| 2 | OC-100a | 6.2 × 10¹² | 100 | once | 2 | 4 |
| 3 | OC-100b | 1 × 10¹² | 100 | once | 1 | 2 |
| 4 | OC-100b | 6.2 × 10¹² | 100 | once | 2 | 4 |
| 5 | OC-100c | 1 × 10¹² | 100 | once | 1 | 2 |
| 6 | OC-100c | 6.2 × 10¹² | 100 | once | 2 | 4 |
| 7 | OC-100d | 1 × 10¹² | 100 | once | 1 | 2 |
| 8 | OC-100d | 6.2 × 10¹² | 100 | once | 2 | 4 |

### Bioanalysis

Blood was collected on Day 1 before dosing, and again on Day 8 and 9 from an auricular vessel from all animals. Blood samples were placed on ice until plasma was separated by centrifugation. Plasma samples were separated into 250 μL aliquots and frozen at −80° C. for subsequent analysis.

A Schirmer tear test was performed to collect eye moisture from the animals on Day 8 and 9. Test strips were placed inside the lower eyelid for approximately 1 minute. The paper was removed and placed into separate tubes and frozen at −80° C. for subsequent analysis.

Plasma samples and Schirmer's test strips were analyzed for concentration of eGFP and eGFP mRNA concentration using a validated procedure at Syneos analytical laboratories. Analysis in the plasma samples detected no eGFP protein or eGFP mRNA, indicating that intralacrimal delivery of the rAAV did not induce cell lysis. This result suggests the intralacrimal route of administration may be safely used for delivering rAAV.

### Immunohistochemistry of Lacrimal Gland Tissue

Animals were euthanized on day 9 following collection of blood and eye moisture by intravenous injection of sodium pentobarbital. Five sagittal sections of the left eye and sections of the left and right lacrimal glands were prepared for immunohistochemistry (IHC) according to Charles River lab standard operating procedure. Lacrimal gland IHC samples were stained for eGFP and subject to microscopic evaluation.

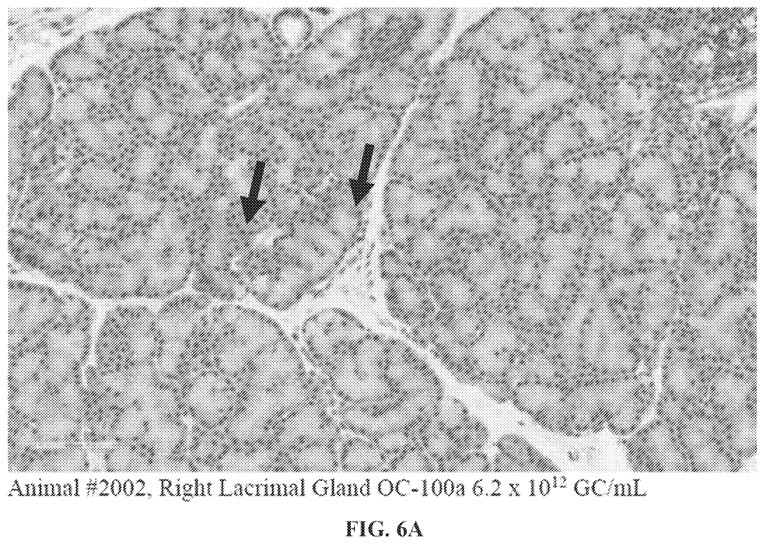

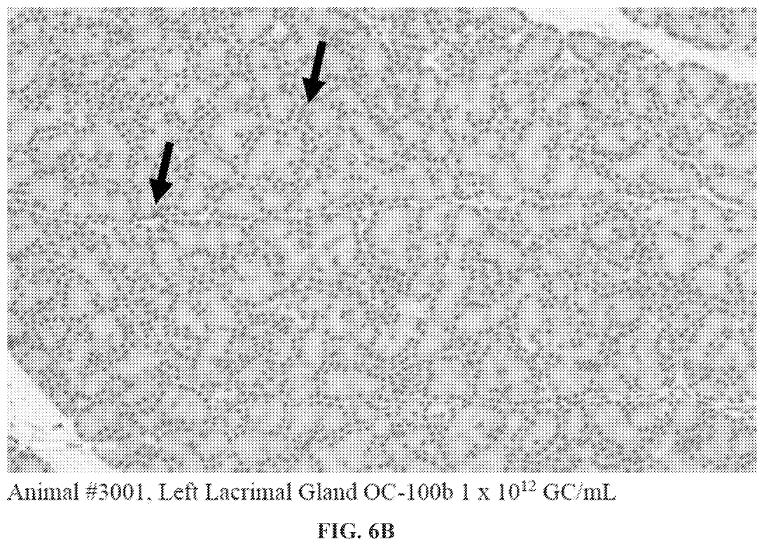

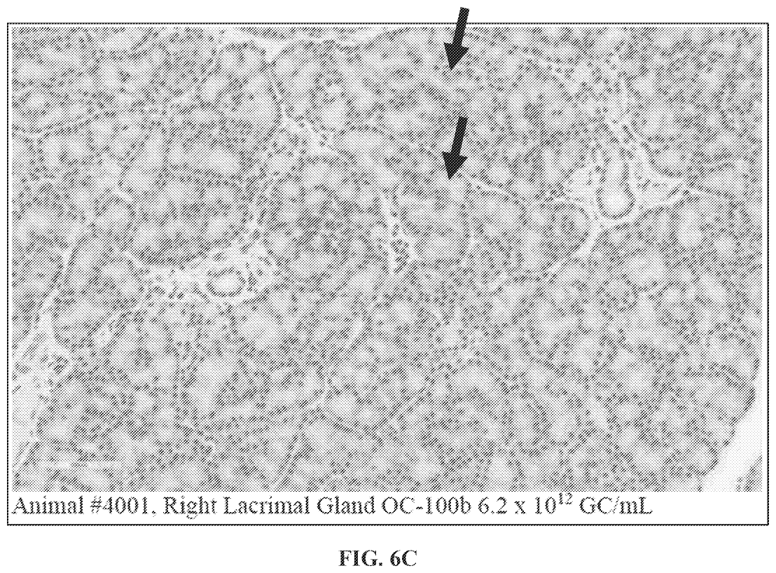

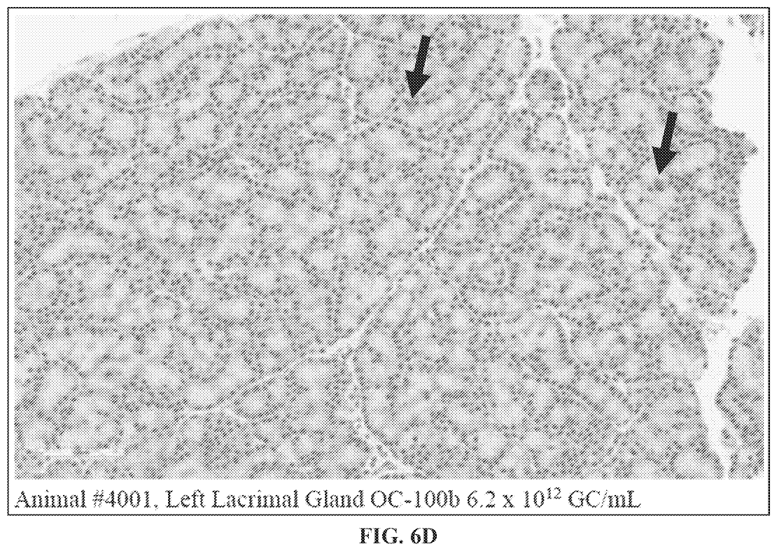

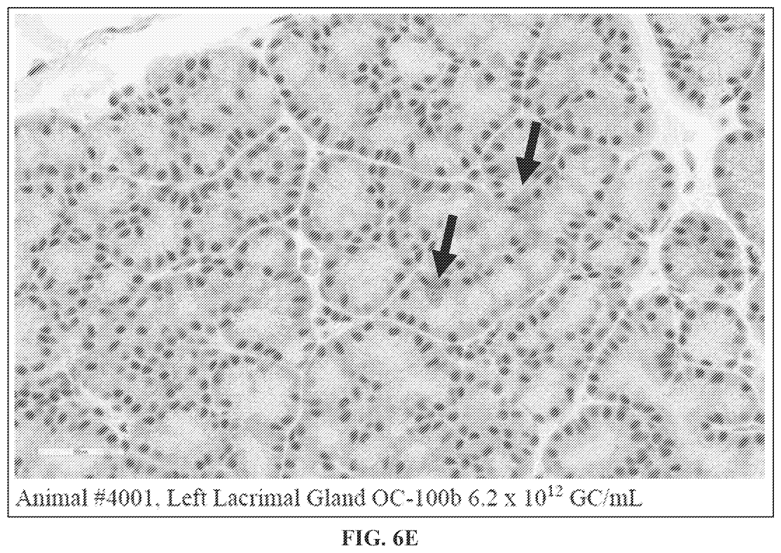

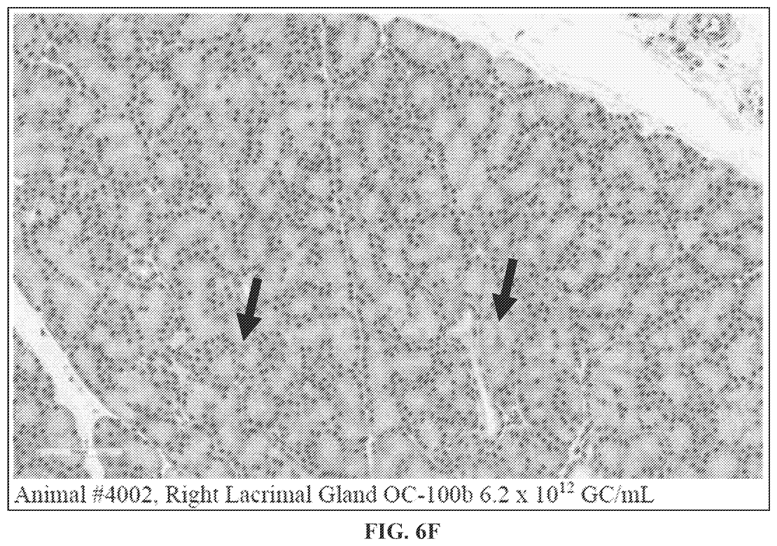

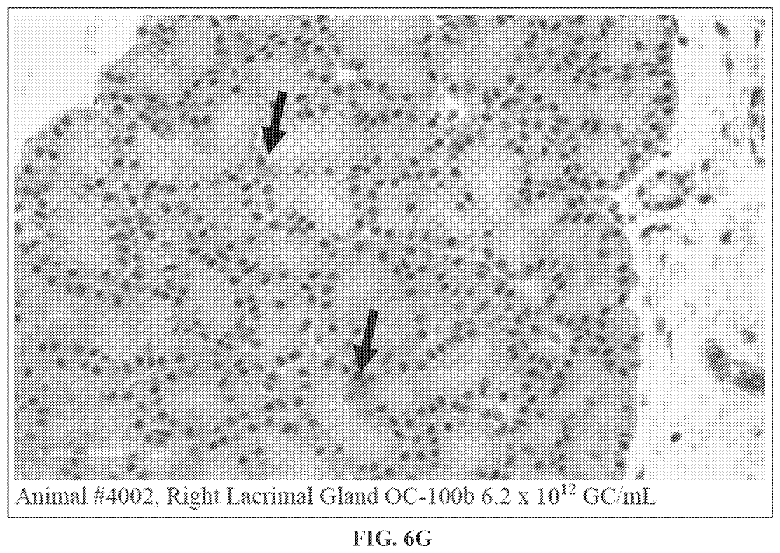

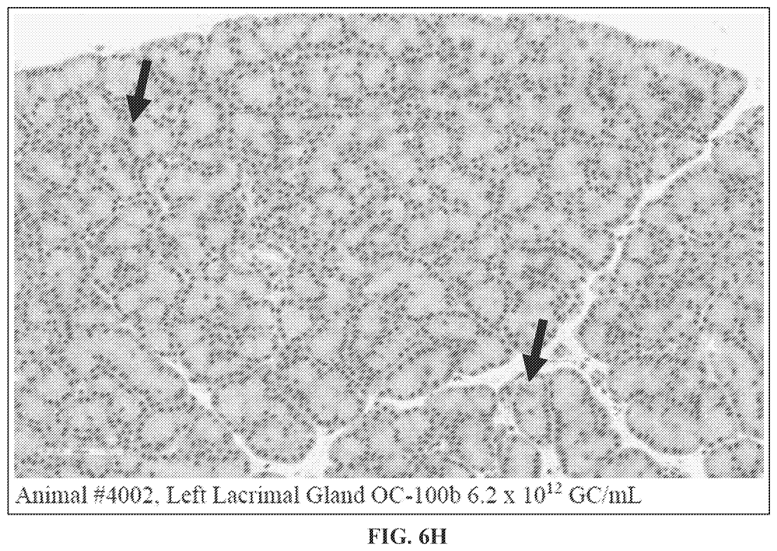

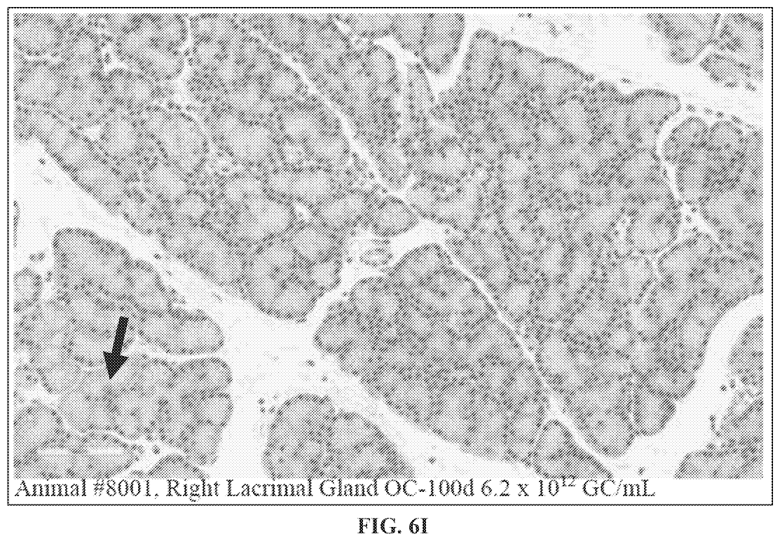

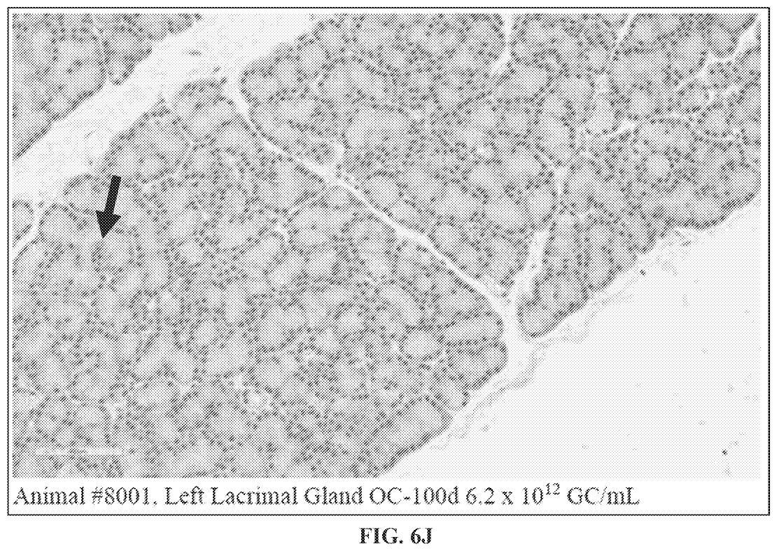

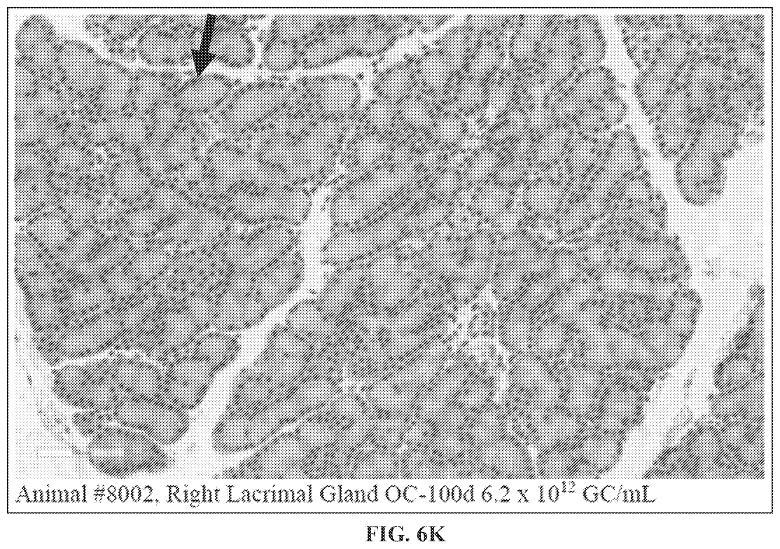

Microscopic evaluation was performed to determine the efficiency of eGFP expression in lacrimal gland tissues dosed in vivo with the rAAV compositions. Isolated positive acinar cells in the IHC samples had pink to red cytoplasmic staining indicative of GFP expression (FIGS. 6A-6K; exemplary staining indicated by black arrows). Positive eGFP expression was observed in for the rAAV composition containing an AAV2 capsid protein (OC-100a) at 6.2×10¹² GC/mL (FIG. 6A), the rAAV composition containing an AAV5 capsid protein at both 1×10¹² GC/mL (FIG. 6B) and 6.2×10¹² GC/mL (FIGS. 6C-6H), and the rAAV composition containing an AAV9 capsid protein at 6.2×10¹² GC/mL (FIGS. 6I-6K).

### Conclusion

These results from this example show that rAAV virions can be used to deliver an expression cassette to the lacrimal gland by direct injection. The results also show that rAAV virions containing capsid proteins with at least the AAV2, AAV5, or AAV9 serotypes can be used to deliver an expression cassette to cells within the lacrimal gland. Furthermore, the results demonstrate delivery of an expression cassette containing a transgene operatively linked to a CAG promoter sequence results in expression of the transgene in the cells of the lacrimal gland.

### Example 2. Expression of Neurotrophic Factors in Lacrimal Gland Delivered by rAAV Via Intralacrimal Injection

This example describes a seven-day, single-dose study of varenicline administered as a single intranasal dose to Dutch-Belted rabbits after an AAV vector is administered as an intra-lacrimal gland injection. It evaluates the tolerability and effectiveness of various AAV vectors at two dose levels (1×10¹²GC/mL or 6.2×10¹²GC/mL as a 100 μL injection) administered as an intra-Lacrimal gland injection followed by a single administration of varenicline nasal spray (1.2 mg/ml as a 50 μL spray) into each nostril of the subject. Specifically, this study design has two main objectives:
(1) To assess transduction of the lacrimal gland using adeno-associated virus vectors of serotype 2, 5, or 9 (AAV2, AAV5, AAV9) containing an expression cassette encoding either human nerve growth factor (hNGF) or human glial-derived neurotrophic factor (hGDNF) administered by intralacrimal injection. hNGF and hGDNF proteins are neutrophic factors that play a role in the maintenance and health of the corneal surface.
(2) To assess increasing the relative amount of protein present on the ocular surface by stimulating tear production with varenicline nasal spray.
   Varenicline is the following compound:

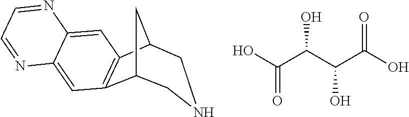 [SMILES: c1cnc2cc3c(cc2n1)C1CNCC3C1 ; O=C(O)[C@H](O)[C@@H](O)C(=O)O]

In this study an experimental viral vector or a control vector is administered to the lacrimal gland of Dutch-Belted Rabbits. The AAVx.hNGF and AAVx.hGDNF (x being 2, 5, or 9) vectors include a transgene encoding hNGF and hGDNF proteins, respectively. hNGF and hGDNF are illustrative therapeutic proteins to demonstrate feasibility for intralacrimal expression of AAV delivered transgene, whose secretion into tear film can be increased using a tear inducing agent, such as varenicline. AAVx.nNGF and AAVx.hGDNF is administered at a dose of 6.2×10¹¹ GC/mL. A vehicle control is used for comparison.

Animals are evaluated under anesthesia on Day 7 via slit lamp biomicroscopy using a blue light and appropriate filters. This evaluation is documented with slit lamp photography.

On Day 8, animals are administered a single 50 μL spray of 1.2 mg/ml varenicline nasal spray into each nostril. Immediately or within minutes after administration, animals are valuated under anesthesia via slit lamp biomicroscopy using a blue light and appropriate filters. This evaluation is documented with slit lamp photography.

### Methods

Dose administration (route): Intralacrimal injection, 100 μL into each lacrimal gland. Dose Frequency: Single injection into each lacrimal gland. Procedure: Under anesthesia, approximately 100 μL of Reference Item (PBS+0.001% F68), AAVx.hNGF (6.2×10¹¹GC/mL), or AAVx.hNGF (6.2×10¹¹GC/mL) is administered to each lacrimal gland under direct visualization. Lacrimal gland injection procedure is as follows: (1) a vertical incision is made underneath the right (OD) superior lid; (2) blunt dissection extends to the superior orbital rim until the lacrimal gland is visualized; (3) the lacrimal gland is injected with a 0.1 mL of Reference Item, AAVx.hNGF (6.2×10¹²GC/mL), or AAVx.hNGF (6.2×10¹²GC/mL) with a 1-mL sterile syringe with a 27 G ½-inch sterile needle; (4) the incision is closed with sutures/cyanoacrylate glue; and (5) the procedure is repeated for the left (OS) lacrimal gland. Gene therapy administration is summarized in Table 6.

TABLE 6
Administration of rAAVs

| | | Formulation | | | |
|---|---|---|---|---|---|
| | | Concentration | | | No. Animals |
| Group | | (genome | Dose Volume | Dose | (M/F) |
| No. | Dose | copies/volume) | (μL/animal/dose) | Frequency | Main Study |
| 1 | Reference Item 1 | NA | 100 μL injection | once | 2/2 |
| | | | into each gland | | |
| 2 | AAV2.hNGF | 6.2 × 10¹² GC/mL | 100 μL injection | once | 2/2 |
| | | | into each gland | | |
| 3 | AAV5.hNGF | 6.2 × 10¹² GC/mL | 100 μL injection | once | 2/2 |
| | | | into each gland | | |
| 4 | AAV9.hNGF | 6.2 × 10¹² GC/mL | 100 μL injection | once | 2/2 |
| | | | into each gland | | |
| 5 | AAV2.hGDNF | 6.2 × 10¹² GC/mL | 100 μL injection | once | 2/2 |
| | | | into each gland | | |
| 6 | AAV5.hGDNF | 6.2 × 10¹² GC/mL | 100 μL injection | once | 2/2 |
| | | | into each gland | | |
| 7 | AAV9.hGDNF | 6.2 × 10¹² GC/mL | 100 μL injection | once | 2/2 |
| | | | into each gland | | |

### Nasal Spray Tear Stimulation

Dose administration (route): Intranasal spray, both nostrils. Dose Frequency: One single administration; 50 μL spray once into each nostril; Day 8. Procedure: Animals is administered a 50 μL spray into each nostril utilizing an Aptar, Preservative Free nasal pump filled with varenicline solution 1.2 mg/ml. Termination: Day 8. Nasal spray tear stimulation is summarized in Table 7.

TABLE 7
Administration of varenicline

| | | | | | | No. |
|---|---|---|---|---|---|---|
| | | | Formulation | | | Animals |
| | | Dose | Concentration | Dose | | (M/F) |
| Group | | (mg/ | (weight/ | Volume^{a} | Dose | Main |
| No. | Test Item | animal) | volume) | (μL/animal/day) | Frequency | Study |
| 1 | Reference | 0.12 mg | NA | 100 μL | once | 2/2 |
| | Item 2 | | | | | |
| | (PBS + 0.001% | | | | | |
| | Pluronic | | | | | |
| | F-68) | | | | | |
| 2 | Varenicline | 0.12 mg | 1.2 mg/ml | 100 μL | once | 2/2 |
| 3 | Varenicline | 0.12 mg | 1.2 mg/ml | 100 μL | once | 2/2 |
| 4 | Varenicline | 0.12 mg | 1.2 mg/ml | 100 μL | once | 2/2 |
| 5 | Varenicline | 0.12 mg | 1.2 mg/ml | 100 μL | once | 2/2 |
| 6 | Varenicline | 0.12 mg | 1.2 mg/ml | 100 μL | once | 2/2 |
| 7 | Varenicline | 0.12 mg | 1.2 mg/ml | 100 μL | once | 2/2 |

^{a}The dose volume is administered to each animal, with the total volume to be administered 100 μL split equally between each nostril (50 μL per nostril per dose).

Transgene mRNA Quantification in the Lacrimal Tissue

The required section of lacrimal gland (0.005 g to ˜0.025 g) was immediately be placed in RNAlater® (fully immersed) and stored at room temperature and within 8 hours was transferred to a refrigerator set to maintain a temperature of 4° C. for overnight storage. On the following day, the RNAlater® was removed and the samples were stored frozen in a freezer set to maintain a temperature −80° C. The samples were analyzed for hNGF expression using RT-qPCR.

The samples of (0.005 g to 0.01 g) from the harderian gland, optic nerve, trigeminal nerve, nasal cavity via epithelial cell scraping, nictitating membrane, and extraocular muscles will be collected from the right side and placed in RNAlater (fully immersed) and stored at room temperature and within 8 hours will be transferred to a refrigerator set to maintain a temperature of 4° C. for overnight storage. On the following day, the RNAlater® will be removed and the samples will be stored frozen in a freezer set to maintain a temperature −80° ° C. for potential vg DNA and mRNA analysis.

### Histology for Immunohistochemistry (IHC)

The appropriate section of left and right lacrimal glands were harvested for morphologic and immunohistochemical evaluation. Samples were embedded in 4% paraformaldehyde and fixed in 70% ethanol. After paraffin blocking, 5-μm thick sections were obtained and mounted on a slide; for each sample one section was stained with H&E and immunohistochemistry staining were also performed. Staining of hNGF was accomplished using an anti-hNGF antibody. Slides were observed and images captured by microscope are assessed for hNGF protein expression.

### Schirmer Test

A Schirmer tear test was performed to collect eye moisture from the animals on Days 7, 14, 21, 28, 41, and 42. Test strips were placed inside the lower eyelid for approximately 1 minute. The paper was removed and placed into separate tubes and frozen at −80° C. for subsequent analysis. Schirmer test strips were collected following intranasal administration of varenicline on Days 21, 28, and 41. Test strips were placed inside the lower eyelid for approximately 1 minute. The paper was removed and placed into separate tubes and frozen at −80° ° C. for subsequent analysis. Protein content absorbed onto Schirmer's test strips was analyzed for concentration of hNGF by ELISA assay.

### Results

Quantification of transgene expression in the lacrimal gland is shown in Table 8.

TABLE 8
Expression of neurotrophic factors in lacrimal gland

| | | | No. | | | mRNA | | | |
|---|---|---|---|---|---|---|---|---|---|
| | | | Animals | | No. | (ss copies/μg total RNA) | | | |
| | | Formulation | Main | | Lacrimal | | | | |
| Group | | Dose Level | Study | | Glands | Mean | | St. Dev. | |
| No. | rAAV Vector | GC/gland/dose | M | F | Injected | RT | LT | RT | LT |
| 1 | Vehicle | NA | 2 | 2 | 8 | 0.0 | 0.0 | 0.0 | 0.0 |
| | Control | | | | | | | | |
| 2 | OC-101a | 6.2 × 10¹¹ | 2 | 2 | 8 | 10673.8 | 0.0 | 15505.4 | 0.0 |
| | (AAV2.hNGF) | | | | | | | | |
| 3 | OC-101b | 6.2 × 10¹¹ | 2 | 2 | 8 | 121597.6 | 156887.1 | 223042.3 | 286366.3 |
| | (AAV5.hNGF) | | | | | | | | |
| 4 | OC-101c | 6.2 × 10¹¹ | 2 | 2 | 8 | 18994.5 | 87761.3 | 31843.4 | 160533.3 |
| | (AAV9.hNGF) | | | | | | | | |
| 5 | OC-102a | 6.2 × 10¹¹ | 2 | 2 | 8 | 3674.2 | 1322.3 | 4906.5 | 1750.4 |
| | (AAV2.hGDNF) | | | | | | | | |
| 6 | OC-102b | 6.2 × 10¹¹ | 2 | 2 | 8 | 8817.6 | 124730.8 | 14948.1 | 249461.5 |
| | (AAV5.hGDNF) | | | | | | | | |
| 7 | OC-102c | 6.2 × 10¹¹ | 2 | 2 | 8 | 6271.2 | 3520.7 | 4671.0 | 3815.8 |
| | (AAV9.hGDNF) | | | | | | | | |

Tissues from all vehicle control animals tested negative for bGH gene expression, demonstrating that cross contamination was well controlled from animal dosing, sample collection, RNA extraction and RT-qPCR analysis. rAAV vector-derived hGDNF mRNAs were detected in all injected lacrimal gland tissues. For Groups 3 to 6, some injected lacrimal gland tissues had hNGF or hGDNF mRNAs from 1151 to 585,401 ss copies per μg of total RNA, while some others had no detectable vector-derived mRNA.

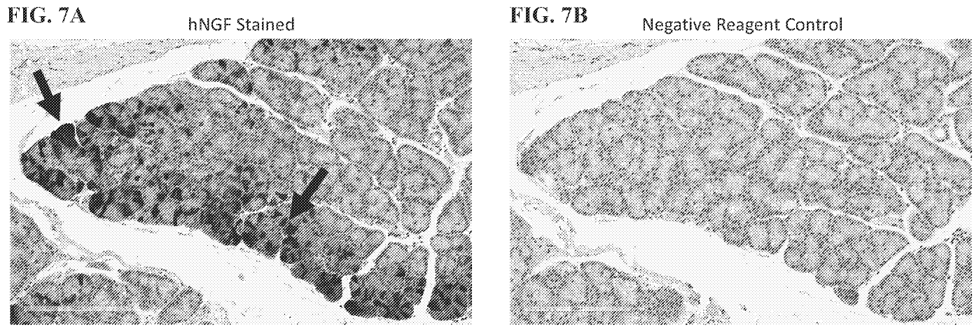

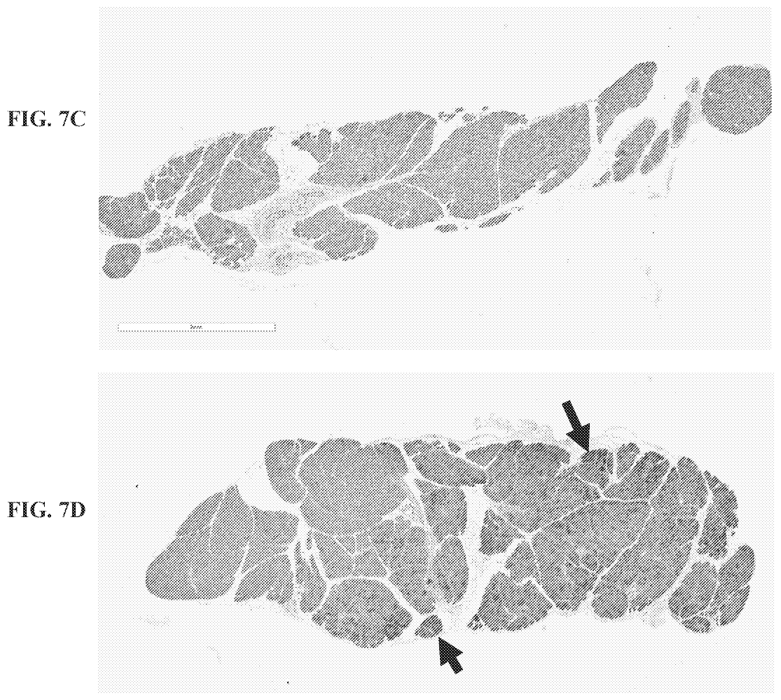

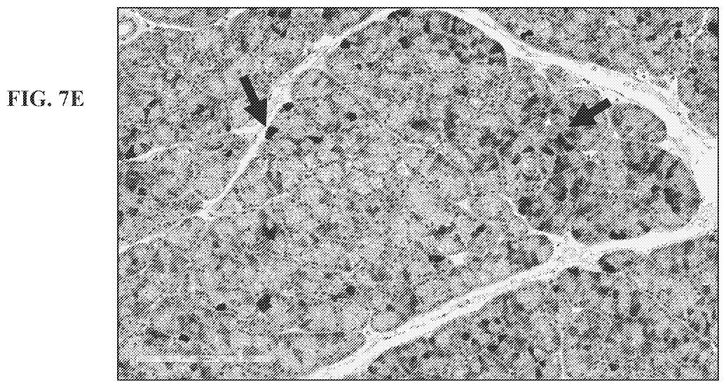

Lacrimal tissue was further assessed for hNGF expression using immunohistochemistry (FIGS. 7A-7E). Following intralacrimal injection, animals were euthanized and sections of the injected lacrimal glands were prepared for immunohistochemistry (IHC). Lacrimal gland IHC samples were stained for hNGF and subject to microscopic evaluation. Microscopic evaluation was performed to determine the efficiency of hNGF expression in lacrimal gland tissues dosed in vivo with the rAAV compositions. FIG. 7A and FIGS. 7D-7E shows illustrative examples of IHC staining in the lacrimal gland of rabbits injected with rAAV with an AAV9 serotype (exemplary staining shown by black arrows). The hNGF stained tissue shows strong expression of the hNGF transgene. The result is confirmed by comparing with the negative control sample (FIG. 7B and FIG. 7C), which shows little staining and thus rules out staining due to background. These results indicate intralacrimal injection of rAAV can be used to deliver and express a neurotrophic factor transgene, e.g., hNGF, in the lacrimal tissue.

A Schirmer tear test was performed to collect eye moisture from the animals on Days 7, 14, 21, 28, 41, and 42. Test strips were placed inside the lower eyelid for approximately 1 minute. The paper was removed and placed into separate tubes and frozen at −80° C. for subsequent analysis. Schirmer test strips were collected following intranasal administration of varenicline on Days 21, 28, and 41. Test strips were placed inside the lower eyelid for approximately 1 minute. The paper was removed and placed into separate tubes and frozen at −80° C. for subsequent analysis. Protein content absorbed onto Schirmer's test strips was analyzed for concentration of hNGF by ELISA assay.

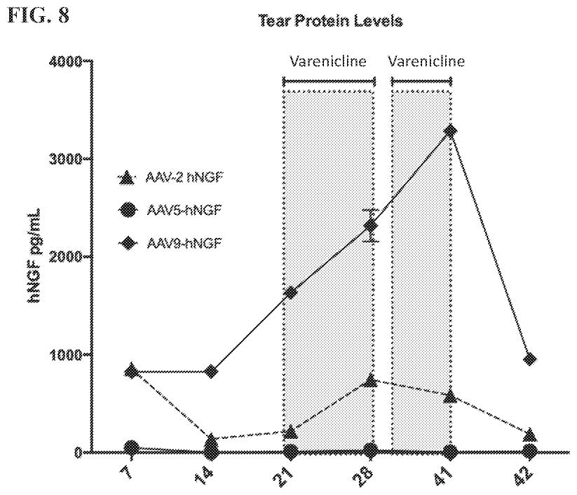

Schirmer's test strips were analyzed for concentration of hNGF in the collected tear film (FIG. 8). Following intralacrimal administration of rAAV9.hNGF, the hNGF concentration was constant at days 7 and 14 in the absence of intranasal administration of varenicline. On days 21, 28, and 41 the concentration of hNGF in the Schirmer strip increased up to about 3-fold over the levels measured at the 7 and 14 day collections, indicating that intranasal varenicline increased hNGF secretion into tear film. Notably, on day 42, in the absence of varenicline, a large drop in hNGF concentration was observed. hNGF concentration in the Schirmer strips was highest for animals administered rAAV9.hNGF than for rAAV5.hNGF or rAAV2.hNGF.

In summary, the results show that neurotrophic factors, such as hNGF and hGDNF, are expressed as transgenes when delivered via intralacrimal injection with rAAV, including AAV2, AAV5, and AAV9 serotypes, with AAV9 serotypes showing the best results. Further, intranasal delivery of a tear inducing agent, such as varenicline, increased the concentration of the expressed transgene in the tear film.

### Example 3. Detection of Human Nerve Growth Factor Beta Protein in Porcine Tears Following Transduction

Tears were collected from twelve pigs (Sus scrofa domesticus) seven (7), fourteen (14), twenty-one (21), twenty-eight (28), and thirty-five (35) days following transduction of lacrimal gland (via intralacrimal injection) using a single dose (2e11 vg/mL) of an adeno-associated viral vector (AAV) encoding human nerve growth factor (AAV-hNGFβ) utilizing adeno-associated virus serotypes 2 (n=4), 5 (n=4), and 9 (n=4) vectors. Collection of tear samples was performed on Day 7, 14, 21, 28, and 35 utilizing Schirmer Tear Test strips placed under the lower eyelid of both the right (OD) and left (OS) eyes individually for approximately 120 seconds in anesthetized animals. On Days 22-27, 0.03 mg varenicline solution was administered to each nostril twice a day (BID). For the Day 28 tear collection, 0.03 mg of varenicline solution was administered to each nostril via a nasal spray pump 2 minutes prior to tear collection. On all collection days, the Schirmer Tear Test strip was cut just above the fluid migration line and then the fluid-saturated portion of the test strip was immediately placed into a microcentrifuge on ice. Collected samples were stored at −20 Celsius before being shipped to a testing facility on dry ice.

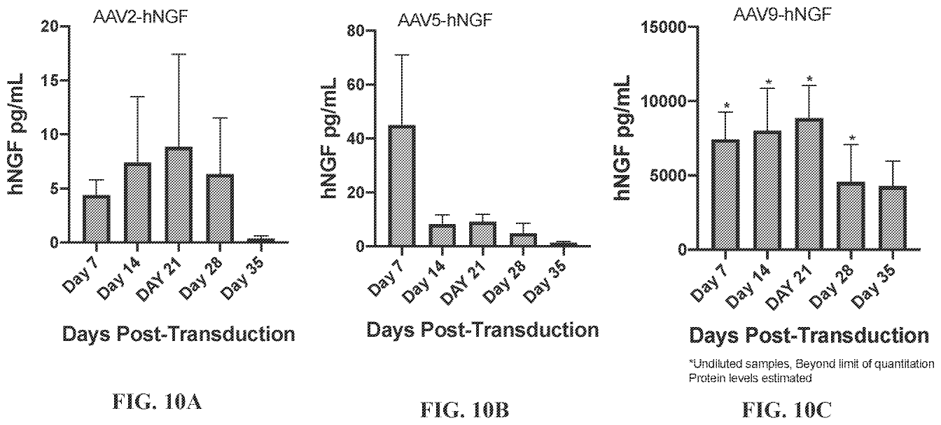

Protein was extracted from the Schirmer Tear Test strips of the left eye and a mesoscale discovery assay detecting human nerve growth factor protein (hNGFβ) was conducted on each sample. The minimum and maximum standard curve values of the MSD assay for human NGFβ were 0.104 to 427 pg/mL, respectively. Transduction of (comprising the expression cassette of SEQ ID NO: 25; depicted in FIG. 9) generated expression of hNFGβ in tears (FIGS. 10A-10C, respectively). The AAV9-hNGFβ transduction generated such high expression that the sample had to be diluted 16-128× for detection (Table 8). The average calculated tear film level of hNGFβ from AAV9-hNGFβ transduced pigs derived from the dilution study is 5,612.84 pg/mL. It should be noted that hNGFβ was not detected in non-transduced porcine tear film based upon hNGF MSD data collected from previous studies utilizing AAV vectors encoding different proteins. Therefore, any hNGFβ protein detected from extracts of the Schirmer strip tear samples was easily identified during the MSD assay and attributed to transduction of the lacrimal gland by AAV-hNGFβ constructs. The samples collected on Day 21 for AAV2-hNGFβ (FIG. 10A) includes an outlier (p<0.05 as determined by Grubb's test) which was omitted from the analysis. No significant decrease was observed between Days 7-35 for AAV9-hNGFβ as shown in FIG. 10C). Additionally, hNGF protein levels were compared between samples from animals administered AAV9-hNGFβ collected on Day 21 (no nasal spray), Day 28 (nasal spray administration), and Day 35 (no nasal spray). Administration of nasal spray increased tear hNGFβ protein concentration when administered the nasal spray compared to AAV administration alone (data not shown).

Together, this data demonstrates transduction of the porcine lacrimal gland with an adenoviral-associated vector encoding hNGFβ resulted in expression, secretion, and transport of hNGFβ to the tear film in detectable quantities. Expression was detectable within 7 days of lacrimal gland transduction with significant variability in expression levels dependent upon the AAV serotype utilized. AAV9 demonstrated the highest level of expression within 7 days and AAV2 showed the lowest level, albeit still within the range of the analytical assay utilized for detection of human nerve growth factor beta protein. AAV2 protein levels in tears is steadily increased out to 28 days, while AAV5 decreased after the first 7 days and appeared to have reached a steady-state at Days 14 through 28 with further reduction at day 35. AAV9 transduced animals had a significant level of hNGF detected in the tear film at Day 7 that remained constant out to Day 35.

TABLE 9
Serial dilution of AAV9-hNGFβ tear sample

| Dilution of | hNGFβ Concentration | |
|---|---|---|
| AAV9-hNGF Tears | (pg/mL) | Range of Detection |
| 1:2 | 8,729.83 | Above Upper Assay Limit |
| 1:4 | 10,001.39 | Above Upper Assay Limit |
| 1:8 | 7,613.18 | Above Upper Assay Limit |
| 1:16 | 6,439.27 | Within Range of Detection |
| 1:32 | 5,499.16 | Within Range of Detection |
| 1:64 | 4,763.83 | Within Range of Detection |
| 1:128 | 5,749.08 | Within Range of Detection |

### Example 4: Expression of EGFP Transgene in Porcine Lacrimal Gland Delivered by rAAV Via Intralacrimal Injection

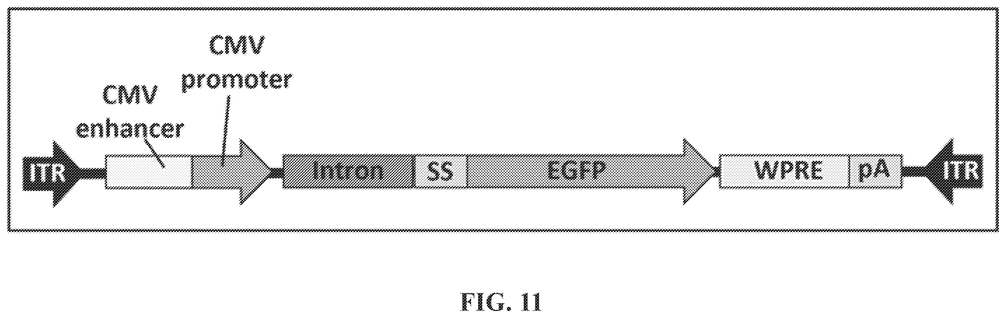

The study objective was to assess if the lacrimal gland is able to be leveraged as a method to modify or enrich the tear film with a protein of interest in pigs. Subsequently, in vivo study was performed to test if EGFP could be produced in the acinar cells of the lacrimal gland and then secreted into the tear film after delivery of an adenoviral vector consisting of a plasmid encoding eGFP. To get cDNA encoding EGFP into acinar cells, our approach was to inject the lacrimal gland with an adeno-associated virus (AAV) which contained cDNA encoding for secreted EGFP (secEGFP). To create each AAV of 2 different serotypes (2 and 9) for secEGFP, an AAV transfer plasmid was generated which contained between the inverted terminal repeats (ITRs) the essential elements for secEGFP expression. The DNA sequence between the ITRs was packaged into the AAV (FIG. 11) that was manufactured.

Eleven serotypes of AAV have been identified with the best characterized being AAV2. AAV pseudotyping is the mixing of a capsid and genome from different serotypes to improve transduction efficiency and these serotypes are denoted with a slash. For example, AAV2/5 indicates a virus containing serotype 2 packaged in the capsid from serotype 5. AAV5 and AAV9 have been reported to be capable of delivering a luciferase reporter gene to the lacrimal gland of mice (Rocha et al., Transduction, Tropism, and Biodistribution of AAV Vectors in the Lacrimal Gland, 2011; 52(13):9567-9572). Mouse lacrimal gland transduction with GFP as well as mouse nerve growth factor (mNGF) has been observed for pseudotypes AAV2/5 and AAV2/9 and reported recently on a pre-print server site (Gautier et al., AAV2/9-mediated gene transfer into murine lacrimal gland leads to a long-term targeted tear film modification; bioRxiv, 2022).

### Design Analysis and Methodology

Research grade AAVs for secreted EGFP (serotypes 2 and 9) were synthesized at Sirion and were provided at a stock concentration of 5×10¹². The AAVs were in vitro tested by CJ Solutions using HEK 293T cells and ELISA to ensure that the manufactured AAVs would transduce cells. At Texas A&M, eight domestic pigs received a one-time intralacrimal gland injection of EGFP with the right (OD; oculus dexter) gland receiving a low dose and the left (OS; oculus sinister) gland receiving a high dose. Six weeks after the first injection, a second injection with AAV2 and AAV9 high doses were performed. The study assessed EGFP expression at Day 35. Following tear EGFP level confirmation, the study was terminated 8 weeks after the second injection to assess the presence of EGFP in the lacrimal glands and assess any potential inflammation or gland abnormalities. (Tables 10 & 11). In the study, nasal spray dosing was administered between weeks 3 to 4 (Table 12).

TABLE 10
Study plan for in vivo study of AAV2-secEGFP and AAV9-secEGFP in domestic pigs.

| Title | | Fifteen week porcine study of AAV2-secEGFP and AAV9- |
|---|---|---|
| | | secEGFP following repeat intralacrimal gland injection |
| | | and OC-01 nasal spray administration. |
| GLP Status | | No |
| Objective(s) | | Fifteen week in vivo study to assess lacrimal gland |
| | | transduction following two intralacrimal gland injections |
| | | given 6 weeks apart. An initial of low-dose (1 × 10⁹ vg)- |
| | | or high-dose (1 × 10¹¹ vg) of AAV2-secEGFP or AAV9- |
| | | secEGFP was administered at Day 0 to transduce the |
| | | porcine lacrimal gland followed by a high dose (1 × 10¹¹ vg) |
| | | at week 6 (Day 47). The results are needed to: 1) confirm |
| | | expression and secretion of EGFP; 2) determine if EGFP is |
| | | detectable up to 8 weeks post-injection; and 3) assess any |
| | | AAV relatedsafety signals following repeat administration. |
| Study | Breed | Domestic pig |
| Animals | Species | Sus scrofa domesticus |
| | Age | 8-10 weeks |
| | Body Weight | ~18-23 kg |
| | Sex-(#M/#F) | 8 males |
| | Total | 8 for study (+naïve) |

| | |
|---|---|
| Test article(s) | First Injection- AAV2-secEGFP injected at a 1.0 × 10⁹ vg |
| | dose (OD) or 1.0 × 10¹¹ vg dose (OS) into the lacrimal gland - |
| | AAV9-secEGFP injected at a 1.0 × 10⁹ vg dose (OD) or |
| | 1.0 × 10¹¹ vg dose (OS) into the lacrimal gland |
| | Second injection: AAV2-secEGFP and AAV9-secEGFP at |
| | a 1 × 10¹¹ vg dose |
| Route of Administration | Intralacrimal gland injection for AAV and intranasal dose |
| | OC-01 |
| Study Groups and Dose | See Table Below |
| Levels | |
| Dosing Regimen | 1. First injection into each lacrimal gland; |
| | OD: single injection of 40 μL low dose |
| | OS: single injection of 40 μL high dose |
| | 2. Nasal spray given twice daily from day 21 to day 28 |
| | 3. Second injection into each lacrimal gland |
| | 4. |
| Randomization | No |
| Planned Sacrifice Timepoint | Day 103 |

TABLE 11
Injected dose and volume of AAV.

| | | | Dose | | | Number |
|---|---|---|---|---|---|---|
| | | | number | | | of |
| | | | and | Route | | Animals |
| | Dose Level | Injection | volume | (Right | Route | (gender) |
| Group | (vg) | Time | (μL) | Gland) | (Left Gland) | M |
| 1 | OD: low dose | Day 0 | OD: | Intralacrimal | Intralacrimal | 4 |
| | 1.0 × 10⁹ | | 40 μL | AAV2- | AAV2- | |
| | OS: high dose | | single | secEGFP | secEGFP | |
| | 1.0 × 10¹¹ | | injection | OD: 40 μL | OS: 40 μL | |
| | | | OS: | single injection | single injection | |
| | | | 40 μL | low dose | high dose | |
| | | | single | | | |
| | | | injection | | | |
| 2 | OD: low dose | Day 0 | OD: | Intralacrimal | Intralacrimal | 4 |
| | 1.0 × 10⁹ | | 40 μL | AAV9- | AAV9- | |
| | OS: high dose | | single | secEGFP | secEGFP | |
| | 1.0 × 10¹¹ | | injection | OD: 40 μL | OS: 40 μL | |
| | | | OS: | single injection | single injection | |
| | | | 40 μL | low dose | high dose | |
| | | | single | | | |
| | | | injection | | | |
| 1 | OD: high dose | Day 47 | OD: | Intralacrimal | Intralacrimal | 4 |
| | 1 × 10¹¹ | | 40 μL | AAV9- | AAV9- | |
| | OS: high dose | | single | secEGFP | secEGFP | |
| | 1.0 × 10¹¹ | | injection | OD: 40 μL | OS: 40 μL | |
| | | | OS: | single injection | single injection | |
| | | | 40 μL | | | |
| | | | single | | | |
| | | | injection | | | |
| 2 | OD: high dose | Day 47 | OD: | Intralacrimal | Intralacrimal | 4 |
| | 1 × 10¹¹ | | 40 μL | AAV2- | AAV2- | |
| | OS: high dose | | single | secEGFP | secEGFP | |
| | 1.0 × 10¹¹ | | injection | OD: 40 μL | OS: 40 μL | |
| | | | OS: | single injection | single injection | |
| | | | 40 μL | | | |
| | | | single | | | |
| | | | injection | | | |

vg = viral genomes

TABLE 12
OC-01 nasal spray dosing was from day 21 to day 28.

| | | | | Number of |
|---|---|---|---|---|
| | | | | Animals |
| | OC-01 | | | (gender) |
| Group | Dose Level | Dose | Dose number | Male |
| 1- Bilateral nares dose | 0.05 mL of | BID | Twice daily | 4 |
| | 30 mcg | | from day 21-28 | |
| 2- Bilateral nares dose | 0.05 mL of | BID | Twice daily | 4 |
| | 30 mcg | | from day 21-28 | |

mcg=micrograms

Following the second AAV-secEGFP injection, tears were collected from each eye via Schirmer strips on day 82. Tears were collected by placing a Schirmer's Tear Test strip in the lower conjunctival cul-de-sac and leaving in place for 2 minutes. Tear protein was extracted from the Schirmer's Tear Test strip and mesoscale discovery (MSD) analysis was conducted to detect the presence of EGFP protein in the tears.

Lacrimal gland was collected for ocular histopathology on Day 103 and samples were sent to Zyagen, Inc. (San Diego, CA) for EGFP immunohistochemistry (IHC).

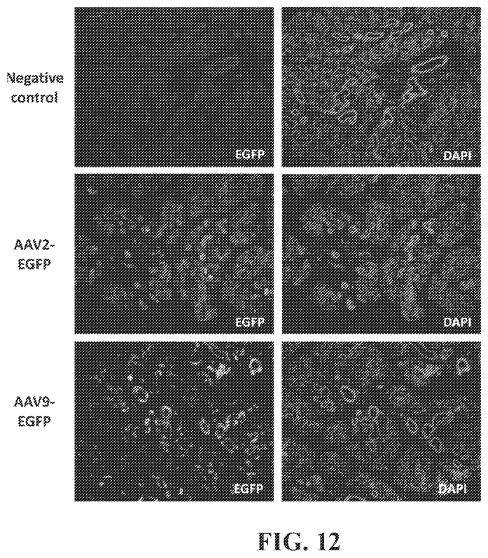

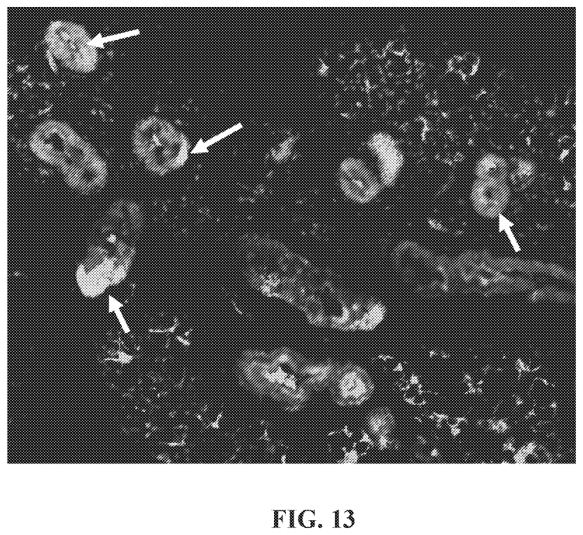

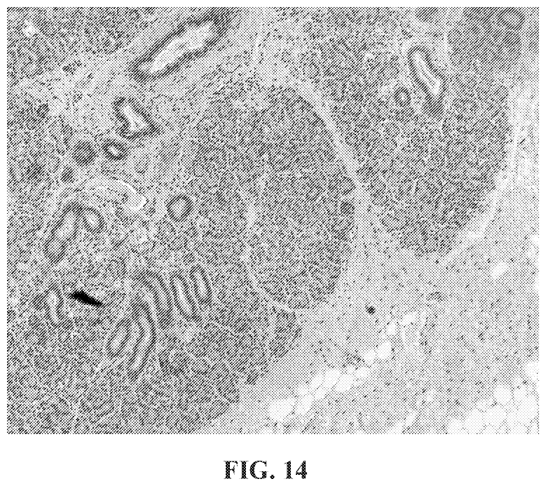

ELISA results showed that the AAV serotypes that were manufactured could transduce HEK 293T cells and produced secreted EGFP in vitro. EGFP expression in the tear samples was confirmed by MSD analysis 82 days after AAV transduction with eGFP some levels >400 pg/mL as well as by IHC (FIG. 12). IHC indicated that EGFP expression was within the acinar cells with greater acinar cell infectivity observed for AAV2 compared to AAV9. Additionally, transduction of ductile epithelial cells was observed for AAV9 injected lacrimal glands (FIG. 13). Hematoxylin and eosin staining of pig lacrimal gland after repeat AAV injections did not show any inflammatory infiltrate, atrophy or edema (FIG. 14).

Porcine lacrimal gland that is injected with either AAV2-secEGFP or AAV9-secEGFP expressed the EGFP transgene product in acinar cells as well as ductile epithelial cells. The EGFP that was expressed in the lacrimal gland was found to be secreted into the tear film. Additionally, no safety signals or inflammatory infiltrates were observed in any animals after repeat injections of AAV2 or AAV9 regardless if they initially received a low or high dose of AAV during the first injection. The results of this study demonstrate that the acinar cells of the lacrimal gland are a target for a gene therapy approach to modify and/or enrich the tear film.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating an ocular surface disorder, cornea disorder, or anterior chamber disorder in a subject, comprising:
(a) administering an effective amount of a primary therapeutic agent to the eye or a tissue in fluidic communication with the eye; and
(b) administering a treatment that increases tear production.

2. The method of claim 1, wherein the treatment that increases tear production comprises an effective amount of a nicotinic acetylcholine receptor (“nAChR”) agonist, or a pharmaceutically acceptable salt thereof.

3. The method of claim 2, wherein the nAChR agonist is
(a) a full agonist of nAChR subtypes alpha4beta2, alpha3beta4, alpha3alpha5beta4, and/or alpha4alpha6beta2;
(b) varenicline, or a pharmaceutically acceptable salt thereof; and/or
(c) (R)-5-((E)-2-pyrrolidin-3-ylvinyl)pyrimidine, or a pharmaceutically acceptable salt thereof.

4. (canceled)

5. (canceled)

6. The method of claim 1, wherein the treatment that increases tear production is administered via local nasal administration.

7. The method of claim 6, wherein the local nasal administration is via an intranasal spray.

8. The method of claim 1, wherein the primary therapeutic agent is administered via intralacrimal injection into one or both lacrimal glands or by topical administration.

9. The method of claim 1, wherein the treatment that increases tear production is administered subsequent to administration of the primary therapeutic agent.

10. The method of claim 9, wherein the treatment that increases tear production is administered beginning 4, 5, 6, 7, 8, 9 or 10 days subsequent to administration of the primary therapeutic agent.

11. The method of claim 1, wherein the treatment that increases tear production is an effective electrical stimulation of the anterior ethmoidal nerve.

12. The method of claim 1, wherein the tissue in fluidic communication with the eye is one or any combination of the lacrimal gland, the lacrimal duct, the cornea, cornea epithelium, limbal stem cells, conjunctiva, lacrimal puncta, and lacrimal canaliculi.

13. The method of claim 1, wherein a tissue in fluidic communication with the eye is a cornea and/or a lacrimal gland.

14. (canceled)

15. The method of claim 1, wherein the method results in an increased amount of the primary therapeutic agent delivered to the tear film of an eye of the subject in a predetermined time compared to administration of the primary therapeutic agent alone.

16. The method of claim 1, wherein the method results in an increased amount of the primary therapeutic agent delivered to the cornea of an eye of the subject in a predetermined time compared to administration of the primary therapeutic agent alone.

17. The method of claim 15, wherein the predetermined time is between about 5 minutes to about 1 hour.

18. (canceled)

19. The method of claim 1, wherein administering an effective amount of the primary therapeutic agent comprises administering an effective amount of a composition comprising a viral vector comprising a polynucleotide encoding a gene product, or functional variant thereof, to at least one lacrimal gland of the subject, wherein the primary therapeutic agent comprises the gene product, or functional variant thereof.

20. The method of claim 19, wherein
(a) the gene product is a therapeutic protein selected from Nerve Growth Factor (“NGF”) and Glial Derived Neurotrophic Factor (“GDNF”).

21. The method of claim 19, wherein
(a) the viral vector is an adeno-associated virus (“AAV”) vector;
(b) the viral vector is a vaccinia virus vector;
(c) the viral vector is an adenovirus vector; or
(d) the viral vector is a lentiviral vector.

22-26. (canceled)

27. The method of claim 20, wherein the viral vector is an AAV, and the AAV vector is an AAV of serotype 5 (“AAV5”), serotype 2 (“AAV2”), serotype 8 (“AAV8”), or serotype 9 (“AAV9”).

28. The method of claim 20, wherein the composition comprises
(a) about 1×10¹² genome copies per milliliter (“GC/mL”) of the AAV vector; or
(b) about 6.2×10¹² GC/mL of the AAV vector.

29. (canceled)

30. The method of claim 19, wherein
(a) about 50 μL to about 100 μL of the composition is administered;
(b) the method results in expression of the gene product in the tear film of an eye of the subject;
(c) the method results in expression of the gene product in the cornea of an eye of the subject; and/or
(d) the method results in an increased amount of the gene product delivered to the tear film in a predetermined time compared to administration of the viral vector alone.

31-33. (canceled)

34. The method of claim 30, wherein the method results in an increased amount of the gene product delivered to the tear film in a predetermined time compared to administration of the viral vector alone, and the predetermined time is between about 5 minutes to about 1 hour.

35. (canceled)

36. The method of claim 1, wherein the ocular surface disorder, cornea disorder, or anterior chamber disorder in the subject is neurotrophic keratitis, chemical burn of the ocular surface, corneal wound, corneal ulcer, persistent epithelial defect, dry eye disease, herpes simplex viral infection of the trigeminal nerve and/or the eye, varicella zoster virus infection of the trigeminal nerve and/or the eye, or diabetic complications of a corneal nerve.

37. A method of delivering a gene product to the ocular surface to at least one eye of a subject in need thereof, comprising:
(a) administering an effective amount of a composition comprising a viral vector comprising a polynucleotide encoding a gene product, or functional variant thereof, to at least one lacrimal gland of the subject; and
(b) administering via local nasal administration an effective amount of a nicotinic acetylcholine receptor (“nAChR”) agonist, or a pharmaceutically acceptable salt thereof.

38-64. (canceled)

65. A pharmaceutical composition for increasing tear production, comprising:
a viral vector comprising a polynucleotide encoding a gene product, or functional variant thereof, wherein the gene product is selected from Nerve Growth Factor (“NGF”) and Glial Derived Neurotrophic Factor (“GDNF”), and
a pharmaceutically acceptable carrier;
wherein the pharmaceutical composition is formulated for administration onto the ocular surface or for nasal administration.

66-70. (canceled)

71. A kit comprising:
(a) a first composition comprising a viral vector comprising a polynucleotide encoding a gene product, or functional variant thereof; and
(b) a second composition comprising a nicotinic acetylcholine receptor (“nAChR”) agonist; and
instructions for treating or delaying progression of an ocular disease, disorder, or condition in a subject in need thereof.
